SEQUENCE LISTING (10 sequences)

SEQ ID NO: 1 - DNA, 19 bp, Artificial Sequence
  Description of Artificial Sequence Synthetic primer
tgctggatgagctgaccct

SEQ ID NO: 2 - DNA, 18 bp, Artificial Sequence
  modified_base (15): inosine
  Description of Artificial Sequence Synthetic primer
cggctggtgaaccangct

SEQ ID NO: 3 - DNA, 26 bp, Artificial Sequence
  Description of Artificial Sequence Synthetic primer
gcaggactatgaggagaagacaaaga

SEQ ID NO: 4 - DNA, 24 bp, Artificial Sequence
  Description of Artificial Sequence Synthetic primer
gctcctggctctttatctgatcca

SEQ ID NO: 5 - DNA, 22 bp, Artificial Sequence
  Description of Artificial Sequence Synthetic primer
aggatcgcattgtggaggtgaa

SEQ ID NO: 6 - DNA, 24 bp, Artificial Sequence
  Description of Artificial Sequence Synthetic primer
cctgagatgggatcactctgcatt

SEQ ID NO: 7 - DNA, 24 bp, Artificial Sequence
  Description of Artificial Sequence Synthetic primer
aagctgggaggcaaaggctacaag

SEQ ID NO: 8 - DNA, 24 bp, Artificial Sequence
  Description of Artificial Sequence Synthetic primer
caccctgttgttgtcacggtcaaa

SEQ ID NO: 9 - DNA, 22 bp, Artificial Sequence
  Description of Artificial Sequence Synthetic primer
tggaccagagaactgaccaacg

SEQ ID NO: 10 - DNA, 22 bp, Artificial Sequence
  Description of Artificial Sequence Synthetic primer
ctcactctcgaacgtagaccct

(19) United States
(12) Patent Application Publication
(10) Pub. No.: US 2007/0134687 A1
(43) Pub. Date: Jun. 14, 2007

(54) Focused microarray and methods of diagnosing cancer

(75) Inventors: Elias Georges, Laval (CA); Claudia Boucher Boucher, Ile Perrot (CA); Anne-Marie Bonneau, Laval (CA)

Correspondence Address: WILMER CUTLER PICKERING HALE AND DORR LLP, 60 STATE STREET, BOSTON, MA, 02109, US

(73) Assignee: Aurelium BioPharma Inc., Montreal (CA)

(21) Appl. No.: 11/520,476

(22) Filed: Sep. 12, 2006

Related U.S. Application Data
(60) Provisional application No. 60/716,090, filed on Sep. 12, 2005.

(51) Int. Cl. C12Q 1/68; C12M 1/34

(52) U.S. Cl. 435/6; 435/287.2

(57) ABSTRACT

Disclosed are methods for diagnosing cancer in a cell sample by detecting an increase in the levels of expression of marker genes in the cell sample as compared to the levels of expression of the same marker genes in a normal, nonneoplastic cell of the same tissue type. Also disclosed is a focused microarray device for diagnosis of cancer in a cell sample.

This application claims the benefit of priority to U.S. Provisional Application No. 60/716,090, filed Sep. 12, 2005, the specification of which is incorporated by reference in its entirety.

### FIELD OF THE INVENTION

The present invention relates generally to the field of medicine. More specifically, the invention pertains to a device and methods for detecting the development of cancer in cell samples isolated from a subject.

### BACKGROUND OF THE INVENTION

Cancer is one of the deadliest illnesses in the United States. It accounts for nearly 600,000 deaths annually in the United States, and costs billions of dollars for those who suffer from the disease. This disease is in fact a diverse group of diseases, which can originate in almost any tissue of the body. In addition, cancers may be generated by multiple mechanisms including pathogenic infections, mutations, and environmental insults (see, e.g., Pratt et al. (2005) Hum Pathol. 36(8): 861-70). The variety of cancer types and mechanisms of tumorigenesis add to the difficulty associated with treating a tumor, increasing the risk posed by the cancer to the patient's life and wellbeing.

Most cancers, however, manifest abnormal growth and the ability to move from an original site of growth to other tissues in the body (hereinafter termed “metastasis”) as clinical features of disease. Therefore, these clinical manifestations are used to diagnose cancer because they are applicable to all cancers. In many instances, a cancer diagnosis is made based on identifying cancer cells by their gross pathology through histological and microscopic inspection of the cells. Although the gross pathology of the cells can provide accurate diagnoses of the cells, the techniques used for such analysis are hampered by the time necessary to process the tissues and the skill of the technician analyzing the samples. These methodologies can lead to unnecessary delay in treating a growing tumor, thereby increasing the likelihood that a benign tumor will acquire metastatic characteristics. It is thus necessary to diagnose potentially cancerous growths as soon as possible to avoid the development of a potentially life threatening illness.

One potential method of increasing the speed and accuracy of cancer diagnoses is the use of genes as markers for neoplastic potential. Recent advances in molecular biology have identified genes involved in cell cycle control, apoptosis, and metabolic regulation (see, e.g., Isoldi et al. (2005) Mini Rev. Med. Chem. 5(7): 685-95). Mutations in many of these genes have also been shown to increase the likelihood that a normal cell will progress to a malignant state (see, e.g., Soejima et al. (2005) Biochem. Cell Biol. 83(4): 429-37). For example, mutations in p 53, which is a well-known tumor suppressor gene, have been associated with aberrant cell growth leading to neoplastic potential (see Li et al. (2005) World J. Gastroenterol. 11 (19): 2998-3001). Many mutations can affect the levels of expression of certain genes in the neoplastic cells as compared to normal cells.

There remains a need to identify an accurate and rapid means for diagnosing cancer in patients. Treatment efficacy would be improved by more efficient diagnoses of tissue samples. Furthermore, rapid diagnoses of cancerous tissues would allow clinicians to treat potential tumors prior to the metastasis of the cancer to other tissues of the body. Finally, a test that did not rely upon a particular technicians skill at identifying abnormal histological characteristics would improve the reliability of cancer diagnoses. There is, therefore, a need for new methods of diagnoses for cancer that are accurate, fast, and relatively easy to interpret.

### SUMMARY OF THE INVENTION

By analyzing the levels of expression of certain genes in a potentially neoplastic cell, the cell can be classified as either a non-neoplastic cell or a neoplastic cell. The invention is based in part upon the discovery that differential expression of certain genes occurs when a cell progresses to a neoplastic state. These gene expression patterns are therefore diagnostic for the presence of cancer in a cell sample. This discovery has been exploited to provide an invention that allows for the use of nucleic acids (hereinafter “capture probes”) to determine the expression of a multiplicity of certain select marker genes in a cell sample in order to diagnose the presence of neoplastic cells in the cell sample.

In one aspect, the invention provides a method of diagnosing cancer in a subject using a focused microarray. The microarray has a plurality of nucleic acid capture probes, and each capture probe is complementary to a marker gene from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, ATP synthase δ, PDI/ER-60 precursor, ATP synthase β, prostasin, cathepsin β, FAS, HSCP60, topoisomerase IIα, PCNA, ezrin, PDI, cathepsin D, A-CRABP II, HSC70, rad 23 homolog β, ETF3 subunit 2β, proteosome B1 subunit proprotein, β-tubulin, s1c9a3r1, prosolin, HSP60, HER-2, L-plastin, estrogen receptor α, HSP27, thioredoxine peroxidase I, calumenin, 14-3-3 eta chain, Ki 67, MRP1, “similar to stratifin,” UCHL-1, mammaglobin 2, cellular RNA binding protein, and annexin I. In addition, the method entails the detection of a level of expression in a cell sample of the selected marker genes by contacting the nucleic acid capture probes with nucleic acids from the cell sample so as to allow for the hybridization of the nucleic acid capture probes with the nucleic acids from the cell sample. The level of expression of the selected marker genes in the cell sample is compared to the level of expression of the same marker genes in a normal cell sample of the same tissue type. The presence of a cancer cell is indicated if the level of expression of five or more of the selected marker genes in the cell sample is greater than the level of expression of the same marker genes in the normal cell sample of the same tissue type.

In certain embodiments, the microarray has a plurality of nucleic acid capture probes that are complementary to marker genes selected from the group consisting of cytokeratin 18, cytokeratin 7, keratin 19, α-enolase, s1c9a3r1, TPI, and HER-2. In other embodiments, the plurality of nucleic acid capture probes is at least six. In still other embodiments, the plurality of nucleic acid capture probes is at least seven. In further embodiments, the presence of a cancer cell is indicated if the level of expression of six or more of the selected marker genes in the cell sample is greater than the level of expression of the same marker genes in the normal cell sample of the same tissue type. In still further embodiments, the presence of a cancer cell is detected if the level of expression of seven or more of the selected marker genes in the cell sample is greater than the level of expression of the same marker genes in the normal cell sample of the same tissue type. In yet further embodiments, the plurality of marker genes selected is at least six, and an increased level of expression of at least five marker genes in the cell sample compared to the level of expression in the normal cell sample of the same tissue type indicates that the cell sample is neoplastic. In still more embodiments, the plurality of marker genes selected is at least seven, and an increased level of expression of at least five marker genes in the cell sample compared to the level of expression in the normal cell sample of the same tissue type indicates that the cell sample is neoplastic. In additional embodiments, the plurality of marker genes selected is at least eight, and an increased level of expression of at least six marker genes in the cell sample compared to the level of expression in the normal cell sample of the same tissue type indicates that the cell sample is neoplastic.

In certain embodiments, the cell sample is from breast tissue. In other embodiments, the plurality of marker genes is keratin 19, s1c9a3r1, FABP 4, and HER-2. In certain embodiments, the cell sample is from ovarian tissue and the plurality is at least three marker genes from the group consisting of cytokeratin 18, cytokeratin 7, α-enolase, and TPI. In other embodiments, the plurality of marker genes is cytokeratin 18, cytokeratin 7, α-enolase, and TPI.

In particular embodiments, the comparison of the level of expression of the selected marker genes further comprises using a class prediction algorithm to differentiate the level of expression of the selected marker genes in the cell sample from the level of expression of the same marker genes in the normal cell sample of the same tissue type. In more particular embodiments, one or more class prediction algorithms is from the group consisting of compound covariate predictor, diagonal linear discriminant analysis, nearest neighbor predictor, nearest centroid predictor, and support vector machine predictor.

In certain embodiments, the presence of a cancer cell is indicated if the level of expression in the cell sample of at least one of the selected marker genes is at least two times the level of expression of the same marker gene in the normal cell sample of the same tissue type.

In other embodiments, the presence of a cancer cell is indicated if the level of expression in the cell sample of at least one of the selected marker genes is at least three times the level of expression of the same marker gene in the normal cell sample of the same tissue type. In still other embodiments, the presence of a cancer cell is indicated if the level of expression in the cell sample of at least one of the selected marker genes is at least four times the level of expression of the same marker gene in the normal cell sample of the same tissue type. In yet other embodiments, the presence of a cancer cell is indicated if the level of expression in the cell sample of at least one of the selected marker genes is at least five times the level of expression of the same marker gene in the normal cell sample of the same tissue type. In still more embodiments, the presence of a cancer cell is indicated if the level of expression in the cell sample of at least one of the selected marker genes is at least six times the level of expression of the same marker gene in the normal cell sample of the same tissue type.

In particular embodiments, the step of comparing the level of expression of the selected marker gene(s) comprises using one or more class prediction algorithms selected from the group consisting of compound covariate predictor, diagonal linear discriminant analysis, nearest neighbor predictor, nearest centroid predictor, and support vector machine predictor. In more particular embodiments, the subject is a human. In still more particular embodiments, the cancer that is detected is from the group consisting of ovarian carcinoma, serous adenocarcinoma, clear cell adenocarcinoma, endometrioid carcinoma, mucinous adenocarcinoma, breast adenocarcinoma, and infiltrating ductal carcinoma.

In another aspect, the invention provides a method of diagnosing breast cancer in a subject. The method entails the step of selecting at least six marker genes from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, PDI/ER-60 precursor, ATP synthase β, prostasin, cathepsin β, FAS, HSCP60, topoisomerase IIα, PCNA, ezrin, PDI, cathepsin D, A-CRABP II, HSC70, β-tubulin, s1c9a3r1, prosolin, HSP60, HER-2, L-plastin, estrogen receptor α, HSP27, thioredoxine peroxidase I, calumenin, GAPDH, 14-3-3 eta chain, and annexin I. Once the marker genes are selected, a level of expression of the selected marker genes in a breast cell sample is detected by contacting probes capable of binding or hybridizing with the marker genes isolated from the cell sample. The level of expression of the selected marker genes in the breast cell sample is compare to a level of expression of the same marker genes detected in a normal breast cell sample. Finally, the presence of breast cancer is indicated if the level of expression of three or more marker genes in the breast cell sample is greater than the level of expression for in the normal breast cell sample.

In certain embodiments, at least six marker genes are selected, and an increased level of expression of a plurality of at least five marker genes in the breast cell sample compared to the level of expression in the normal breast cell sample indicates the presence of breast cancer. In still other embodiments, at least seven marker genes are selected, and an increased level of expression of at least six marker genes in the breast cell sample compared to the level of expression in the normal breast cell sample indicates the presence of breast cancer. In particular embodiments, the plurality of marker genes comprises keratin 19, s1c9a3r1, and HER-2. In certain embodiments, the level of expression of marker genes is detected by nucleic acid capture probes attached to a solid support.

In particular embodiments, the breast cancer is a breast adenocarcinoma or an infiltrating ductal carcinoma. In more particular embodiments, the subject is a human. In still more particular embodiments, the presence of cancer is indicated if the level of expression in the breast cell sample of at least one of the selected marker genes is at least two times the level of expression of the same marker gene in the normal breast cell sample.

In certain embodiments, the marker genes are from the group consisting of cathepsin D, ezrin, keratin 19, s1c9a3r1, A-CRABP II, HER-2, and estrogen receptor α. In particular embodiments, the presence of cancer is indicated if the level of expression in the breast cell sample of at least one of the selected marker genes is at least three times the level of expression of the same marker genes in the normal breast cell sample. In other embodiments, the average level of expression is determined in a breast cell sample and a normal breast cell sample for at least three marker genes from the group consisting of cathepsin D, ezrin, keratin 19, s1c9a3r1, A-CRABP II, HER-2, and estrogen receptor α. In still other embodiments, the level of expression of the marker genes in the breast cell sample is compared to the level of expression of the same marker genes in the normal breast cell sample using one or more class prediction algorithms from the group consisting of compound covariate predictor, diagonal linear discriminant analysis, nearest neighbor predictor, nearest centroid predictor, and support vector machine predictor.

In certain embodiments, the marker genes are keratin 19, s1c9a3r1, and HER-2. In particular embodiments, the presence of cancer is indicated if the level of expression in the breast cell sample of at least one of the plurality of selected marker genes is at least four times the level of expression of the same marker genes in the normal breast cell sample. In certain embodiments, the marker genes are from the group consisting of keratin 19, s1c9a3r1, HER-2, and FABP 4. In other embodiments, the presence of cancer is indicated if the level of expression of keratin 19, s1c9a3r1, and HER-2 is at least four times greater in the breast cell sample than the level of expression for the same marker genes in the normal breast cell sample and the level of expression of FABP 4 is decreased in the breast cell sample as compared to the normal breast cell sample.

In certain embodiments, the step of comparing the level of expression of the selected marker genes further comprises using a class prediction algorithm to differentiate the level of expression of the selected marker genes in the cell sample from the level of expression of the same marker genes in the normal cell sample of the same tissue type. In other embodiments, the step of comparing the level of expression of the selected marker genes further comprises differentiating the level of expression of the selected marker genes in the cell sample from the level of expression of the same marker genes in the normal cell sample of the same tissue type using one or more class prediction algorithms from the group consisting of compound covariate predictor, diagonal linear discriminant analysis, nearest neighbor predictor, nearest centroid predictor, and support vector machine predictor. In certain embodiments, the level of expression of marker genes is determined using RT-PCR, PCR, nucleic acid blotting, dot blotting, or microarray.

In another aspect, the invention provides a method of diagnosing ovarian cancer in a subject. The method comprises the first step of selecting at least six marker genes from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, PDI/ER-60 precursor, ATP synthase β, prostasin, cathepsin β, FAS, HSCP60, topoisomerase IIα, PCNA, ezrin, PDI, cathepsin D, A-CRABP II, and annexin I. The method further entails detecting a level of expression of the selected marker genes in an ovarian cell sample by contacting probes capable of binding or hybridizing with the marker genes isolated from the cell sample. Finally, the method includes the step of comparing the level of expression of the selected marker genes in the ovarian cell sample to a level of expression of the same marker genes detected in a normal ovarian cell sample. The presence of ovarian cancer is indicated if the level of expression of two or more marker genes in the ovarian cell sample is greater than the level of expression of the same marker genes in the normal ovarian cell sample.

In certain embodiments, the plurality of marker genes selected is at least six, and an increased level of expression of a plurality of at least four marker genes in the ovarian cell sample compared to the level of expression in the normal ovarian cell sample indicates the presence of ovarian cancer. In other embodiments, the plurality of marker genes selected is at least seven, and an increased level of expression of a plurality of at least five marker genes in the ovarian cell sample compared to the level of expression in the normal ovarian cell sample indicates the presence of ovarian cancer.

In certain embodiments, the marker genes consist of at least three marker genes from the group consisting of cytokeratin 18, cytokeratin 7, TPI, and α-enolase. In other embodiments, the marker genes are cytokeratin 18, cytokeratin 7, TPI, and α-enolase. In still other embodiments, the step of comparing the level of expression of marker genes in the ovarian cell sample to the level of expression of the marker genes in the normal ovarian cell sample comprises using one or more class prediction algorithms from the group consisting of compound covariate predictor, diagonal linear discriminant analysis, nearest neighbor predictor, nearest centroid predictor, and support vector machine predictor.

In particular embodiments, the level of expression of marker genes is detected by nucleic acid capture probes attached to a solid support. In more particular embodiments, the ovarian cancer cell is an ovarian carcinoma, a serous adenocarcinoma, a clear cell adenocarcinoma, an endometrioid carcinoma, or a mucinous adenocarcinoma. In still more particular embodiments, the subject is a human.

In certain embodiments, the presence of cancer is indicated if the level of expression in the ovarian cell sample of at least one of the selected marker genes is at least three times the level of expression of the same marker gene in the normal ovarian cell sample. In other embodiments, the marker genes are from the group consisting of HDGF, cytokeratin 18, α-enolase, GAPDH, GST-ρ, TPI, keratin 19, cytokeratin 7, pyrophosphatase inorganic, ATP synthase β, prostasin, cathepsin β, cathepsin D, and A-CRABP II. In particular embodiments, the presence of cancer is indicated if the level of expression in the ovarian cell sample of at least one of the selected marker genes is at least four times the level of expression of the same marker genes in the normal ovarian cell sample. In other embodiments, the presence of cancer is indicated if a level of expression in the ovarian cell sample of at least two marker genes is at least four times the level of expression for the same marker genes in the normal ovarian cell sample.

In certain embodiments, at least five of the marker genes are from the group consisting of HDGF, cytokeratin 18, α-enolase, GAPDH, GST-π, TPI, keratin 19, cytokeratin 7, and A-CRABP II. In other embodiments, the presence of cancer is indicated if the level of expression in the ovarian cell sample of at least one of the plurality of marker genes is at least five times the level of expression of the same marker genes in the normal ovarian cell sample. In still other embodiments, the presence of cancer is indicated if levels of expression are determined for all marker genes in the ovarian cell sample and the normal ovarian cell sample, and the levels of expression for all marker genes in the ovarian cell sample are at least five times the levels of expression for the same marker genes in the normal ovarian cell sample. In further embodiments, the step of comparing the levels of expression of marker genes in the ovarian cell sample to the levels of expression of the marker genes in the normal ovarian cell sample comprises using a class prediction algorithm.

In certain embodiments, the marker genes are from the group consisting of cytokeratin 18, α-enolase, TPI, and cytokeratin 7. In other embodiments, the presence of cancer is indicated if the level of expression in the ovarian cell sample of at least one of the plurality of selected marker genes is at least six times the level of expression of the same marker genes in the normal ovarian cell sample. In still other embodiments, the presence of cancer is indicated if levels of expression are determined for all marker genes in the ovarian cell sample and the normal ovarian cell sample, and the levels of expression for all marker genes in the ovarian cell sample are at least five times the levels of expression for the same marker genes in the normal ovarian cell sample. In further embodiments, the step of comparing the levels of expression of marker genes in the ovarian cell sample to the levels of expression of the marker genes in the normal ovarian cell sample comprises using a class prediction algorithm.

In particular embodiments, the step of comparing the level of expression of the selected marker genes further comprises using a class prediction algorithm to differentiate the level of expression of the selected marker genes in the ovarian cell sample from the level of expression of the same marker genes in the normal ovarian cell sample. In more particular embodiments, the step of comparing the level of expression of the selected marker genes further comprises differentiating the level of expression of the selected marker genes in the ovarian cell sample from the level of expression of the same marker genes in the normal ovarian cell sample using one or more class prediction algorithms from the group consisting of compound covariate predictor, diagonal linear discriminant analysis, nearest neighbor predictor, nearest centroid predictor, and support vector machine predictor. In certain embodiments, the level of expression of marker genes is determined using RT-PCR, PCR, nucleic acid blotting, dot blotting, or microarray.

In another aspect, the invention provides a focused microarray for diagnosing a neoplasm. The focused microarray comprises a first set of nucleic acid capture probes that comprise a plurality of nucleic acid capture probes, which are complementary to a marker gene from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, ATP synthase 6, PDI/ER-60 precursor, ATP synthase β, prostasin, cathepsin β, FAS, HSCP60, topoisomerase IIα, PCNA, ezrin, PDI, cathepsin D, A-CRABP II, HSC70, rad 23 homolog β, ETF3 subunit 2β, proteosome B1 subunit proprotein, β-tubulin, s1c9a3r1, prosolin, HSP60, HER-2, L-plastin, estrogen receptor α, HSP27, thioredoxine peroxidase I, calumenin, 14-3-3 eta chain, Ki 67, MRP1, “similar to stratifin,” UCHL-1, mammaglobin 2, cellular RNA binding protein, and annexin I. The focused microarray further contains a second set of nucleic acid capture probes that comprise a plurality of nucleic acid capture probes. Each capture probe is complementary to a marker gene encoding an endogenous housekeeping gene. Also, a solid support is provided to which the first and second set of nucleic acid capture probes are attached at predetermined positions.

In certain embodiments, the first set of capture probes is complementary to at least three of the marker genes from the group consisting of cytokeratin 7, cytokeratin 18, TPI, α-enolase, keratin 19, s1c9a3r1, and HER-2. In other embodiments, the first set of capture probes is complementary to at least four of the marker genes from the group consisting of cytokeratin 7, cytokeratin 18, TPI, α-enolase, keratin 19, s1c9a3r1, and HER-2. In still other embodiments, the first set of capture probes is complementary to at least five marker genes from the group consisting of cytokeratin 7, cytokeratin 18, TPI, α-enolase, keratin 19, s1c9a3r1 and HER-2. In yet other embodiments, the first set of capture probes is complementary to at least six of the marker genes from the group consisting of cytokeratin 7, cytokeratin 18, TPI, α-enolase, keratin 19, s1c9a3r1, and HER-2.

In certain embodiments, at least one capture probe of the second set is complementary to a marker gene from the group consisting of EF-2 and EIF-4B. In other embodiments, the second set of capture probes consists of the marker genes EF-2 and EIF-4B. In still other embodiments, the first set of capture probes is complementary to marker genes from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, PDI/ER-60 precursor, ATP synthase β, prostasin, cathepsin β, FAS, HSCP60, topoisomerase IIα, PCNA, ezrin, PDI, cathepsin D, A-CRABP II, HSC70, β-tubulin, s1c9a3r1, prosolin, HSP60, HER-2, L-plastin estrogen receptor α, HSP27, thioredoxine peroxidase I, calumenin, GAPDH, 14-3-3 eta chain, and annexin I. In yet other embodiments, the first set of capture probes is complementary to marker genes from the group consisting of keratin 19, s1c9a3r1, and HER-2.

In certain embodiments, the first set of capture probes is complementary to marker genes from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, PDI/ER-60 precursor, ATP synthase β, prostasin, cathepsin ρ, FAS, HSCP60, topoisomerase IIα, PCNA, ezrin, PDI, cathepsin D, A-CRABP II, and annexin I. In other embodiments, the first set of capture probes is complementary to marker genes from the group consisting of cytokeratin 18, cytokeratin 7, TPI, and cytokeratin 7.

In yet another aspect, the invention provides a kit for diagnosing cancer in a subject. The kit provides a first set of probes for the detection of one or more marker genes from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, ATP synthase δ, PDI/ER-60 precursor, ATP synthase β, prostasin, cathepsin β, FAS, HSCP60, topoisomerase IIα, PCNA, ezrin, PDI, cathepsin D, A-CRABP II, HSC70, rad 23 homolog β, ETF3 subunit 2β, proteosome B1 subunit proprotein, β-tubulin, s1c9a3r1, prosolin, HSP60, HER-2, L-plastin, estrogen receptor α, HSP27, thioredoxine peroxidase I, calumenin, 14-3-3 eta chain, Ki 67, MRP1, “similar to stratifin,” UCHL-1, mammaglobin 2, cellular RNA binding protein, and annexin I. The kit also provides a second set of probes for the detection of one or more marker genes from endogenous housekeeping genes. Furthermore, the kit contains a detection means for identifying a probe hybridizing to a target marker gene.

In certain embodiments, the first set of probes is a plurality of nucleic acids complementary to mRNA encoding the selected marker genes. In other embodiments, the nucleic acid is from the group consisting of single-stranded RNA, double-stranded RNA, double-stranded DNA, single-stranded DNA, and RNA-DNA hybrids.

In certain embodiments, the second set of probes is a plurality of nucleic acids complementary to an mRNA encoding marker genes that do not vary statistically significantly in level of expression between cancer cell samples and normal cell samples. In other embodiments, the second set of probes is a plurality of nucleic acids complementary to an mRNA encoding EF-2 and EIF-4B. In still other embodiments, the nucleic acid is selected from the group consisting of single-stranded RNA, double-stranded RNA, double-stranded DNA, single-stranded DNA, RNA-DNA hybrids, and siRNA.

In certain embodiments, the detection means is selected from the group consisting of fluorophores, chemical dyes, radiolabels, chemiluminescent compounds, colorimetric enzymatic reactions, chemiluminescent enzymatic reactions, magnetic compounds, and paramagnetic compounds. In particular embodiments, the first set and second set of probes are attached to a solid support at predetermined positions. In more particular embodiments, the cancer being detected is from the group consisting of breast adenocarcinoma, infiltrating ductal carcinoma, ovarian carcinoma, serous adenocarcinoma, clear cell adenocarcinoma, endometrioid carcinoma, and mucinous adenocarcinoma. In certain embodiments, a training set is provided that comprises a pamphlet supplying information on the levels of expression of the marker genes in a normal cell sample and a neoplastic cell sample detected by the first set of probes.

In another aspect, the invention provides a method of diagnosing breast cancer in a subject. The method comprises selecting the marker genes cathepsin D, ezrin, keratin 19, s1c9a3r1, A-CRAB II, HER-2 and estrogen receptor α. Next, a level of expression for the marker genes is detected in a breast cell sample by hybridizing probes with the marker genes isolated from the cell sample. The method also entails detecting a level of expression for the marker genes in a normal breast cell sample by hybridizing probes with the marker genes isolated from the normal breast cell sample. Finally, the level of expression for the marker genes in a breast cell sample is compared to the level of expression of the same marker genes in the normal breast cell sample. The presence of breast cancer is indicated if the level of expression of the marker genes in the breast cell sample is greater than the level of expression of the same marker genes in the breast normal cell sample.

### BRIEF DESCRIPTION OF THE FIGURES

The foregoing and other objects of the present invention, the various features thereof, as well as the invention itself may be more fully understood from the following description, when read together with the accompanying drawings in which:

FIG. 1A is a graphic representation showing the results of a microarray analysis comparing the levels of expression of several marker genes in normal breast tissue in a Caucasian population, an Asian population, and an African descent population.

FIG. 1B is a graphic representation showing the results of a microarray analysis comparing the levels of expression of several marker genes in normal breast tissue in a Caucasian population, an Asian population, and an African descent population.

FIG. 1C is a graphic representation showing the results of a microarray analysis comparing the levels of expression of several marker genes in normal breast tissue in a Caucasian population, an Asian population, and an African descent population.

FIG. 2A is a graphic representation showing the results of a microarray analysis comparing the levels of expression of several marker genes in normal ovarian tissue in a Caucasian population, an Asian population, and an African descent population.

FIG. 2B is a graphic representation showing the results of a microarray analysis comparing the levels of expression of several marker genes in normal ovarian tissue in a Caucasian population, an Asian population, and an African descent population.

FIG. 2C is a graphic representation showing the results of a microarray analysis comparing the levels of expression of several marker genes in normal ovarian tissue in a Caucasian population, an Asian population, and an African descent population.

FIG. 3A is a photographic representation of a microarray experiment showing spot average on a microarray during experiments on normal breast tissue.

FIG. 3B is a graphic representation of a microarray experiment showing spot coverage on a microarray during experiments on normal breast tissue.

FIG. 3C is a graphic representation of a microarray experiment showing spot coverage on a microarray during experiments on normal breast tissue.

FIG. 4A is a graphic representation of a microarray experiment showing spot coverage on a microarray during experiments on normal ovarian tissue.

FIG. 4B is a graphic representation of a microarray experiment showing spot coverage on a microarray during experiments on normal ovarian tissue.

FIG. 4C is a graphic representation of a microarray experiment showing spot coverage on a microarray during experiments on normal ovarian tissue.

FIG. 5A is a graphic representation showing the results of a microarray analysis comparing the levels of expression of several marker genes in an RNA reference pool to an ovarian cancer patient RNA pool.

FIG. 5B is a graphic representation showing the results of a microarray analysis comparing the levels of expression of several marker genes in an RNA reference pool to an ovarian cancer patient RNA pool.

FIG. 5C is a graphic representation showing the results of a microarray analysis comparing the levels of expression of several marker genes in an RNA reference pool to an ovarian cancer patient RNA pool.

FIG. 6 is a graphic representation showing the results of microarray analyses of the average levels of expression of TPI, cytokeratin 7, cytokeratin 18, and α-enolase in normal tissues and tumor samples.

FIG. 7 is a graphic representation showing the results of microarray analyses of the average levels of expression of HDGF, GAPDH, keratin 19, and A-CRABP II in normal tissues and tumor samples.

FIG. 8 is a graphic representation showing the results of microarray analyses of the average levels of expression of TPI, cytokeratin 7, cytokeratin 18, α-enolase, HDGF, GAPDH, keratin 19, and A-CRABP II in normal tissues and tumor samples.

FIG. 9 is a graphic representation showing the accuracy of the classification of cells as either normal ovarian cells or neoplastic ovarian cells using marker gene expression levels in combination with classification algorithms.

FIG. 10A is a graphic representation showing the results of microarray analyses of the average levels of expression of α-enolase in normal tissues and tumor samples.

FIG. 10B is a graphic representation showing the results of microarray analyses of the average levels of expression of TPI in normal tissues and tumor samples.

FIG. 11 is a graphic representation showing the results of microarray analyses of the average levels of expression of cytokeratin 18 in normal tissues and tumor samples.

FIG. 12A is a graphic representation showing the results of microarray analyses of the average levels of expression of HDGF in normal tissues and tumor samples.

FIG. 12B is a graphic representation showing the results of microarray analyses of the average levels of expression of keratin 19 in normal tissues and tumor samples.

FIG. 13 is a graphic representation showing the results of microarray analyses of the average levels of expression of GAPDH in normal tissues and tumor samples.

FIGS. 14A and 14B are graphic representations showing the results of microarray analyses of the average levels of expression of cathepsin D in normal tissues and tumor samples.

FIG. 15A is a graphic representation showing the results of microarray analyses of the average levels of expression of cathepsin D, ezrin, keratin 19, s1c9a3r1, A-CRABP II, HER-2, estrogen receptor α, and FABP4 in normal tissues and tumor samples.

FIG. 15B is a graphic representation showing the results of microarray analyses of the average levels of expression of cathepsin D, ezrin, keratin 19, s1c9a3r1, A-CRABP II, estrogen receptor α, and FABP4 in normal tissues and tumor samples.

FIG. 16 is a graphic representation showing the accuracy of the classification of cells as either normal breast cells or neoplastic breast cells using marker gene expression levels in combination with classification algorithms.

FIG. 17 is a diagrammatic representation showing the microarray configuration that was used to test the ovarian tissue samples.

FIG. 18 is a diagrammatic representation showing the microarray configuration that was used to test the breast tissue samples.

FIG. 19 is a graphic representation showing the comparison of results of an RT-PCR analysis and a focused microarray analysis with each analysis comparing the levels of expression of keratin 19 in breast tumor samples and normal breast samples to an RNA reference pool (BRPool) to obtain a ratio for each sample.

FIG. 20 is a graphic representation showing the comparison of results of an RT-PCR analysis and a focused microarray analysis with each analysis comparing the levels of expression of ezrin in breast tumor samples and normal breast samples to an RNA reference pool (BRPool) to obtain a ratio for each sample.

FIG. 21 is a graphic representation showing the comparison of results of an RT-PCR analysis and a focused microarray analysis with each analysis comparing the levels of expression of s1c9a3r1 in breast tumor samples and normal breast samples to an RNA reference pool (BRPool) to obtain a ratio for each sample.

FIG. 22 is a graphic representation showing the comparison of results of an RT-PCR analysis and a focused microarray analysis with each analysis comparing the levels of expression of cathepsin D in breast tumor samples and normal breast samples to an RNA reference pool (BRPool) to obtain a ratio for each sample.

FIG. 23 is a graphic representation showing the comparison of results of an RT-PCR analysis and a focused microarray analysis with each analysis comparing the levels of expression of ACRAB-PII in breast tumor samples and normal breast samples to an RNA reference pool (BRPool) to obtain a ratio for each sample.

FIG. 24 is a graphic representation showing the comparison of results of an RT-PCR analysis and a focused microarray analysis with each analysis comparing the levels of expression of the keratin 19, ezrin, s1c9a3r1, cathepsin D, and ACRAB-PII in breast tumor samples to an RNA reference pool (BRPool) to obtain a ratio for each sample.

FIG. 25 is a graphic representation showing the comparison of results of an RT-PCR analysis and a focused microarray analysis with each analysis comparing the levels of expression of the keratin 19, ezrin, s1c9a3r1, cathepsin D, and ACRAB-PII in breast normal samples to an RNA reference pool (BRPool) to obtain a ratio for each sample.

FIG. 26 is a graphic representation showing the comparison of results of an RT-PCR analysis and a focused microarray analysis with each analysis comparing the levels of expression of the keratin 19, ezrin, s1c9a3r1, cathepsin D, and ACRAB-PII in breast tumor samples and breast normal samples to an RNA reference pool (BRPool) to obtain a ratio for each sample.

FIG. 27 is a graphic representation showing the comparison of results of an RT-PCR analysis and a focused microarray analysis with each analysis comparing the levels of expression of the keratin 19, ezrin, s1c9a3r1, cathepsin D, and ACRAB-PII in breast tumor samples to an RNA reference pool (BRPool) to obtain a ratio for each sample.

FIG. 28 is a graphic representation showing the results of an efficiency analysis of PCR primers that shows the efficiency of amplifications of primers directed to keratin 19, ezrin, s1c9a3r1, cathepsin D, ACRAB-PII and Arabidopsis control primers.

FIG. 29 is a graphic representation showing the results of Quantitative RT-PCR analyses of individual patients that shows the levels of expression of keratin 19 in normal breast samples and tumor breast samples as compared to the BRPool of normal tissue samples to obtain a ratio for each sample.

FIG. 30 is a graphic representation showing the results of Quantitative RT-PCR analyses of individual patients that shows the levels of expression of ezrin in normal breast samples and tumor breast samples as compared to the BRPool of normal tissue samples to obtain a ratio for each sample.

FIG. 31 is a graphic representation showing the results of Quantitative RT-PCR analyses of individual patients that shows the levels of expression of cathepsin D in normal breast samples and tumor breast samples as compared to the BRPool of normal tissue samples to obtain a ratio for each sample.

FIG. 32 is a graphic representation showing the results of Quantitative RT-PCR analyses of individual patients that shows the levels of expression of s1c9a3r1 in normal breast samples and tumor breast samples as compared to the BRPool of normal tissue samples to obtain a ratio for each sample.

FIG. 33 is a graphic representation showing the results of Quantitative RT-PCR analyses of individual patients that shows the levels of expression of A CRAB-PII in normal breast samples and tumor breast samples as compared to the BRPool of normal tissue samples to obtain a ratio for each sample.

FIG. 34 is a graphic representation showing the comparison of results of an RT-PCR analysis and a focused microarray analysis with each analysis comparing the levels of expression of keratin 19 in breast tumor samples and normal breast samples to an RNA reference pool (BRPool) to obtain a ratio for each sample.

FIG. 35 is a graphic representation showing the comparison of results of an RT-PCR analysis and a focused microarray analysis with each analysis comparing the levels of expression of ezrin in breast tumor samples and normal breast samples to an RNA reference pool (BRPool) to obtain a ratio for each sample.

FIG. 36 is a graphic representation showing the comparison of results of an RT-PCR analysis and a focused microarray analysis with each analysis comparing the levels of expression of s1c9a3r1 in breast tumor samples and normal breast samples to an RNA reference pool (BRPool) to obtain a ratio for each sample.

FIG. 37 is a graphic representation showing the comparison of results of an RT-PCR analysis and a focused microarray analysis with each analysis comparing the levels of expression of cathepsin D in breast tumor samples and normal breast samples to an RNA reference pool (BRPool) to obtain a ratio for each sample.

FIG. 38 is a graphic representation showing the comparison of results of an RT-PCR analysis and a focused microarray analysis with each analysis comparing the levels of expression of ACRAB-PII in breast tumor samples and normal breast samples to an RNA reference pool (BRPool) to obtain a ratio for each sample.

### DETAILED DESCRIPTION OF THE INVENTION

The patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

1.1. General

An embodiment of the present invention in part provides methods and a device for diagnosing, detecting, or screening a cell sample for tumorigenic potential and neoplastic characteristics such as aberrant growth and metastatic potential. The invention also allows for the improved clinical management of tumors by providing a device that detects the expression level of genes identified as markers for cancer. Furthermore, embodiments of the invention provide a focused microarray that allows for rapid identification of cancer in a cell sample.

Accordingly, one aspect of the invention provides a focused microarray for diagnosis of cancer in a cell. The microarray has a plurality of capture probes that bind marker genes isolated from the cell sample. The nucleic acid capture probes are attached to a solid support at predetermined positions.

As used herein, the term “tumorigenic potential” means capable of giving rise to either benign or malignant tumors. Tumorigenic potential may occur through genetic mechanisms such as mutation or through infection with vectors such as viruses and bacteria.

As used herein, the term “cancer” refers to a disease condition in which a tissue or cells exhibit aberrant, uncontrolled growth. A cancer can be a single cell or, alternatively, a tumor composed of hyperplastic cells. In addition, cancers can be malignant, spreading from an original tumor to other tissues in the body. Cancers can also be localized to one tissue of the body.

As used herein, a “cancer cell” is a cell that shows aberrant cell growth, such as increased, uncontrolled cell growth. A cancer cell can be a hyperplastic cell, a cell from a cell line that shows a lack of contact inhibition when grown in vitro, a tumor cell when grown in vivo, or a cancer cell that is capable of metastasis in vivo. Non-limiting examples of cancer cells include melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, and thymoma, and lymphoma cells, melanoma cells, sarcoma cells, leukemia cells, retinoblastoma cells, hepatoma cells, myeloma cells, glioma cells, mesothelioma cells, and carcinoma cells.

The term “focused microarray” as used herein refers to a device that includes a solid support with capture probe(s) affixed to the surface of the solid support. The capture probes are directed to the diagnosis of a specific condition, e.g., cancer. Typically, the support consists of silicon, glass, nylon or metal alloy. Solid supports used for microarray production can be obtained commercially from, for example, Genetix Inc. (Boston, Mass.). Moreover, the support can be derivatized with a compound to improve nucleic acid association. Exemplary compounds that can be used to derivatize the support include aldehydes, poly-lysine, epoxy, silane containing compounds and amines. Derivatized slides can also be obtained commercially from Telechem International (Sunnyvale, Calif.).

The term “marker genes” as used herein means any group of nucleic acid sequences, whether chromosomal or extrachromosomal, that is utilized by a cancer cell to produce a “gene product,” which can or cannot produce a phenotype in the cancer cell or the organism. As used herein, “gene product” means any biomolecule that is produced from a nucleotide sequence or could be produced from a nucleotide sequence. Gene products include, but are not limited to, pre-messenger RNA, messenger RNA, transfer RNA, heteronuclear RNA (“HnRNA”), ribosomal RNA, single-stranded DNA, double-stranded RNA, peptides and proteins. Extrachromosomal sources of nucleic acid sequences can include double-strand DNA viral genomes, single-stranded DNA viral genomes, double-stranded RNA viral genomes, single-stranded RNA viral genomes, bacterial DNA, mitochondrial genomic DNA, cDNA or any other foreign source of nucleic acid that is capable of generating a gene product.

As used herein, the term “normal cell sample” refers to a cell or group of cells that is exhibiting common characteristics for the particular cell type from which the cell or group of cells was isolated. A normal cell sample does not exhibit tumorigenic potential, metastatic potential, or aberrant growth in vivo or in vitro. A normal cell sample can be isolated from tissues in a subject that is not suffering from cancer. It is not necessary to isolate a normal cell sample each time a cell sample is tested for cancer as long as the nucleic acids isolated from the normal cell sample allow for probing against the focused microarray during the testing procedure.

Alternatively, data related to the levels of expression of the selected marker genes in normal tissues and neoplasms can be supplied in a kit or individually in the form of a pamphlet, document, floppy disk, or computer CD. The data can represent patient pools developed for a particular population (e.g., Caucasian, Asian, etc.) and is tailored to a particular cancer type. Such data can be distributed to clinicians desiring to test patients for the presence of a neoplasm. A clinician would obtain the levels of expression for a marker gene or set of marker genes in a particular patient. The clinician would then compare the expression information obtained from the patient to the levels of expression for the same marker gene or set of marker genes that had been determined previously for both normal and cancer patient pools. A finding that the level of expression for the marker gene or the set of marker genes was similar to the normal patient pool data would indicate that the cell sample obtained from the patient is not neoplastic. A finding that the level of expression for the marker genes or the set of marker genes was similar to the cancer patient pool data would indicate that the cell sample obtained from the patient is neoplastic.

For purposes of the invention, the term “capture probe” is intended to mean any agent capable of binding a gene product in a complex cell sample. Capture probes can be disposed on the derivatized solid support utilizing methods practiced by those of ordinary skill in the art through a process called “printing” (see, e.g., Schena et. al., (1995) Science, 270(5235): 467-470). The term “printing”, as used herein, refers to the placement of spots onto the solid support in such close proximity as to allow a maximum number of spots to be disposed onto a solid support. The printing process can be carried out by, e.g., a robotic printer. The VersArray CHIP Writer Prosystem (BioRad Laboratories) using Stealth Micro Spotting Pins (Telechem International, Inc, Sunnyvale, Calif.) is a non-limiting example of a chip-printing device that can be used to produce the focused microarray for this aspect. In certain embodiments, capture probes are nucleic acids (herein termed “nucleic acid capture probes”) that are attached to a solid support at predetermined positions.

In the case of nucleic acid capture probes, nucleic acid sequences that are selected for attachment to the focused microarray may correspond to regions of low homology between genes, thereby limiting cross-hybridization to other sequences. Typically, this means that the sequences show a base-to-base identity of less than or equal to 30% with other known sequences within the organism being studied. Sequence identity determinations can be performed using the BLAST research program located at the NIH website (Altschul et al. (1990) J. Mol. Biol. 215: 403-410). Alternatively, the Needleman-Wunsch global alignment algorithm can be used to determine base homology between sequences (see Cheung et al., (2004) FEMS Immunol. Med. Micorbiol. 40(1): 1-9.). In addition, the Smith-Waterman local alignment can be used to determine a 30% or less homology between sequences (see Goddard et al., (2003) J. Vector Ecol. 28:184-9).

In another aspect, the invention provides methods for diagnosing cancer in a cell sample. The methods can be practiced using a microarray composed of capture probes affixed to a derivatized solid support such as, but not limited to, glass, nylon, metal alloy, or silicon. Non-limiting examples of derivatizing substances include aldehydes, gelatin-based substrates, epoxies, poly-lysine, amines and silanes. Techniques for applying these substances to solid surfaces are well known in the art. In useful embodiments, the solid support can be comprised of nylon. Such slides are particularly useful when utilizing synthetic oligonucleotides. For example, nylon supports have been used to produce short oligonucleotides directly to the support (see, e.g., Liou et. al. (2004) BMC Urol. 4(1): 9).

In certain embodiments, the levels of expression of the marker genes in the cell sample are compared to the levels of expression of the marker genes in a normal cell sample of the same tissue type as the cell sample. If the expression of at least one marker gene in the cell sample is greater than the expression of the marker gene or genes in the normal cell sample, then cancer is indicated. In some embodiments, the cell sample is tumorigenic if the level of expression of at least two or more of the plurality of marker genes in the cell sample is greater than the level of expression of the same marker gene(s) in the normal cell sample of the same tissue type.

The device can be incubated with labeled probes that correspond to any non-homologous sequences of the marker genes. Expression levels for the marker genes can be determined using techniques known in the art, such as, but not limited to, quantitative RT-PCR, microarrays, and RNA blotting (see, e.g., Rehman et al. (2004) Hum. Pathol. 35(11):1385-91; Yang et al. (2004) Mol. Biol. Rep. 31(4):241-8). Such examples are not intended to limit the potential means for determining the expression of a gene marker in a cell sample.

Non-homologous sequences pertaining to sequences identified in marker genes are utilized when using nucleic acid capture probes. Homology is determined by having a threshold homology of less than or equal to 30% for sequences utilized as probes. Homologies can be determined by the BLAST sequence alignment program located at the online site (www.ncbi.nlm.nih.gov/BLAST; Altschul et al. (1990) J. Mol. Biol. 215: 403-410), the Needleman-Wunsch global alignment algorithm, or the Smith-Waterman local alignment. The device can be incubated with unlabeled probes and indirect methods of detection can be used to identify the expression level of marker genes in a cell sample. Protein expression levels are determined by methods that specifically recognize a particular sequence of amino acids in the protein.

Cell samples can be isolated from human tumor tissues using means that are known in the art (see, e.g., Vara et al. (2005) Biomaterials 26(18):3987-93; Iyer et al. (1998) J. Biol. Chem. 273(5):2692-7). For example, the cell sample can be isolated from a human patient with breast cancer, or ovarian cancer, or lung cancer. Alternatively, cell samples can be obtained commercially from cell line sources as well (e.g., American Type Culture Collections, Mannassas, Va.).

As used herein, “breast cell sample” is intended to mean a cell that is isolated from breast tissue. Likewise, the term “ovarian cell sample” refers to a cell whose origins are from ovarian tissue. Breast cell samples can be isolated from several non-limiting types of breast tissue including glandular, ductal, stromal, fibrous and lymphatic tissue. In addition, the cell sample can be a metastatic cell isolated from bone, lymphatic tissue, blood, brain, lung, muscle, and skin. Breast or ovarian cell samples can be isolated from a mammal such as a human, mouse, rat, horse, pig, guinea pig, or chinchilla. The methods of the invention can be used to detect different types of neoplastic cells from breast tissue. Exemplary non-limiting breast cancer cells include lobular neoplasia, ductal carcinoma in situ, infiltrating lobular carcinoma, infiltrating ductal carcinoma, tubular carcinoma, mucinous carcinoma, medullary carcinoma, phylloides tumor, inflammatory breast cancer, Paget's disease of the nipple, ductal carcinoma, and breast adenocarcinoma. Breast cancer cell lines are also available from common sources, such as the ATCC cell biology collections (American Type Culture Collections, Mannassas, Va.).

The cell sample can be isolated from several non-limiting types of ovarian tissue including glandular, epithelial, lymphatic, sex cord, germ tissues, and stromal tissue. In addition, the cancer cell can be a metastatic cell isolated from bone, lymphatic tissue, blood, brain, breast, muscle, and skin. Ovarian cell samples can be isolated from a mammal such as a human, mouse, rat, horse, pig, guinea pig, or chinchilla. The methods of the invention can be used to detect different types of neoplastic cells from ovarian tissue. Exemplary non-limiting ovarian cancers detected by the invention include ovarian adenocarcinoma, epithelial adenocarcinoma, sex cord-stromal carcinoma, endometrioid tumors, mucinous carcinoma, germ cell tumors, and clear cell tumors. Alternatively, ovarian cancer cell lines can be used and are available from common sources such as the ATCC cell biology collections.

The present invention allows for the detection of cancer in tissues that are of mixed cellular populations such as a mixture of cancer cells and normal cells. In such cases, cancer cells can represent as little as 40% of the tissue isolated for the present invention to determine that the cell sample is tumorigenic. In certain embodiments, the cell sample can be composed of 50% cancer cells for the present invention to detect tumorigenic potential. Cell samples composed of greater than 50% tumorigenic cells can also be used in the present invention. It should be noted that cell samples can be isolated from tissues that are less than 40% tumorigenic cells as long as the cell sample contains a portion of cells that are at least 40% tumorigenic.

In useful embodiments, housekeeping genes are used to normalize a signal on the focused microarray. As used herein, the term “housekeeping genes” refers to any gene that has relatively stable or steady expression during the life of a cell. In addition, housekeeping genes can be marker genes that show little difference in expression between cancer cells and normal cells in a particular tissue type. Examples of housekeeping genes are well known in the art, such as isocitrate lyase, acyltransferase, creatine kinase, TATA-binding protein, hypoxanthine phosphoribosyl transferase 1, and guanine nucleotide binding protein, beta polypeptide 2-like 1 (see, e.g., Zhang et al. (2005) BMC Mol. Biol. 6:4). The housekeeping genes are used to identify the proper signal level by which to compare the cell sample signals between microarray experiments.

Another aspect of the invention provides a method of diagnosing cancer in a cell sample. In this method, expression of a marker gene in the potential cancer cell is measured. The level of expression of a particular marker gene can be measured by “slot blot” hybridization (see Ma et al., (2002) Methods Mol. Biol. 196:139-45). Also, quantitative RT-PCR can be used to determine the expression of marker genes in a cancer cell sample and a normal cell sample. Real-time quantitative PCR can be conveniently accomplished, e.g., using the commercially available ABI PRISMJ 7700 Sequence Detection System (available from PE-Applied Biosystems, Foster City, Calif.) according to manufacturer's instructions. Alternatively, RNA blotting can be used to screen cancer cell samples and normal cell samples for the expression of marker genes. RNA blot analysis is routine in the art (see, e.g., Ausubel, et al., Current Protocols in Molecular Biology, Vol. 1, pp. 4.2.1-4.2.9, John Wiley & Sons, Inc., 1996). Expression levels between cancer cell samples and normal cell samples can be compared using standard statistical techniques known to those of skill in the art (see, e.g., Ma et al., (2002) Methods Mol. Biol. 196:139-45).

The cancer cell sample can be isolated from a human patient by a physician and tested for expression of marker genes using a focused microarray. In addition, the cancer cell sample can be isolated from an organism that develops a tumor or cancer cells including, but not limited to, mouse, rat, horse, pig, guinea pig, or chinchilla. Cell samples can be stored for extended periods prior to testing or tested immediately upon isolation of the cell sample from the subject. Cell samples can be isolated by non-limiting methods such as surgical excision, aspiration from soft tissues such as adipose tissue or lymphatic tissue, biopsy, or removed from the blood. These methods are known to those of skill in the art.

1.2. Capture Probes

A capture probe can be a nucleic acid sequence, which can be a full length sequence, fragments of full length sequences or synthesized oligonucleotides, that binds under physiological conditions to nucleic acids, e.g., by Watson-Crick base pairing (interaction between oligonucleotides and single-stranded nucleic acid) or by any other means including in the case of oligonucleotides binding to RNA, pseudoknot formation. Capture probes can be composed of DNA, RNA, or both. Nucleic acid capture probes are complementary to cDNA or cRNA sequences obtained from pre-messenger RNA, messenger RNA, transfer RNA, heteronuclear RNA (“HnRNA”), ribosomal RNA, bacterial RNA, mitochrondrial RNA or viral RNA.

“Nucleic acid” refers to a polymer comprising two or more nucleotides and includes single-, double-, and triple-stranded polymers. “Nucleotide” refers to both naturally occurring and non-naturally occurring compounds and comprises a heterocyclic base, a sugar, and a linking group, such as a phosphate ester. For example, structural groups may be added to the ribosyl or deoxyribosyl unit of the nucleotide, such as a methyl or allyl group at the 2′-O position or a fluoro group that substitutes for the 2′-O group. The linking group, such as a phosphodiester, of the nucleic acid may be substituted or modified, for example with methyl phosphonates or O-methyl phosphates. Bases and sugars can also be modified, as is known in the art. “Nucleic acid,” for the purposes of this disclosure, also includes “peptide nucleic acids” in which native or modified nucleic acid bases are attached to a polyamide backbone.

The length of a nucleic acid capture probe is less than or equal to the full length of an RNA product generated by a gene sequence so long as the capture probe sequence is complementary to the marker gene sequences and shows less than or equal to 30% homology to other known sequences within the organism being studied. Importantly, nucleotide sequences of between about 50 and about 150 bases in length provide optimal gene expression resolution, while reducing background, non-specific hybridization that occurs with nucleic acid sequences of full length genes (Cheng-Chung Chou et. al. (2004) Nucleic Acids Res. 32(12): e 99). The length of the oligonucleotide can be between about 55 and about 145 bases, between about 60 and about 140 bases, between about 65 and about 135 bases, between about 70 and about 130 bases, and/or between about 75 and about 125 bases. However, sequences greater than about 150 base pairs and less than about 50 base pairs are still effective capture probes and can be used to identify marker genes.

Nucleic acid capture probes can be obtained by any means known in the art. For example, they can be synthetically produced using the Expedite™ Nucleic Acid Synthesizer (Applied Biosystems, Foster City, Calif.) or other similar devices (see, e.g., Applied Biosystems, Foster City, Calif.). Synthetic oligonucleotides also can be produced using methods well known in the art such as maskless photolithography (see, e.g., Nuwaysir et. al. (2002) Gen. Res. 12:1749-1755), phosphoramidite methods (see, e.g., Pan et. al. (2004) Biol. Proc. Online. 6:257-262), H-phosphonate methodology (see, e.g., Agrawal et. al. (1987) Tetrahedron Lett. 28(31): 3539-3542) and phosphite trimester methods (Chou et al. (1983) Biochemistry 22(13):3037-41).

It should be recognized that the capture probes can be attached to linkers such as 3′ amino linkers or 5′ amino linkers without changing the functionality of the capture probes. Also, additional nucleotides can be attached to the 3′ end of a capture probe during nucleic acid synthesis for the purpose of acting as a linker. Generally, linkers can be attached to capture probes to improve the binding efficiency of the capture probe to the target nucleic acid. The procedures used to attach various linker moieties to capture probes are recognized in the art (see, e.g., Steinberg et al. (2004) Biopolymers 73(5):597-605).

Additionally, the capture probes can be modified in a number of ways that would not compromise their ability to hybridize to a particular nucleic acid sequence. Modifications to the nucleic acid structure can include synthetic linkages such as alkylphosphonates, phosphoramidites, carbamates, carbonates, phosphate esters, acetamide, and carboxymethyl esters (see, e.g., Agrawal et. al. (1987) Tetrahedron Lett. 28:3539-3542; Agrawal et. al. (1988) PNAS (USA) 85:7079-7083; Uhlmann et. al. (1990) Chem. Rev. 90:534-583; Agrawal et. al. (1992) Trends Biotechnol. 10:152-158). Additionally, nucleic acid modifications include internucleoside phosphate linkages such as cholesteryl linkages or diamine compounds of varying numbers of carbon residues between the amino groups and terminal ribose. Other modifications of capture probes include changes to the sugar moiety such as arabinose or 3′, 5′ substituted nucleic acids having a sugar attached at its 3′ and 5′ ends through a chemical group other than a hydroxyl group. These modifications can be added to a capture probe sequence without compromising hybridization efficiency (see, e.g., Valoczi et. al., (2004) Nucleic Acids Res. 32(22): e175; Zatsepin et. al., (2004) IUBMB Life. 56(4): 209-214). Therefore, modifications that do not compromise the hybridization efficiency of the capture probe are within the scope of the invention.

1.3. Marker Genes

Marker gene expression is used to identify tumorigenic potential. Marker genes can be obtained by isolation from a cell sample using techniques available to one of ordinary skill in the art (see, e.g., Ausubel et. al., Current Protocols in Molecular Biology, Wiley and Sons, New York, N.Y., 1999). Isolation of nucleic acids from the potentially tumorigenic cell sample allows for the generation of target molecules that can be captured by the capture probes on the surface of a microarray, providing a means for determining the expression level of the marker genes in the potentially tumorigenic cell sample as described below. The marker genes can be isolated from a tissue sample isolated from a human patient. Marker genes can be isolated in the form of RNA transcripts. Methods of RNA isolation are taught in, for example, Ausubel et al., Current Protocols in Molecular Biology, Vol. 1, pp. 4.1.1-4.2.9 and 4.5.1-4.5.3, John Wiley & Sons, Inc., (1993).

Useful marker genes detected to determine the existence of cancer include cathepsin β (gi# 22538429) 942-1010 bp of cds; BIP (gi# 6470149) 1631-1698 bp of cds; annexin-1/p-40 (gi# 4502100) 823-890 bp of cds; Heat shock 70 kDa protein 8 (HSC70) (gi# 5729876) 1451-1518 bp of cds; Heat shock 70 kDa protein 8 (HSC70) (gi# 5729876) 1645-1712 bp of cds; galectin-1 (gi# 6006015) 341-408 bp of cds; Heat shock protein 27 (HSP27) (gi# 4996892) 61-128 bp of cds; ubiquitin C-term hydrolase isozyme L1 (UCHL-1) (gi# 18558293) 213-280 bp of cds; ubiquitin C-term hydrolase isozyme L1 (UCHL-1) (gi# 18558293) 471-538 bp of cds; ATP synthase β (gi# 179280) 1033-1200 bp of cds; prosolin (gi# 13518023) 351-418 bp of cds; thioredoxine peroxidase 1 (gi# 440307) 529-597 bp of cds; 0-tubulin (gi# 3387928) 400-468 bp of cds; guanine nucleotide binding protein, β polypeptide 3 (GNBP β3) (gi# 183412) 350-398 bp of cds; MB-COMT (gi# 6466451) 101-168 bp of cds; EZRIN (gi# 21614498) 1011-1078 bp of cds; KAP-1 (gi# 1699026) 1-68 bp of cds; UMP-CMP kinase (gi# 5730475) 391-458 bp of cds; alternative splicing factor (ASF-2) (gi# 179073) 811-878 bp of cds; pyrophosphatase inorganic (gi# 12735403) 533-600 bp of cds; GST-π α chain (gi# 31947) 565-633 bp of cd synthase D (gi# 5453558) 213-280 bp of cds; chromobox homolog 3 (CBX3) (gi# 15082257) 31-98 bp of cds; protein disulfide isomerase precursor (PDI) (gi# 20070124) 543-610 bp of cds; dimethylarginine dimethylaminohydrolase 1 (DADEH1) (gi# 6912327) 399-456 bp of cds; dimethylarginine dimethylaminohydrolase 1 (DADEH1) (gi# 6912327) 651-718 bp of cds; Elongation factor 2 (EF2) (gi# 181968) 833-900 bp of cds; α-enolase (gi# 2661038) 943-1010 bp of cds; eukaryotic translation factor 3 subunit 2 (ETF3-subunit 2) (gi# 4503512) 833-900 bp of cds; heterogenous nuclear ribonucleoprotein F (HnRNP) (gi# 14141150) 771-838 bp of cds; tropomyosin 2β (gi# 20070122) 550-617 bp of cds; eukaryotic translation initiator factor 4B (EIF 4B) (gi# 4503532) 901-968 bp of cds; hepatoma derived growth factor (gi# 4758515) 393-460 bp of cds; keratin type II cytoskeletal (gi# 12737278) 1171-1238 bp of cds; prohibitin (gi# 6031190) 713-780 bp of cds; solute carrier family 9 isoform 3 regulatory factor 1 (s1c9A3R1) (gi# 4759139) 631-738 bp of cds; 5C5-2 (gi# 4324471) 141-208 bp of cds; protein disulfide isomerase ER-60 precursor (PDI-ER60) (gi# 1208427) 833-900 bp of cds; β-spectrin (gi# 338439) 3100 -3168 bp of cds; β-spectrin (gi# 338439) 4000-4068 bp of cds; Superoxide dismutase (SOD) (gi# 4507148) 391-458 bp of cds; caspase recruitment domain protein 14 (gi# 13653996) 895-968 bp of cds; N-ethylmaleimide-sensitive factor attachment protein γ (NEM-sensitive factor attachment protein γ) (gi# 4505330) 732-800 bp of cds; fatty acid synthase (FAS) (gi# 4758341) 1-68 bp of cds; fatty acid synthase (FAS) (gi# 4758341) 7233-7300 bp of cds; triosephosphate isomerase (TPI) (gi# 339840) 400-467 bp of cds; Rad23 homolog β (gi# 19924138) 900-968 bp of cds; L-Plastin (gi# 16307447) 1600-1668 bp of cds; α-tubulin (gi# 3420928) 1288-1356 bp of cds; fatty acid binding protein, epidermal (E-FABP) (gi# 4557580) 1-68 bp of cds; fatty acid binding protein, epidermal (E-FABP) (gi# 4557580) 341-408 bp of cds; “similar to stratifin” (gi# 16306736) 314-382 bp of cds; cathepsin D (gi# 18577791) 411-478 bp of cds; p16INK4a (gi# 16753086) 1-68 bp of cds; p16INK4a (gi# 16753086) 50-118 bp of cds; adenine phosphoribosyltransferase (APRT) (gi# 4502170) 100-168 bp of cds; calumenin (gi# 14718452) 880-943 bp of cds; ACRABP-II (gi# 6382069) 481-548 bp of cds; keratin 19 (gi# 40217850) 141-208 bp of cds; c-erb/HER-2/neu (gi# 4758297) 1981-2048 bp of cds; MYL16 (gi# 17986259) 252-319 bp of cds; interleukine 18 precursor (gi# 14210476) 431-498 bp of cds; cytokeratin 18 (gi# 40354194) 1181-1248 bp of cds; cytokeratin 7 (gi# 3008955) 1461-1528 bp of cds; prostatin (gi# 21536453) 891-958 bp of cds; estrogen receptor α (gi# 4503602) 2041-2108 bp of cds; 14-3-3 eta chain (gi# 21464102) 301-368 bp cds.

The marker genes derived from the cell sample can be further utilized to produce the targets-of-interest (herein termed “nucleic acid probes”) for the capture probes. As used herein, a “nucleic acid probe” is defined as a nucleic acid capable of binding to a nucleic acid of complementary sequence through one or more types of chemical bonds, usually through complementary base pairing, usually through hydrogen bond formation. As used herein, a nucleic acid probe may include natural (i.e. A, G, U, C, or T) or modified (7-deazaguanosine, inosine, etc.) bases. In addition, a linkage other than a phosphodiester bond may join the bases in probes, so long as it does not interfere with hybridization. Thus, nucleic acid probes may be peptide nucleic acids in which the constituent bases are joined by peptide bonds rather than phosphodiester linkages. The nucleic acid probes may be prepared by converting the RNA to cDNA using known methods (see, e.g., Ausubel et. al., Current Protocols in Molecular Biology Wiley 1999). The probes can also be cRNA (see, e.g., Park et. al., (2004) Biochem. Biophys. Res. Commun. 325(4):1346-52).

Nucleic acid probes can be produced from synthetic methods such as phosphoramidite methods, H-phosphonate methodology, and phosphite triester methods. Nucleic acid probes can also be produced by PCR methods. Such methods produce cDNA and cRNA sequences complementary to the mRNA.

The nucleic acid probes can be detectably labeled. As used herein, “detectably labeled” means that a probe is operably linked to a moiety that is detectable. By “operably linked” is meant that the moiety is attached to the probe by either a covalent or non-covalent (e.g., ionic) bond. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y., 1999).

According to the invention, a “detectable label” is a moiety that can be sensed. Such labels can be, without limitation, fluorophores (e.g., fluorescein (FITC), phycoerythrin, rhodamine), chemical dyes, or compounds that are radioactive, chemoluminescent, magnetic, paramagnetic, promagnetic, or enzymes that yield a product that may be colored, chemoluminescent, or magnetic. The signal is detectable by any suitable means, including spectroscopic, photochemical, biochemical, immunochemical, electrical, optical or chemical means. In certain cases, the signal is detectable by two or more means. In certain embodiments, nucleic acid labels include fluorescent dyes, radiolabels, and chemiluminescent labels, which are examples that are not intended to limit the scope of the invention (see, e.g., Yu, et al., (1994) Nucleic Acids Res. 22(16): 3226-3232; Zhu, et al., (1994) Nucleic Acids Res. 22(16): 3418-3422).

For example, nucleotides of nucleic acid probes may be conjugated to Cy5/Cy3 fluorescent dyes. These dyes are frequently used in the art (see, e.g., Yang et al., (2005) Clin. Cancer Res. 11(2 Pt 1):612-20). The fluorescent labels can be selected from a variety of structural classes, including the non-limiting examples such as 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein).

Other useful dyes are chemiluminescent dyes and can include, without limitation, biotin conjugated DNA nucleotides and biotin conjugated RNA nucleotides. Labeling of nucleic acid probes can be accomplished by any means known in the art, e.g., CyScribe™ First Strand cDNA Labeling Kit (#RPN6200, Amersham Biosciences, Piscataway, N.J.).

The label can be added to the target nucleic acid(s) prior to, or after the hybridization. So called “direct labels” are detectable labels that are directly attached to, or incorporated into, the target nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore binds the biotin bearing hybrid duplexes providing a label that is easily detected. (see, e.g., Laboratory Techniques in Biochemistry and Molecular Biology, Vol. 24: Hybridization With Nucleic Acid Probes, P. Tijssen, ed. Elsevier, N.Y., (1993)).

1.4. Cancer Prediction Analysis

It should be recognized that statistical analysis of the levels of expression of marker genes in a cell sample does not require a particular algorithm or set of particular algorithms. An algorithm can be used in the present invention so long as it can discriminate between statistically significant and statistically insignificant differences in the levels of expression of marker genes in a cell sample as compared to the levels of expression of the same marker genes in a normal cell sample of the same tissue type.

Class prediction algorithms can be utilized to differentiate between the levels of expression of marker genes in a cell sample and the levels of expression of marker genes in a normal cell sample (see Vapnik, The Nature of Statistical Learning Theory, Springer Publishing, 1995). Exemplary, non-limiting algorithms include, but are not limited to, compound covariate predictor, diagonal linear discriminant analysis, nearest neighbor predictor, nearest centroid predictor, and support vector machine predictor (see Simon et al., Design and Analysis of DNA Microarray Investigations: An Artificial Intelligence Milestone., Springer Publishing, 2003). These statistical tests are well known in the art, and can be applied to microarray data or data generated using other gene expression determination techniques such as RNA dot blotting, Northern blotting, and RT-PCR (see, e.g., Pomeroy et al., (2002) Nature 415: 436-442; Dudoit and Fridlyand (2002) Gen. Biol. 3(7): Research0036; Furey et al. (2000) Bioinform. 16(10): 906-14).

Cancer diagnoses can be performed by comparing the levels of expression for a marker gene or a set of marker genes in a neoplastic cell sample to the levels of expression for a marker gene or a set of marker genes in a normal cell sample of the same tissue type. Alternatively, the level of expression for a marker gene or a set of marker genes in a cell sample is compared to a reference pool of RNA that represents the level of expression for a marker gene or a set of marker genes in a normal population (herein termed “training set”). The training set also includes the data for a population that has a known tumor or class of tumors. This data represents the average level of expression that has been determined for the neoplastic cells isolated from the tumor or class of tumors. It also has data related to the average level of expression for a marker gene or set of marker genes for normal cells of the same cell type within a population. In these embodiments, the algorithm compares newly generated expression data for a particular marker gene or set of marker genes from a cell sample isolated from a patient containing potentially neoplastic cells to the levels of expression for the same marker gene or set of marker genes in the training set. The algorithm determines whether a cell sample is neoplastic or normal by aligning the level of expression for a marker gene or set of marker genes with the appropriate group in the training set.

In some embodiments, an increased level of expression in the cell sample indicates that cancer cells exist in the cell sample. Marker genes showing increased levels of expression include, but are not limited to, HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, ATP synthase δ, PDI/ER-60 precursor, ATP synthase β, prostasin, cathepsin β, FAS, HSCP60, topoisomerase IIα, PCNA, ezrin, PDI, cathepsin D, A-CRABP II, HSC70, rad 23 homolog β, ETF3 subunit 2β, proteosome B1 subunit proprotein, β-tubulin, s1c9a3r1, prosolin, HSP60, HER-2, L-plastin, estrogen receptor α, HSP27, thioredoxine peroxidase I, calumenin, 14-3-3 eta chain, Ki 67, MRP1, “similar to stratifin,” UCHL-1, mammaglobin 2, cellular RNA binding protein, and annexin I. However, a decrease in expression of certain marker genes such as FABP4 indicates that the patient cell sample is potentially neoplastic. The algorithm makes the class prediction based upon the overall levels of expression found in the cell sample as compared to the levels of expression in the training set. It should be noted that, in some instances, one marker gene can be used to classify a gene as either neoplastic or normal. Two or more marker genes can also be used to properly classify a cell sample as neoplastic or normal. In particular, three marker genes can be used for classification purposes. Four marker genes can be used to identify neoplastic cells within a cell sample. Five marker genes can be used to identify neoplastic cells in a cell sample. Furthermore, six or more marker genes can be used to properly classify cell samples into either the neoplastic cell class or the non-neoplastic cell class.

The type of analysis detailed above compares the level of expression for the marker gene(s) in the cell sample to a training set containing reference pools of RNA that are representative of a normal population and a neoplastic population. In certain embodiments, the training set can be obtained with kits that can be used to determine the level of expression of marker gene(s) in a patient cell sample. Alternatively, an investigator can generate new training sets using RNA reference pools that can be obtained from commercial sources such as Asterand, Inc. (Detroit, Mich.). Comparisons between the training sets and the cell samples are performed using standard statistical techniques that are well known in the art, and include, but are not limited to, the ArrayStat 1.0 program (Imaging Research, Inc.). Statistically significant increased levels of expression in the cell sample of marker gene(s) indicate that the cell sample contains a cancer cell or cells with tumorigenic potential. Also, standard statistical techniques such as the Student T test are well known in the art, and can be used to determine statistically significant differences in the levels of expression for marker genes in a patient cell sample (see, e.g., Yagi et al. (2003) Blood 102(5): 1849-56). In particular, the Student T test is used to identify statistically significant changes in expression using microarray analysis (see, e.g., Yagi et al. (2003) Blood 102(5): 1849-56).

To demonstrate the methods according to the invention and the focused microarray, focused microarrays were prepared as described below and tested using the methods described below for their ability to diagnose cancer in cell samples isolated from human subjects suffering from breast or ovarian cancers.

The oligonucleotides tested on the nucleic acid focused microarray have been described above. Oligonucleotides attached to the focused microarray were designed so as to have an overall thermal melting point of 76.69±3.92° C. at a sodium concentration of 50 mM. Normalization of signal was performed using Arabidopsis thaliana chlorophyll synthetase G4 positive control DNA. Statistical analysis was performed using a log transformation of the ratio data on all experiments, and a Student T test was used to determine statistically significant results for two independent conditions using a proportional model without offsets at a p<0.05 threshold. A difference in expression level is found when the ratio of Cy5 to Cy3 is greater than 2.0. Statistically significant differences in expression between samples were found if the p value was lower than 0.05.

To test the focused microarray's capacity to determine increased expression of a nucleic acid marker gene in a potential cancer cell, capture probes were disposed on a microarray. The sequences represented regions of within each marker gene that had homologies to other genes of less than 30%. The capture probes consisted of sequence lengths of 68 bases. Thus, hybridizations between capture probes and marker gene targets would be specific, and uniform hybridization was expected between capture probes and specific targets. By maintaining the average hybridization temperature within a limited range amongst capture probes, the clinician is able to obtain similar intensity results between spots on the chip.

Sample materials were obtained from Asterand, Inc. (Detroit, Mich.), Cytomix LLC (Lexington, Mass.), and Biochain Institute, Inc. (Hayward, Calif.). The clinical characteristics of the normal individuals and the breast cancer patients are shown in Tables 1 and 2.

TABLE 1
Clinical Characteristics of Breast Cancer Patients

| | Age |
|---|---|
| | 52.35443 ± 12.00538 |
| | Number of Patients |
| Ethnicity | |
| Caucasian | 59 |
| Asian | 20 |
| Unknown | 1 |
| Diagnosis | |
| Infiltrating Ductal Carcinoma | 60 |
| Invasive Ductal Carcinoma | 20 |

TABLE 2
Clinical Characteristics of Ovarian Cancer Patients

| | Age |
|---|---|
| | 54.125 ± 10.4378 |
| | Number of Patients |
| Ethnicity | |
| Caucasian | 45 |
| Asian | 11 |
| Unknown | 1 |
| Diagnosis | |
| Serous Adenocarcinoma | 37 |
| Mucinous Adenocarcinoma | 2 |
| Endometrioid Carcinoma | 7 |
| Clear Cell Carcinoma | 3 |
| Ovarian Carcinoma | 8 |

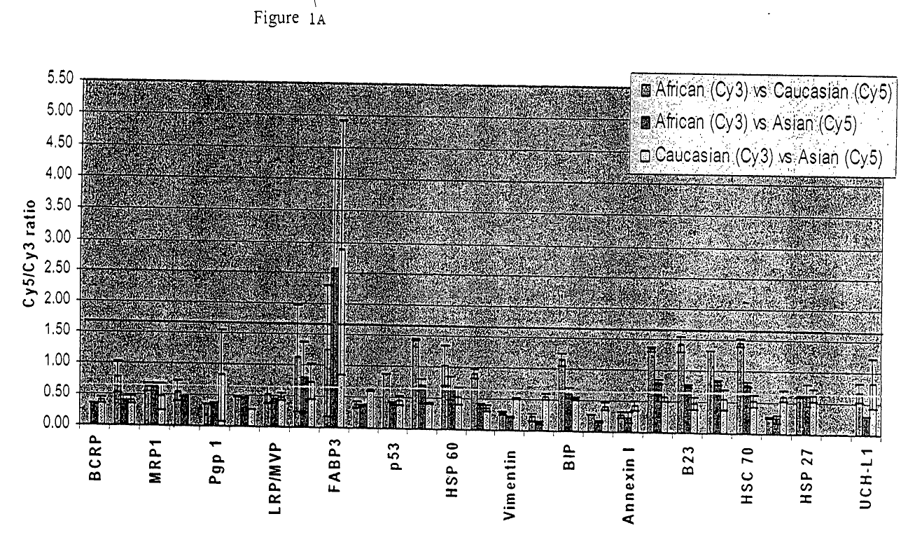

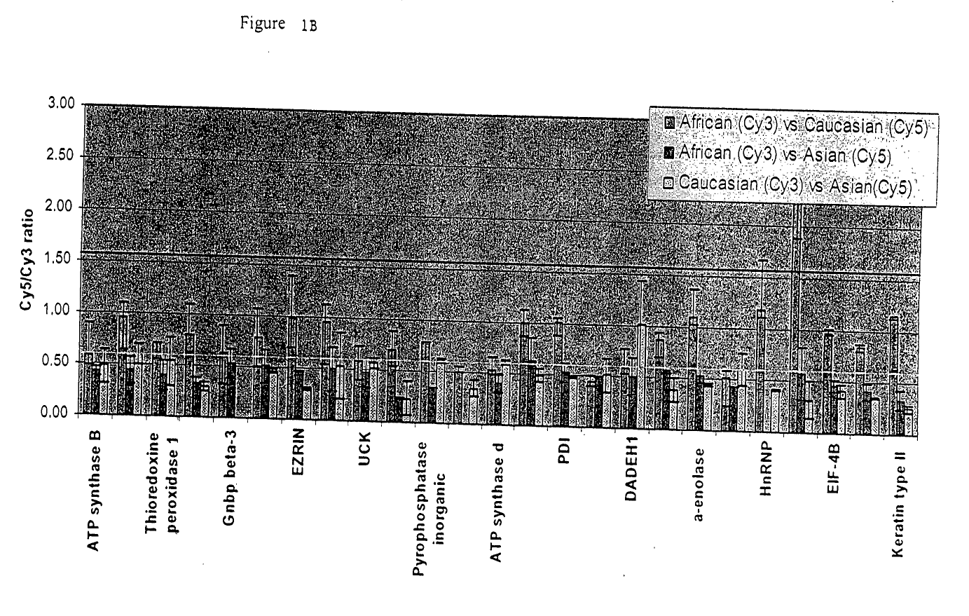

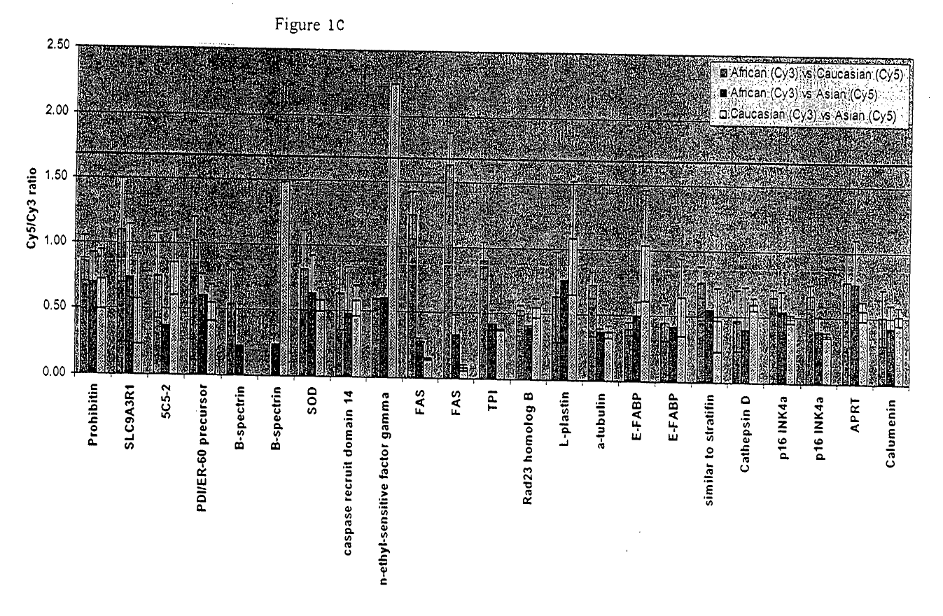

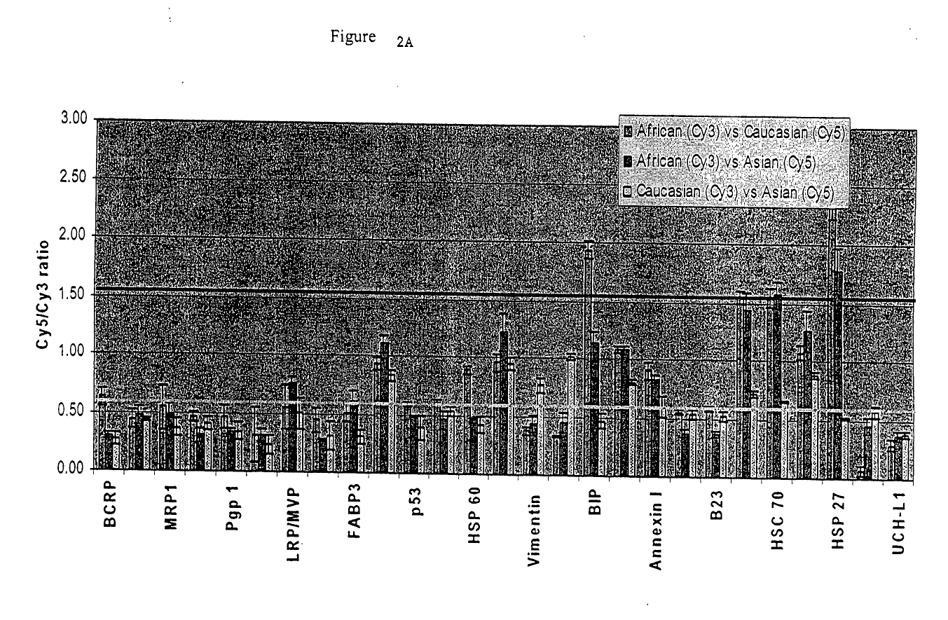

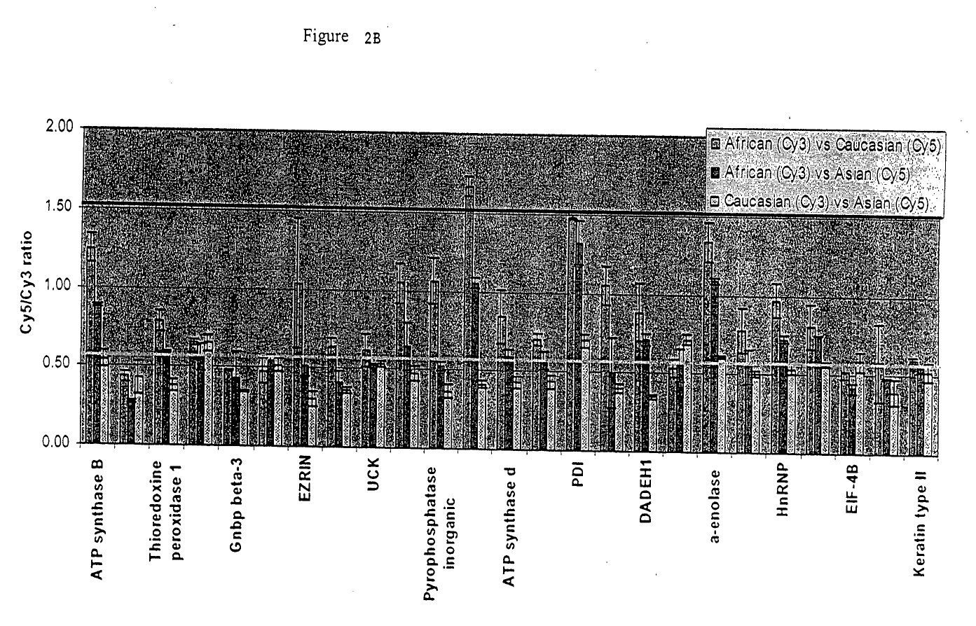

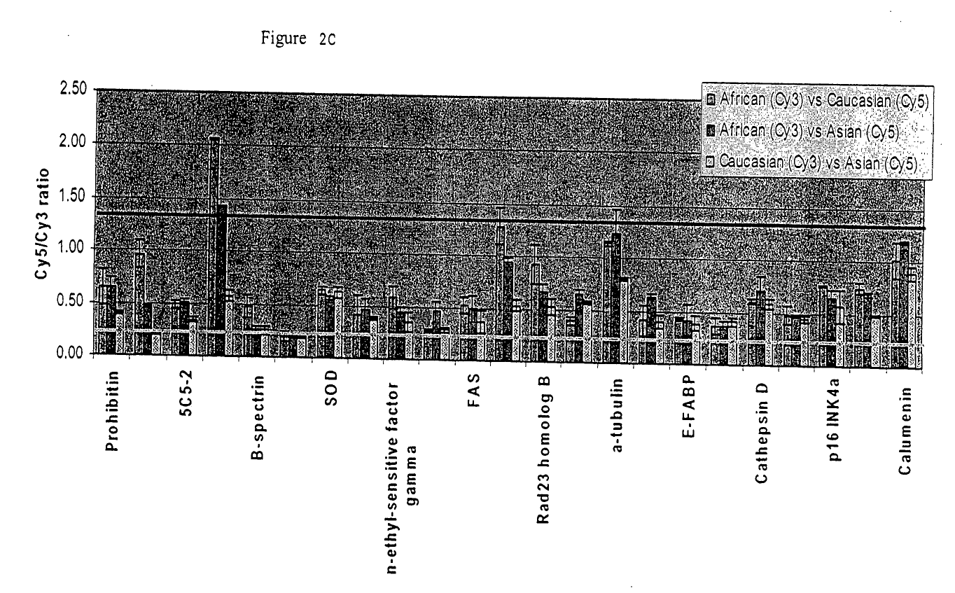

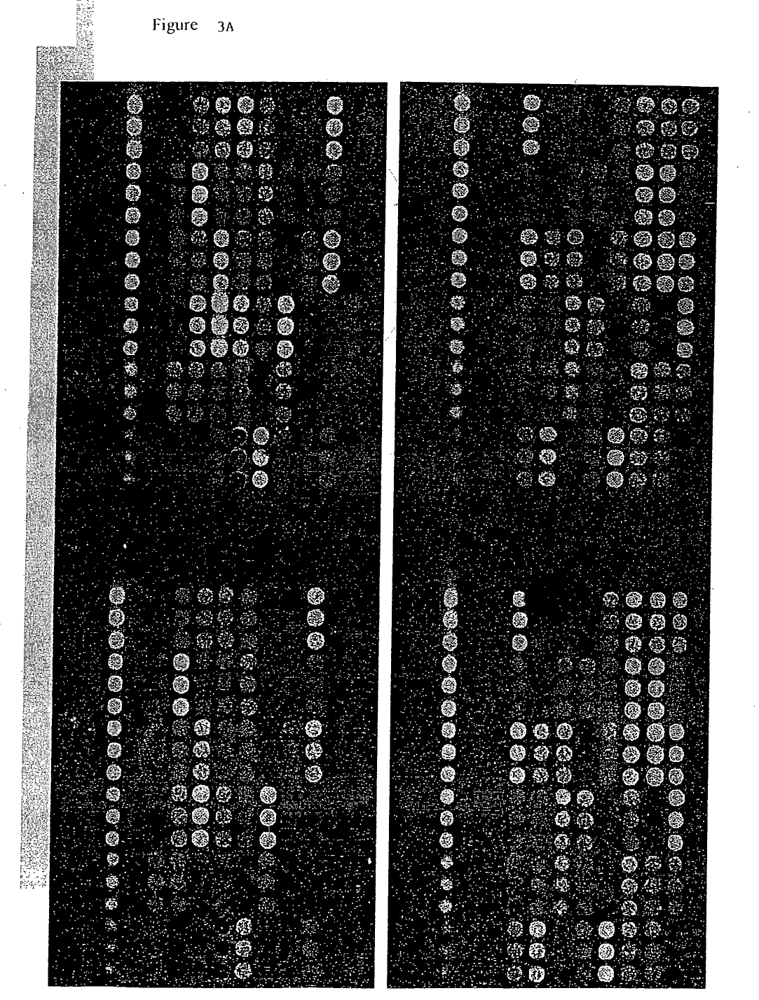

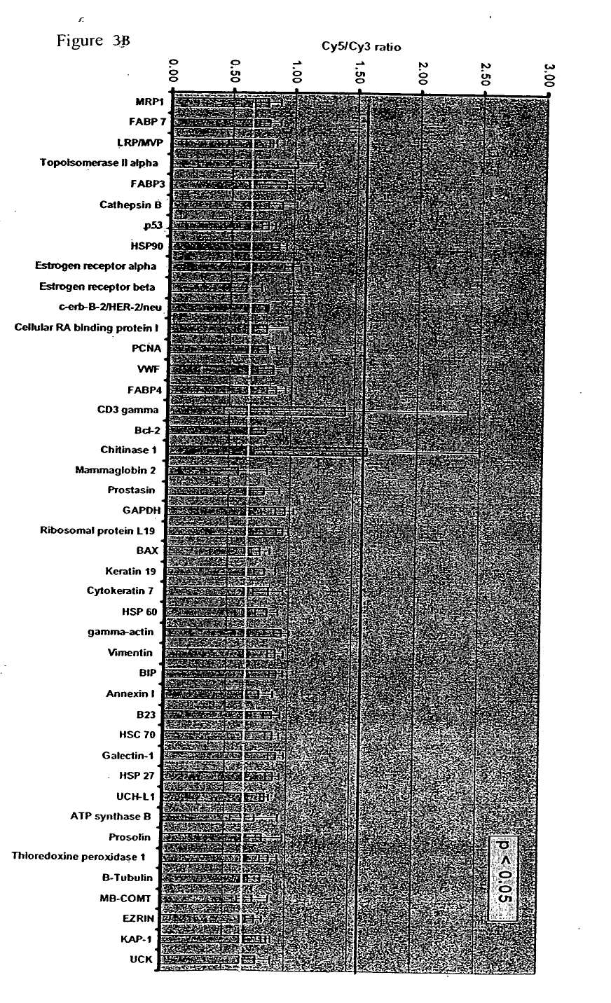

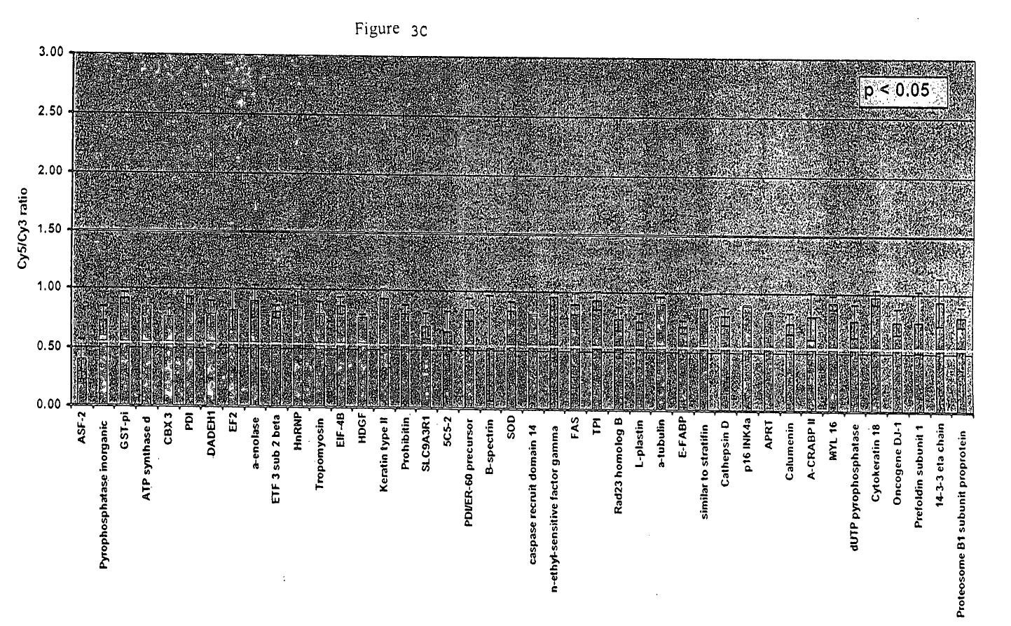

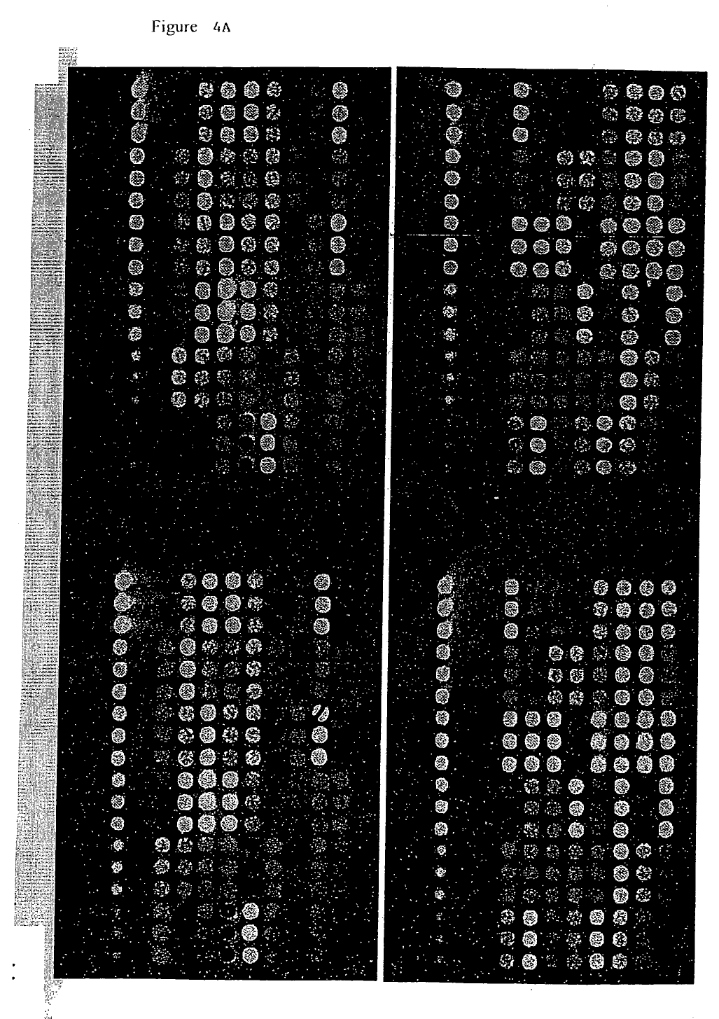

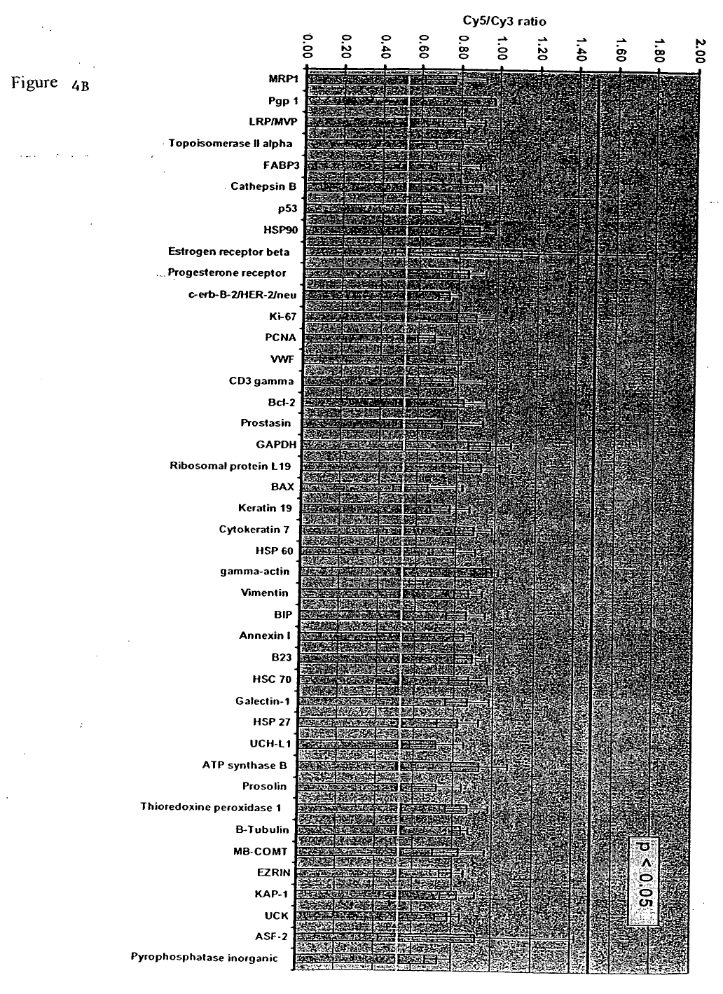

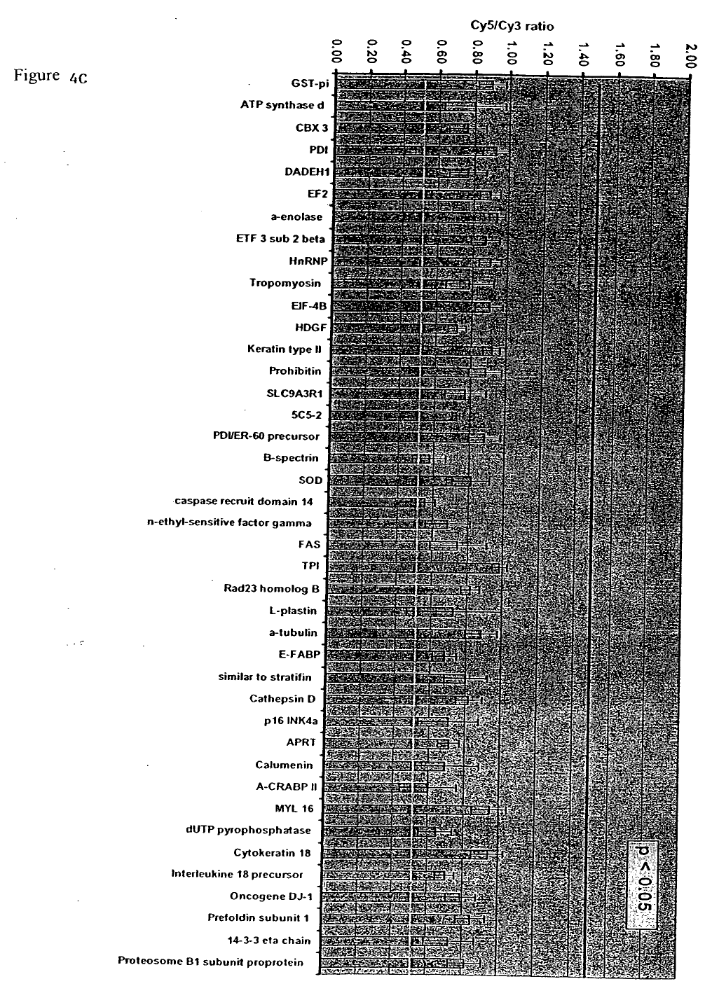

The effect of ethnicity on marker gene expression was determined for the marker genes detected by the focused microarray. In normal breast tissue, there were no statistically significant differences observed between the different ethnicities for the majority of marker genes detected by the microarray (FIG. 1a-c). Cy5/Cy3 ratios were between 0.5 and 1.5 for the majority of the marker genes, which is an insignificant result. Similar results were obtained for ovarian normal samples (FIG. 2a-c). Furthermore, good coverage of the microarray was obtained during the experimental procedure (FIG. 3a-c and FIG. 4a-c). Spot intensity remained similar between microarray chips in each experiment, which indicated that the proper distribution of nucleic acid probes was occurring on the chips. Proper spot coverage was necessary to establish that expression determinations using the invention were due to actual levels of expression of the marker genes, and not inappropriate surface coverage of the probes.

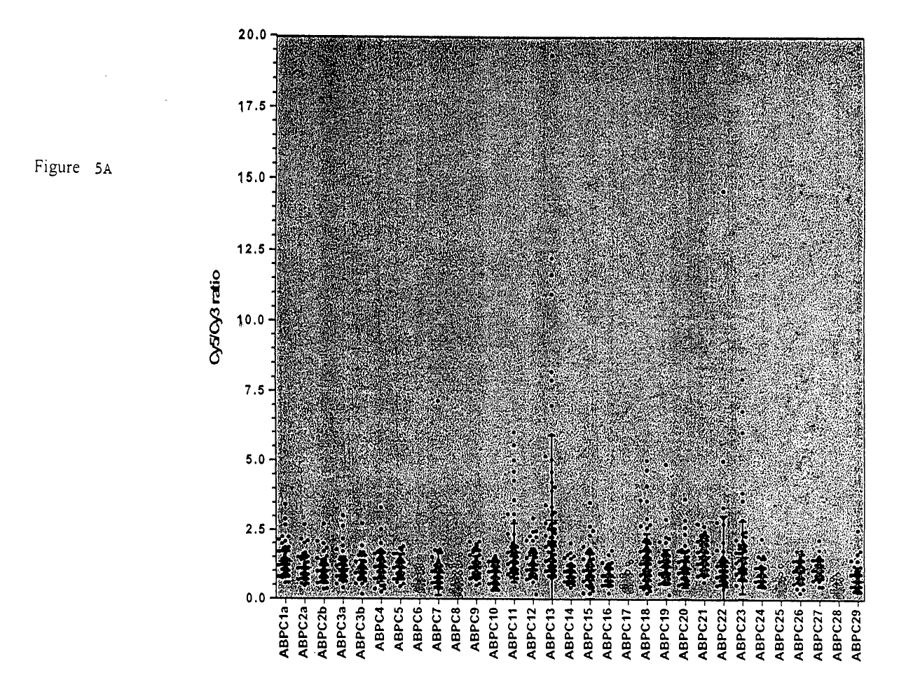

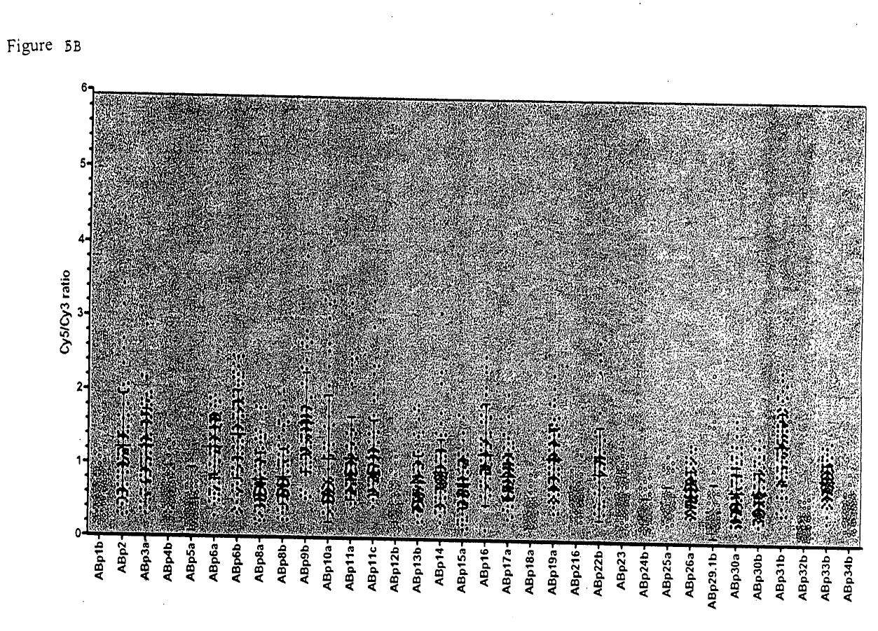

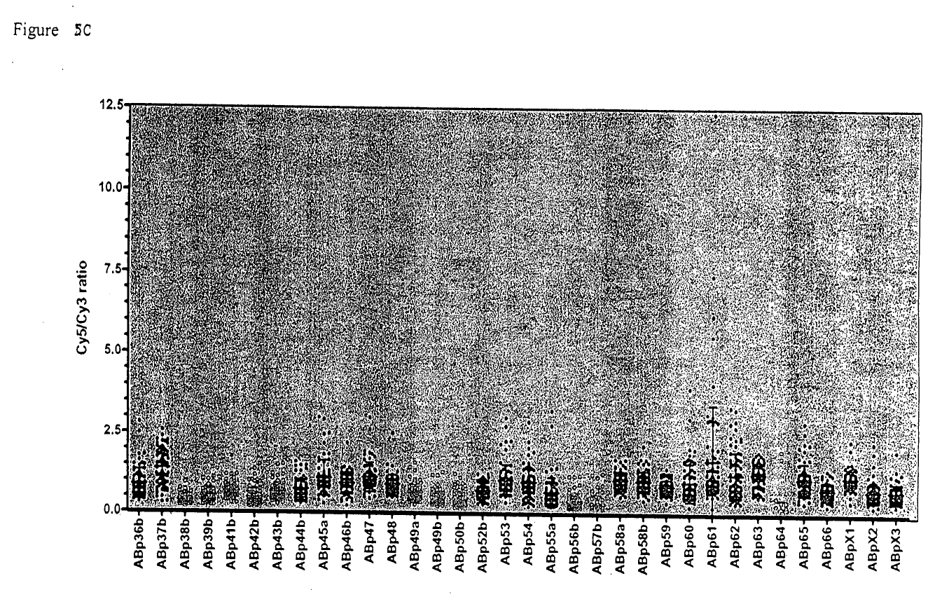

To test the focused microarray against a patient population suffering from ovarian cancer, a pool of RNA was isolated that represented normal ovarian patients and a pool of RNA was isolated from ovarian cancer patients. These pools were used to generate labeled nucleic acid probes for hybridization to the capture probes attached to the microarray. Also, a reference pool was established using RNA from a commercial supplier (Asterand, Inc., Detroit, Mich.), which was compared to the ovarian normal patient and the ovarian cancer patient RNA pools. As shown in FIG. 4a-c, the ovarian normal pool showed decreased expression in approximately 30% of the marker genes on the focused microarray as compared to the reference RNA pool. One marker gene, UMP-CMP kinase, showed increased expression in the study. FIGS. 5a-c show the results of the ovarian cancer patient RNA pool comparison to the reference normal RNA pool. In that study, approximately 30% of the marker genes on the focused microarray showed increased expression in the ovarian cancer pool, while vimentin showed a decrease in expression in the ovarian cancer pool. Therefore, it is apparent that a normal ovarian cell sample has a significantly different expression profile to an ovarian cancer cell sample with respect to the marker genes analyzed using the focused microarray.

The results for the ovarian normal RNA pool were compared to the results for the ovarian cancer RNA pool to determine the marker genes that showed the greatest difference in expression between the two samples. The study identified 27 marker genes that showed increased levels of expression in the ovarian cancer pool of 3 times over the levels of expression in the ovarian normal pool. The results of the comparison are shown in Table 3.

TABLE 3

| | Normal Ovarian | Ovarian Tumor | |
|---|---|---|---|
| t-Value | Expression | Expression | Marker Gene |
| −23 | 0.34 | 1.799 | HDGF |
| −21.04 | 0.099 | 1.376 | cytokeratin 18 |
| −20.31 | 0.24 | 1.536 | α-enolase |
| −19.17 | 0.39 | 2.021 | GAPDH |
| −18.96 | 0.397 | 1.612 | GST-π |
| −18.73 | 0.41 | 2.65 | TPI |
| −18.67 | 0.57 | 2.26 | 5C5-2 |
| −18.24 | 0.619 | 1.967 | prohibitin |
| −17.26 | 0.356 | 2.062 | keratin 19 |
| −16.8 | 0.585 | 7.504 | cytokeratin 7 |
| −15.5 | 0.578 | 2.611 | pyrophosphatase |
| −15.42 | 0.562 | 2.008 | BIP |
| −15.35 | 0.683 | 2.313 | CBX3 |
| −14.83 | 0.669 | 2.213 | PDI/ER-60 |
| −14.59 | 0.415 | 1.73 | ATP synthase β |
| −14.4 | 0.753 | 3.38 | prostasin |
| −14.39 | 0.42 | 1.925 | cathepsin β |
| −14.37 | 0.43 | 1.376 | FAS |
| −13.74 | 0.524 | 1.814 | HSCP60 |
| −13.42 | 0.563 | 1.71 | topoisomerase IIα |
| −13.37 | 0.579 | 1.757 | PCNA |
| −13.22 | 0.36 | 1.273 | ezrin |
| −12.46 | 0.354 | 1.255 | PDI |
| −11.62 | 0.208 | 0.836 | cathepsin D |
| −10.12 | 1.269 | 6.853 | A-CRABP II |
| −7.76 | 0.4 | 1.248 | annexin I |

The experiment established that several marker genes had 4 to 6 fold-increased expression in ovarian cancer cells than in normal cancer cells. Such differences in expression identified these marker genes as being particularly useful diagnostic markers for the development of cancer in ovarian tissues. Table 4 shows the marker genes that have four fold, five fold, and six fold differences in levels of expression between ovarian cancer cell samples and normal ovarian cell samples.

TABLE 4

| | Normal Ovarian | Ovarian Tumor | |
|---|---|---|---|
| t-Value | Expression | Expression | Marker Gene |
| −23 | 0.34 | 1.799 | HDGF |
| −21.04 | 0.099 | 1.376 | cytokeratin 18 |
| −20.31 | 0.24 | 1.536 | α-enolase |
| −19.17 | 0.39 | 2.021 | GAPDH |
| −18.96 | 0.397 | 1.612 | GST-π |
| −18.73 | 0.41 | 2.65 | TPI |
| −18.67 | 0.57 | 2.26 | 5C5-2 |
| −18.24 | 0.619 | 1.967 | prohibitin |
| −17.26 | 0.356 | 2.062 | keratin 19 |
| −16.8 | 0.585 | 7.504 | cytokeratin 7 |
| −15.5 | 0.578 | 2.611 | pyrophosphatase |
| −14.59 | 0.415 | 1.73 | ATP synthase β |
| −14.4 | 0.753 | 3.38 | prostasin |
| −14.39 | 0.42 | 1.925 | cathepsin β |

In addition, the results for the ovarian normal RNA pool were compared to the results for the breast cancer RNA pool to determine the marker genes that showed the greatest difference in expression between the two samples. The study identified 34 marker genes that showed increased levels of expression in the ovarian cancer pool of two times over the levels of expression in the ovarian normal pool. The results of the comparison are shown in Table 5.

TABLE 5

| | Normal Breast | Breast Tumor | |
|---|---|---|---|
| t-Value | Expression | Expression | Marker Gene |
| −12.36 | 0.799 | 2.997 | cathepsin D |
| −10.63 | 0.852 | 2.027 | HnRNP |
| −9.65 | 0.878 | 2.075 | PDI/ER-60 |
| −9.47 | 0.986 | 2.727 | ezrin |
| −9.2 | 1.002 | 3.99 | keratin 19 |
| −9.12 | 0.797 | 2.233 | cathepsin β |
| −8.96 | 0.975 | 2.382 | CBX3 |
| −8.96 | 0.672 | 1.458 | HSC70 |
| −8.95 | 1.106 | 2.471 | β-tubulin |
| −8.76 | 0.942 | 2.292 | PDI |
| −8.48 | 0.637 | 1.356 | BIP |
| −8.31 | 1.216 | 4.65 | slc9a3r1 |
| −8.18 | 0.76 | 1.918 | TPI |
| −8.11 | 0.679 | 1.657 | HDGF |
| −7.56 | 1.085 | 2.284 | thioredoxine |
| | | | peroxidase I |
| −7.39 | 1.022 | 2.343 | PCNA |
| −7.2 | 0.928 | 2.434 | prosolin |
| −7.14 | 0.863 | 1.82 | 5C5-2 |
| −7.06 | 0.879 | 1.804 | 14-3-3 eta chain |
| −6.86 | 0.838 | 2.17 | topoisomerase IIα |
| −6.68 | 0.596 | 1.498 | prostasin |
| −6.6 | 1.075 | 2.345 | calumenin |
| −6.24 | 0.871 | 1.809 | GAPDH |
| −5.93 | 0.585 | 1.239 | HSCP60 |
| −5.73 | 0.943 | 1.922 | prohibitin |
| −5.59 | 1.172 | 4.582 | HER-2 |
| −5.45 | 1.93 | 5.927 | A-CRABP II |
| −5.36 | 0.767 | 1.58 | cytokeratin 18 |
| −5.29 | 1.059 | 2.228 | L-plastin |
| −5.15 | 1.224 | 3.513 | estrogen receptor α |
| −4.73 | 0.358 | 0.835 | HSP27 |
| 6.2 | 1.387 | 0.573 | annexin I |
| 7.7 | 0.933 | 0.414 | VWF |
| 8.07 | 0.366 | 0.05 | FABP4 |

Table 6 shows the genes that are expressed at levels greater than or equal to three times in breast cancer cells as compared to normal cells.

TABLE 6

| | Normal Breast | Breast Tumor | |
|---|---|---|---|
| t-Value | Expression | Expression | Marker Gene |
| −13.16 | 0.789 | 3.124 | cathepsin D |
| −10.72 | 0.936 | 2.841 | ezrin |
| −10.26 | 0.93 | 4.224 | keratin 19 |
| −8.76 | 1.206 | 4.957 | slc9a3r1 |
| −5.97 | 1.837 | 6.375 | A-CRABP II |
| −5.81 | 1.154 | 4.953 | HER-2 |
| −5.37 | 1.232 | 3.797 | estrogen receptor α |
| 9.52 | 0.425 | 0.045 | FABP4 |

After determining the marker genes that were overexpressed in cancer cell samples, each cancer patient's expression profile and normal subject's expression profile for particular sets of overexpressed marker genes was plotted onto graphs. The graphs represented the standard deviation in the average levels of expression for particular sets of marker gene(s) in a group of patients. A proper classification indicated that the focused microarray identified marker genes that can be used to discriminate between a cell sample containing cancerous cells and cell sample containing solely normal cells. As shown in FIG. 6, cell samples from ovarian cancer patients and normal ovarian subjects were tested using the marker genes cytokeratin 18, cytokeratin 7, TPI, and α-enolase. The average expression for the four marker genes was determined for each patient, and plotted onto the graph. The ovarian cancer group exhibited six times higher average levels of expression for the set of marker genes than their normal ovarian counterparts (FIG. 6). In addition, only one ovarian cancer patient's average level of expression for the marker genes was shown to overlap with any normal ovarian subject's level of expression. The classification algorithms were able to properly classify ovarian cell samples with nearly 100% accuracy.

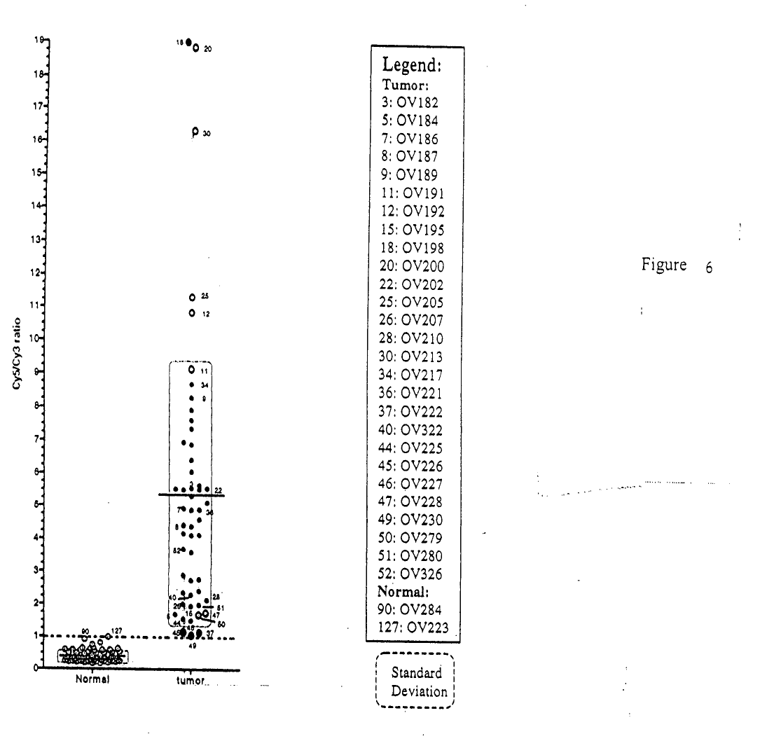

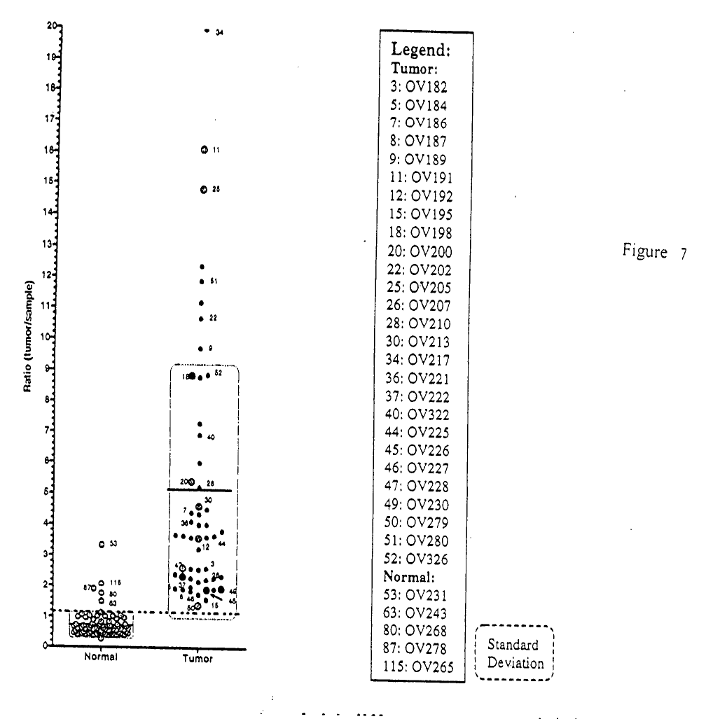

The average levels of expression for the marker genes HDGF, GAPDH, keratin 19, and A-CRABP II were determined in ovarian cancer patients and normal ovarian subjects. The average expression levels of the four marker genes was determined and plotted as in FIG. 6. These four marker genes showed a consistent five times increased level of expression in the ovarian cancer patients as compared to the normal ovarian subject (FIG. 7). There were five normal ovarian subjects that showed expression levels for the four marker genes that overlapped with the ovarian cancer patient group. Although these five subjects showed elevated levels of expression for the marker genes, the test properly classified a cell sample as either ovarian cancer or normal in greater than 95% of cases.

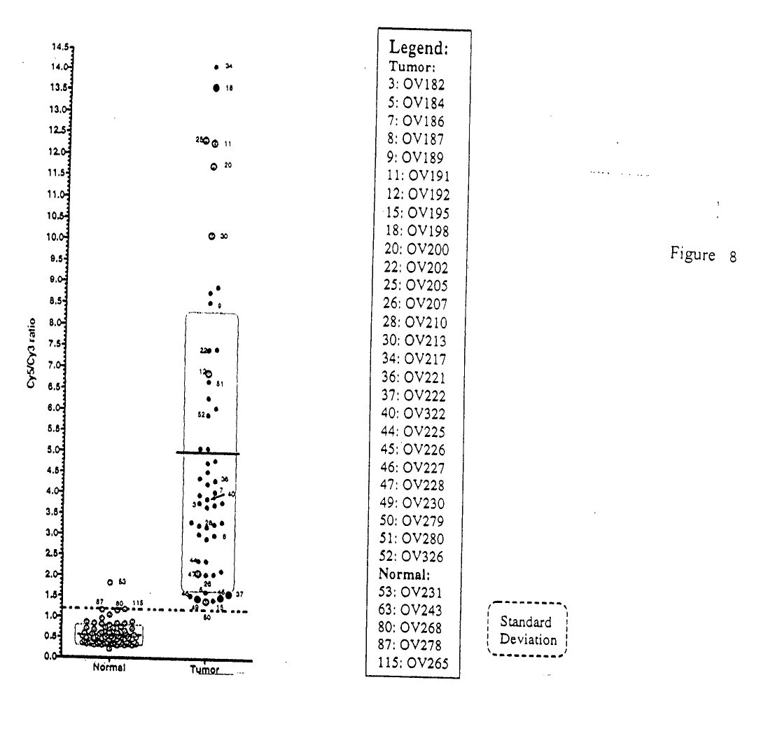

When all eight marker genes were analyzed using the classification algorithms, individual cell samples were properly classified into the ovarian cancer group or the normal ovarian group in greater than 99% of the cases (FIG. 8). One normal ovarian subject showed average levels of expression for the eight marker genes that were within the low range of the ovarian cancer group. The differences between the ovarian cell samples and the normal ovarian cell samples were large enough for classification of the cell sample using the classification algorithms with the training sets applicable to the ovarian cancer. Proper classification was performed using the classification algorithms detailed above.

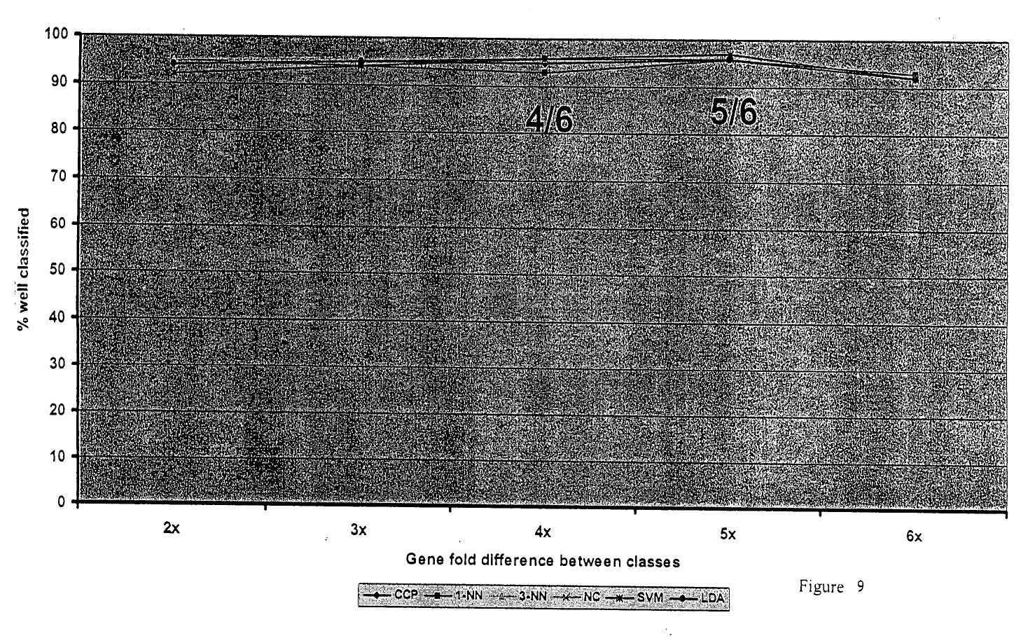

Cell samples were typically analyzed using classification algorithms to classify cell samples as belonging to either the ovarian cancer group or the normal ovarian group. The classification algorithms grouped the levels of expression for multiple marker genes for each cell sample, and then placed the cell sample into either the normal ovarian group or the ovarian cancer group. As shown in FIG. 9, the classification algorithms were able to properly group cell samples in approximately 92% to 98% of cell samples tested. In particular, cell samples were properly categorized using marker genes that were expressed at between two-fold and six-fold levels in cancer cell samples. Classification algorithms were most accurate when using HDGF, GAPDH, A-CRABP II, TPI, cytokeratin 18, cytokeratin 7, keratin 19, and α-enolase, all of which were expressed at five-fold higher levels in ovarian cancer cell samples (FIG. 9).

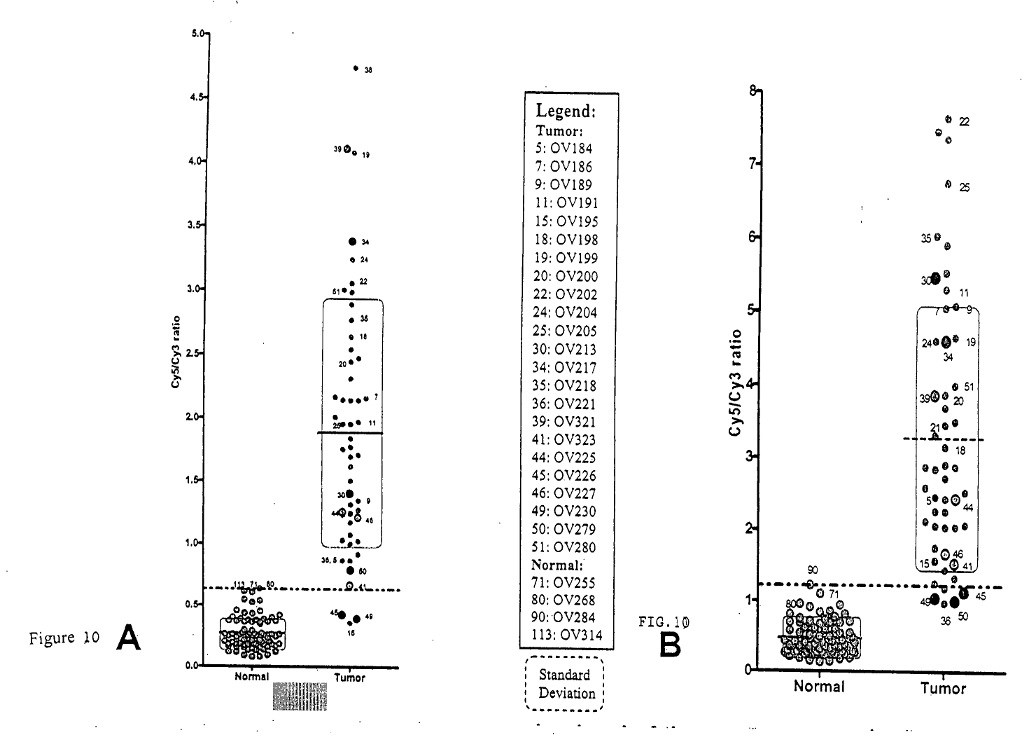

In addition to using multiple marker genes to diagnose cancer in a subject, individual marker genes were used to classify cell samples as either containing cancerous cells or containing solely normal cells. As shown in FIGS. 10A and 10B, α-enolase and TPI expression levels were determined and compared for ovarian cancer cell samples and normal ovarian cell samples. The ovarian cancer cell samples showed consistently higher levels of expression as compared to the normal ovarian cell samples (FIGS. 10A and 10B). The classification algorithms were used to classify cell samples, and classified the cell samples with greater than 90% accuracy.

It should be noted that these marker genes showed clear patterns of overexpression in ovarian cancer cell samples when compared to normal ovarian cells, which would allow for the proper classification of the cell sample without a classification algorithm. By comparing the level of expression of α-enolase and/or TPI in a cell sample to the level of expression for the same marker genes in a sample known to be neoplastic or non-neoplastic, a clinician would be able to identify whether the cell sample is neoplastic or non-neoplastic. A clinician would be able to review data obtained for a normal population or a population having ovarian cancer to determine the group that the cell sample most resembled. Therefore, there can be cases in which a classification algorithm is not necessary for determining the presence of cancer in a patient.

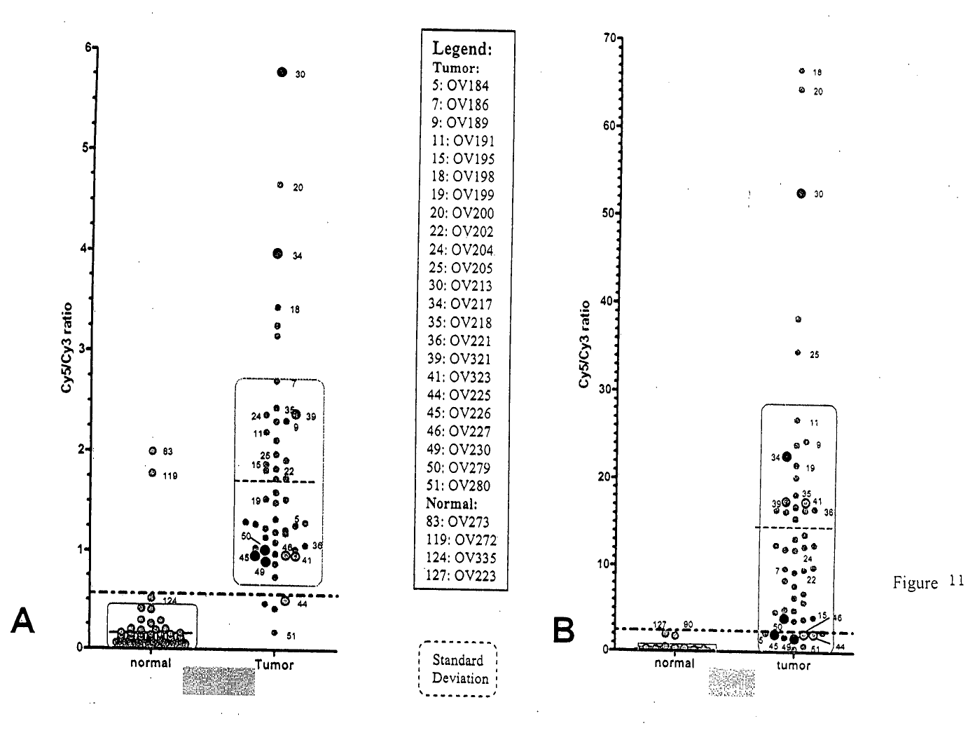

The level of expression of cytokeratin 18 was also tested to determine whether it could be used to distinguish between normal cell samples and cell samples containing cancer cells. The ovarian cancer cell samples showed an increased level of expression for cytokeratin 18 as compared to the normal ovarian cell samples in more than 90% of the cases analyzed (FIG. 11). Notably, two normal cell samples showed high levels of expression for cytokeratin 18 that were outside of the range for the other normal ovarian cell samples analyzed. As established with TPI and α-enolase, the level of expression of cytokeratin 18 in the cell sample was sufficiently increased in the ovarian cancer group for reliable classification of the cell samples using the classification algorithms and the training set. Also, cytokeratin 18 expression in cancer cell samples was sufficiently different from normal cell samples to allow proper identification of neoplastic cells using statistical techniques other than the classification programs described above.

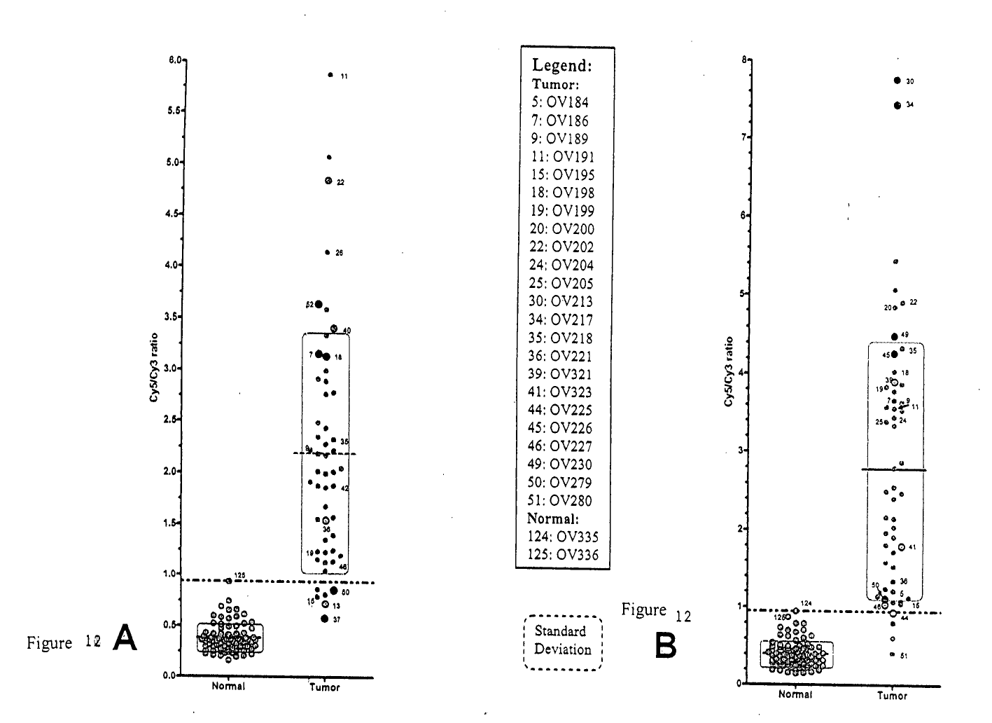

In addition to cytokeratin 18, HDGF and keratin 19 were consistently expressed at higher levels in almost all cancer cell samples tested as compared to normal ovarian cell samples (FIGS. 12A and 12B). Ovarian cancer cell samples showed a five-fold increase in the levels of expression for HDGF and keratin 19 over normal ovarian cell samples. Furthermore, HDGF and keratin 19 expression levels were increased in ovarian cancer cell samples when compared to normal ovarian cell samples in more than 90% and 83% of cell samples tested, respectively (FIGS. 12A and 12B). This indicates that these marker genes can be used individually to determine whether cancer exists in a cell sample.

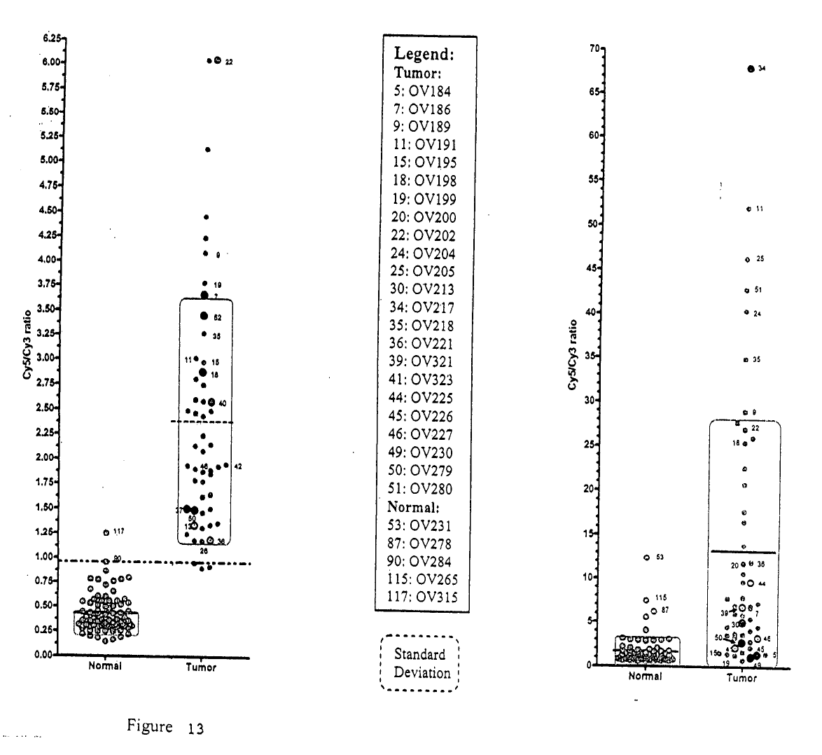

In FIG. 13, the level of expression for GAPDH is shown in normal ovarian subjects and in ovarian cancer patients. GAPDH is expressed at a five-fold higher level in ovarian cancer patients compared to normal ovarian subjects. In particular, GAPDH is expressed at higher levels in ovarian cancer cell samples in greater than 90% of patients tested (FIG. 13). These results show that GAPDH levels of expression can be determined by the focused microarray, and that the level of expression is consistently higher than the expression found in normal ovarian cell samples.

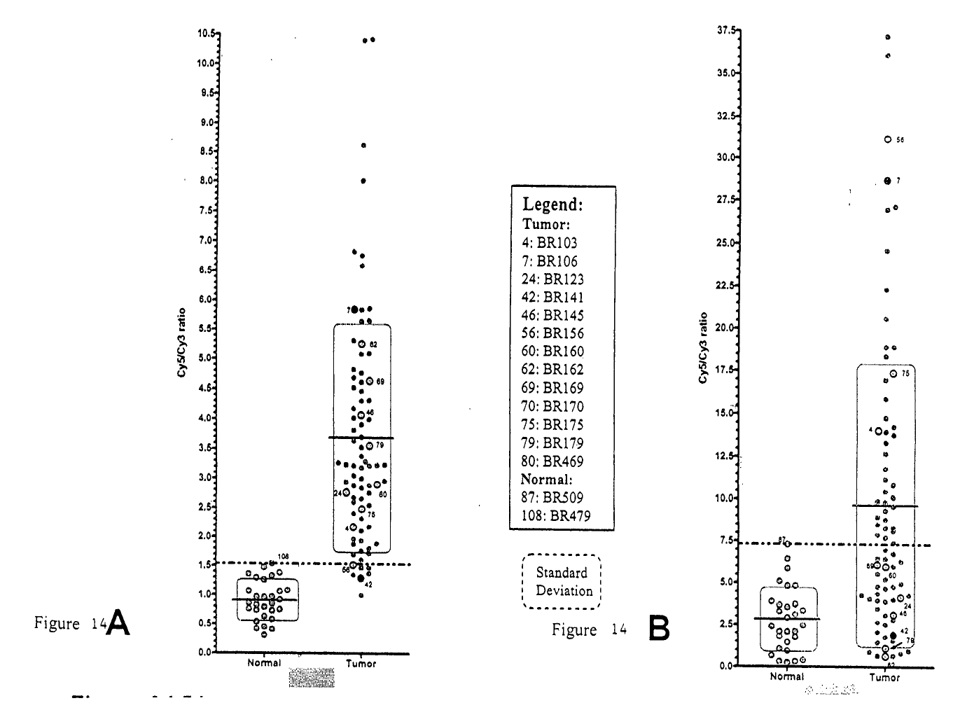

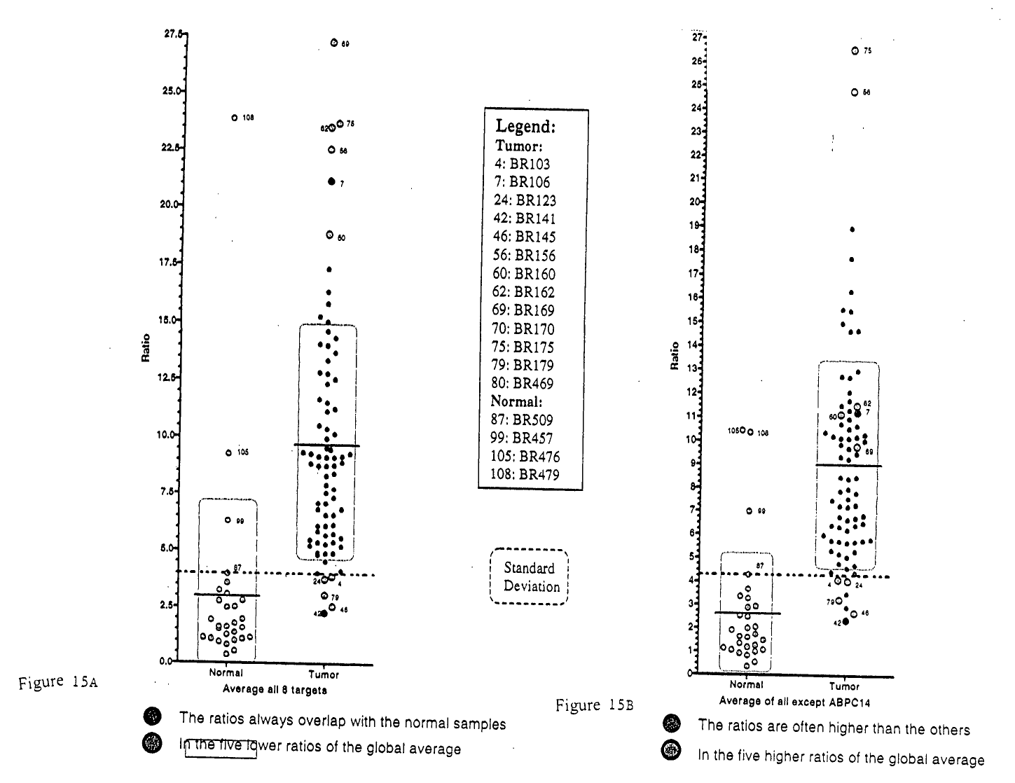

The microarray was further tested against breast cancer cell samples and normal breast cell samples using the same analytical techniques as for the ovarian tests. Cell samples were isolated from 103 individuals, and RNA was isolated as described in the Examples. As shown in FIGS. 14A and B, the marker genes cathepsin D showed three times higher levels of expression in breast cancer patients than normal breast subjects. Cathepsin D was particularly useful in classifying breast cell samples as either cancerous or normal (FIGS. 14A and 14B). Cathepsin D expression was increased in most cancer patients with the exception of eight patients that showed levels of expression for the marker gene at the upper range of normal breast subjects. By using this marker gene, breast cancer patients could be properly classified in greater than 90% of cases.

Moreover, the levels of expression for the marker genes cathepsin D, ezrin, keratin 19, s1c9a3r1, A-CRABP II, HER-2, estrogen receptor α, and FABP 4 were averaged for each breast cell sample isolated. The levels of expression of cathepsin D, ezrin, keratin 19, s1c9a3r1, A-CRABP II, HER-2, and estrogen receptor a are three fold higher in breast cancer cell samples as compared to normal breast cell samples (FIGS. 15A and 15B). Although there is overlap between the standard deviations of the breast cancer patients and the normal breast subjects, the majority of breast cancer cell samples showed expression levels that were higher than the majority of normal breast cell samples (FIG. 15A). The wide range for the standard deviation observed with the normal breast subjects can be attributed to two subjects that appear to be outliers from the rest of the normal breast group (FIG. 15A). When marker gene HER-2 was removed from the analysis, the overlap between the groups was further reduced, and most samples were more discernibly classified as either cancerous or normal (FIG. 15B). The classification algorithms were able to properly classify the cell samples as either neoplastic or non-neoplastic, even though there was overlapping standard deviations between marker genes.

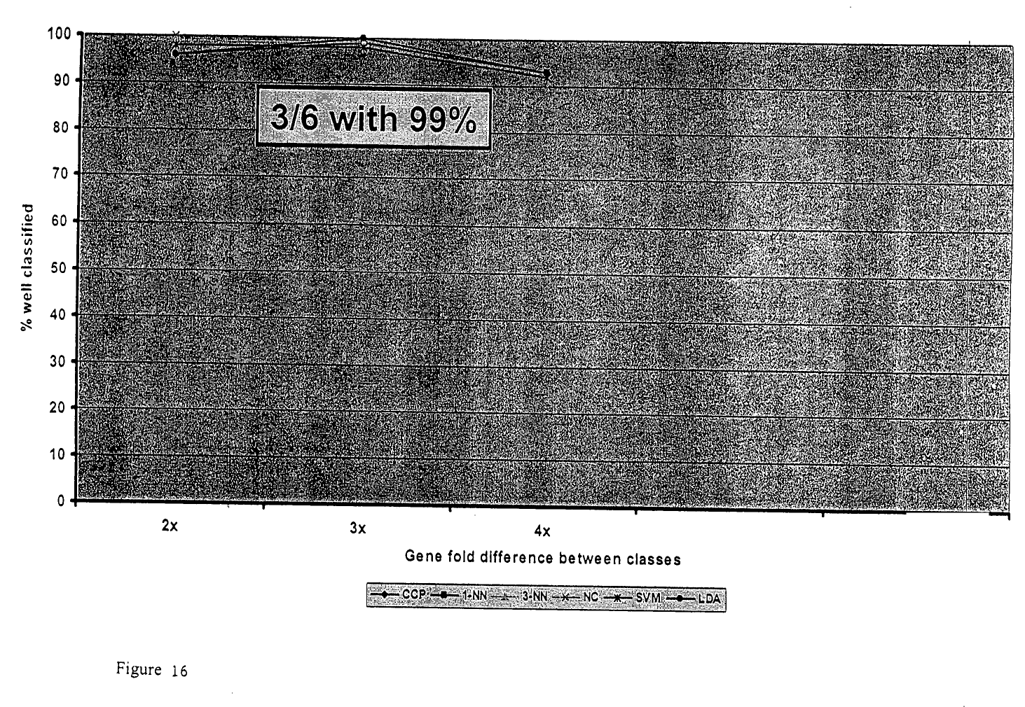

The overlap between the normal breast subjects and the breast cancer patients does not affect the proper classification of the cell samples by the classification algorithms. For the marker genes that are expressed at three fold higher levels in breast cancer patients, the classification algorithms were able to properly classify patients in 92% to 98% of cases (FIG. 16). Furthermore, marker genes expressed at two fold increased levels in breast cancer patients allowed for proper classification using the algorithms in 92% of the cases analyzed. Classification algorithms are therefore useful in cases in which the level of expression for the marker genes between normal cell samples and a diagnostic cell sample shows some similarity in expression.

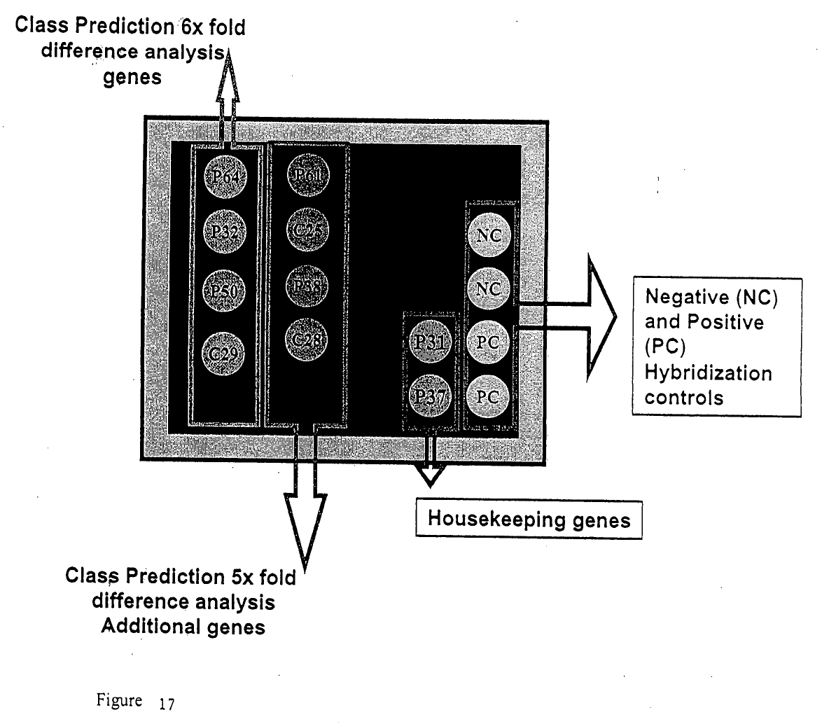

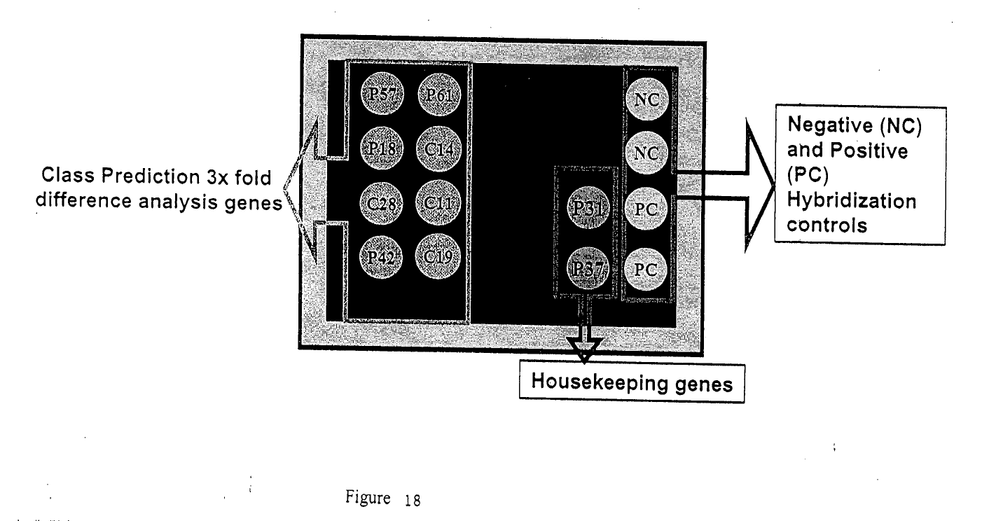

FIG. 17 shows a diagram of a focused microarray that was used for the ovarian classification studies. The microarray had the marker genes that were expressed at six-fold and five-fold higher levels in ovarian cancer cell samples. The chip also contained the marker genes EF2 and EIF-4B attached to its surface for normalization of the signal. Negative control (NC) and positive control (PC) capture probes were attached to the surface to ensure that non-specific hybridizations were not occurring on the chip. Additionally, a diagram of the focused microarray used for classification studies on breast cancer cell samples is shown in FIG. 18. Although this microarray is essentially the same as the ovarian focused microarray, the breast microarray contained marker genes that were expressed at three-fold higher levels in breast cancer cell samples. It should be recognized by those of skill in the art that these embodiments are merely illustrative examples, and should not be construed as limiting demonstrations of the type of focused microarrays that can be used to diagnose cancer in a subject.

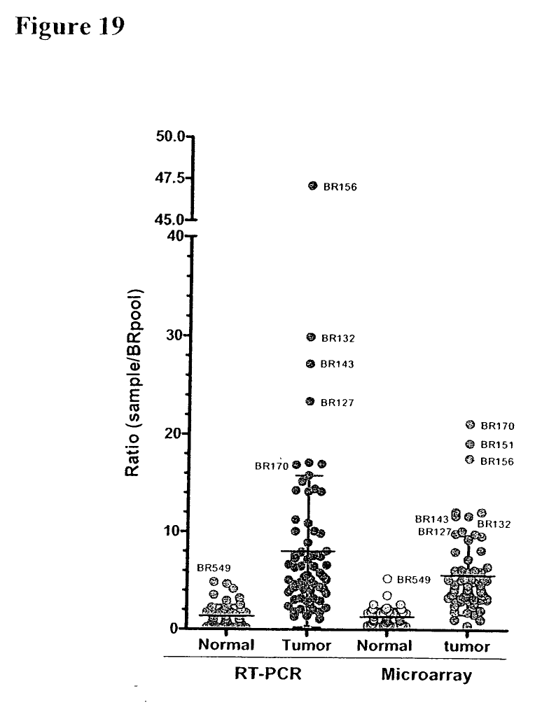

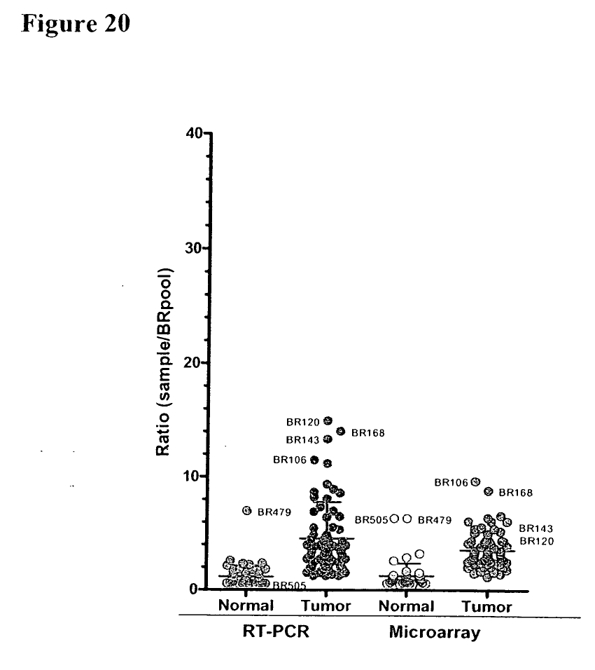

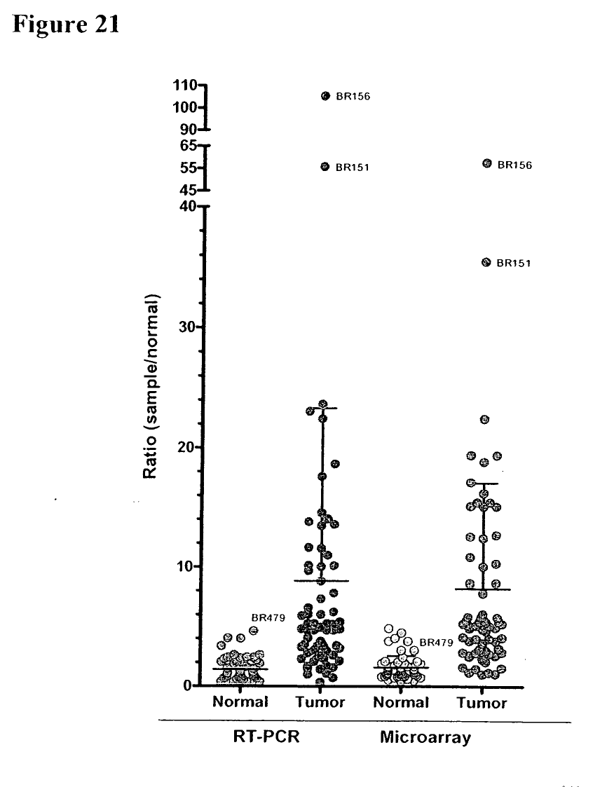

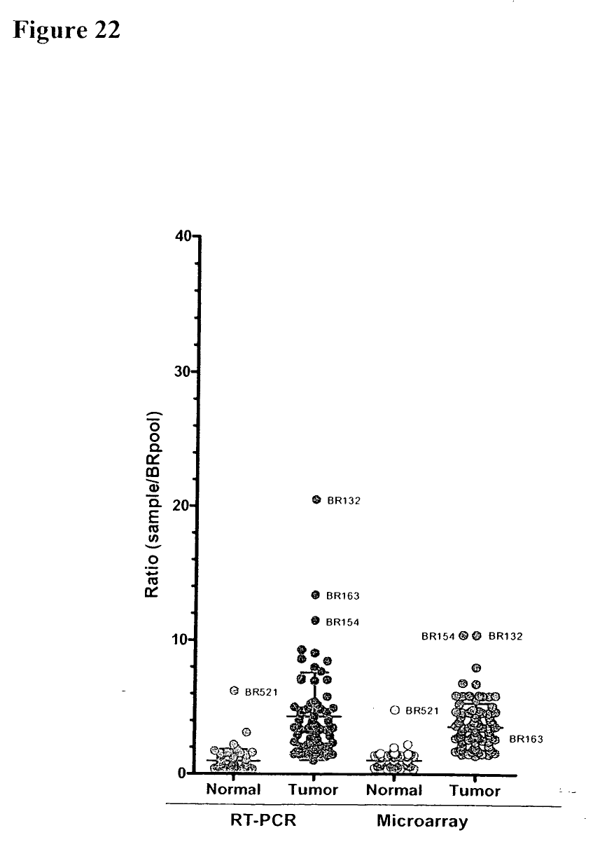

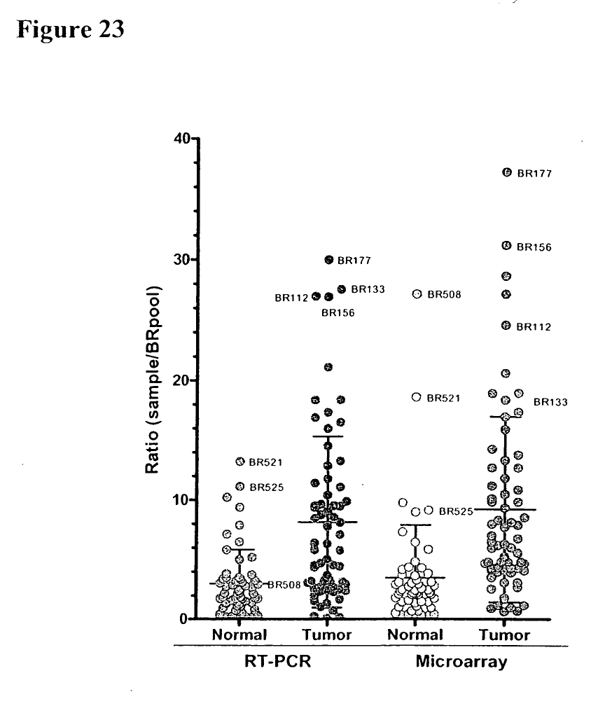

Quantitative RT-PCR confirmed the results obtained using the focused microarray for five marker genes (FIGS. 19-25). The marker genes were keratin 19, ezrin, s1c9a3r1, cathepsin D, and ACRAB-PII. In all cases, quantitative RT-PCR confirmed the results obtained by microarray. As can be seen for keratin 19 and ezrin, the ratios for normal samples versus the BRPool were similar for essentially all normal samples (FIGS. 19 and 20). Ratios were produced by comparing the level of expression of the marker genes in normal or tumor samples to the level of expression of the marker genes to normal breast samples pooled into a group, which was named the BRPool. This was also true for individual patients, as indicated by the number designations (FIGS. 19 and 20). The similarities between quantitative RT-PCR findings and the microarray were seen for ratios of expression between tumor samples and the BRPool (FIGS. 19 and 20). Such similarities can be seen for the other marker genes as well (FIGS. 23-25).

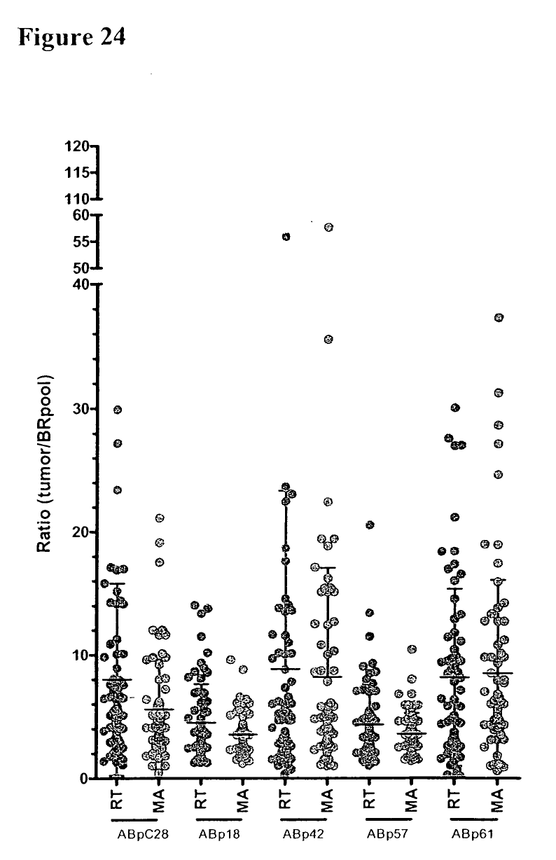

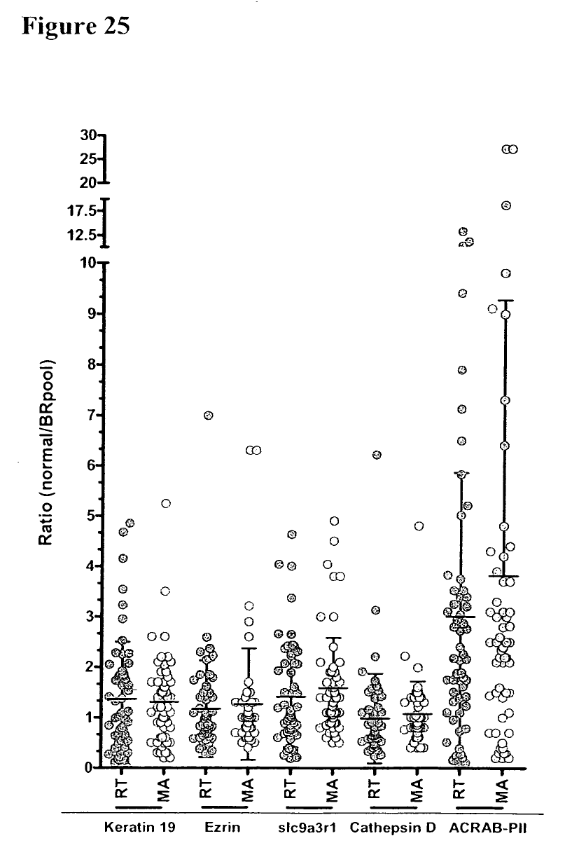

In addition, the results shown above for tumor samples versus the BRPool for all five marker genes are directly compared in FIG. 24. Quantitative RT-PCR and the microarray analysis showed similar ratios of expression for the five marker genes between tumor samples and the normal breast pool (BrPool) (FIG. 24). A similar comparison is shown in FIG. 25 where the quantitative RT-PCR analysis and the focused microarray analysis yielded similar results for the five marker genes in normal samples as compared to the BRPool.

Table 7 summarizes results from a comparison of the focused microarray and the quantitative RT-PCR analysis.

TABLE 7

| | | | | | | | Contrary |
|---|---|---|---|---|---|---|---|
| Targets | Comparable | | >2× Lower | | >2× Higher | | Results |
| Keratin 19 | 99 | (80.5) | 8 | (6.5) | 15 | (12.2) | 1 (0.8) |
| Ezrin | 96 | (78) | 12 | (9.8) | 15 | (12.2) | 0 |
| Slc9a3r1 | 105 | (85.4) | 15 | (12.2) | 3 | (2.4) | 0 |
| Cathepsin D | 112 | (91.1) | 8 | (6.5) | 3 | (2.4) | 0 |
| ACRAB-PII | 99 | (80.5) | 17 | (3.8) | 5 | (4.0) | 2 (1.6) |

In most cases, quantitative RT-PCR and the focused microarray yielded comparable results in which both techniques produced results that allowed for the proper classification of the tissue sample. It was rarely the case that the ratio between a sample and the BRPool was lower than 0.5 with one screening method and higher than 2 with the other method. In general, there was a correspondence between results in at least 78% of tissues tested.

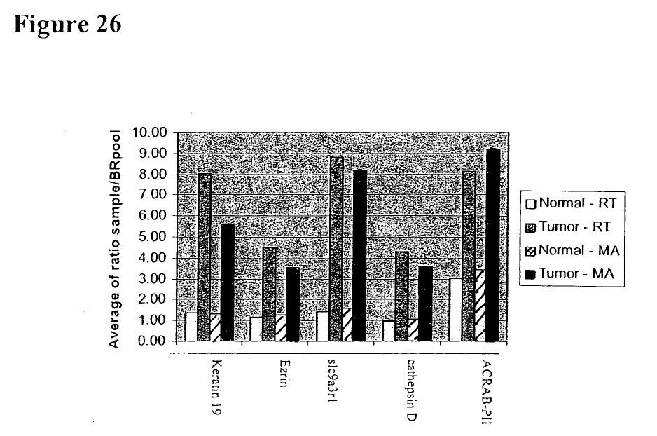

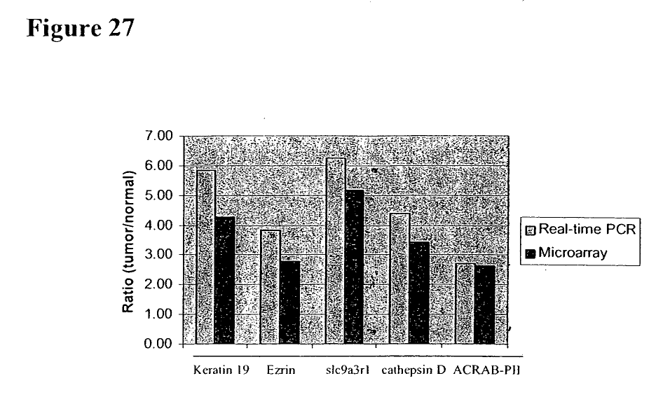

FIG. 26 shows the average ratios of expression found in tumor samples and normal samples as compared to the BRPool using the focused microarray (MA) and quantitative RT-PCR (RT). The findings were not dependent on the technique used, but were dependent on the marker gene. Similar results are shown in FIG. 27.

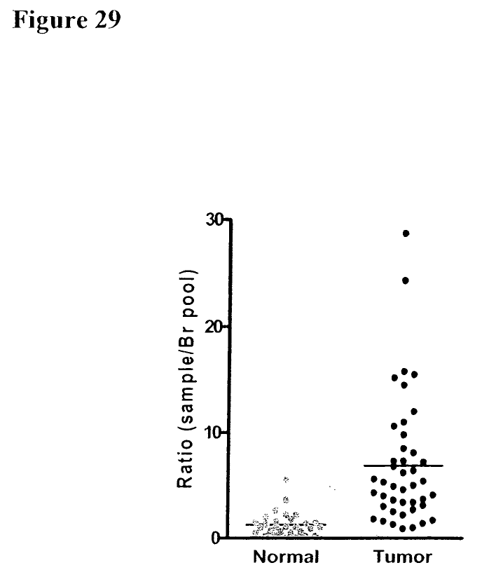

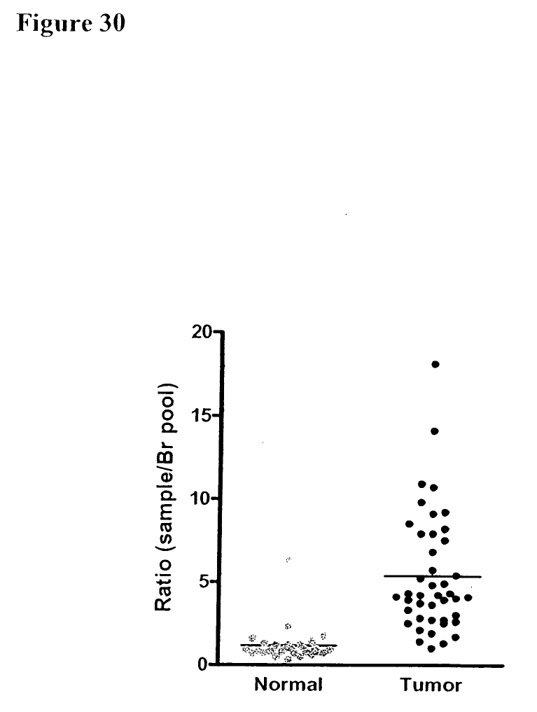

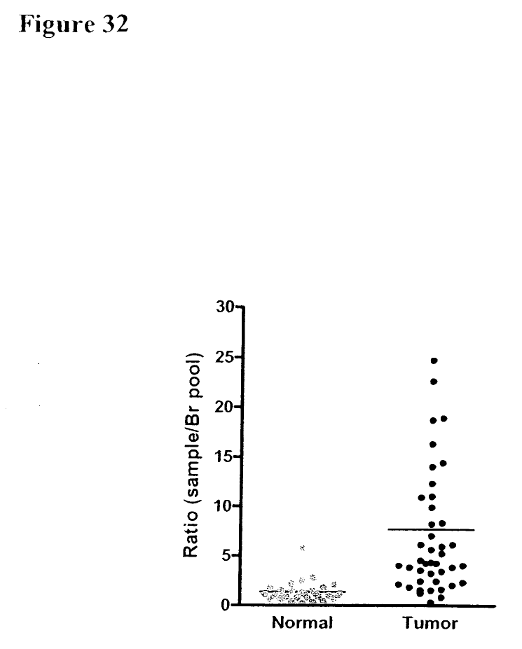

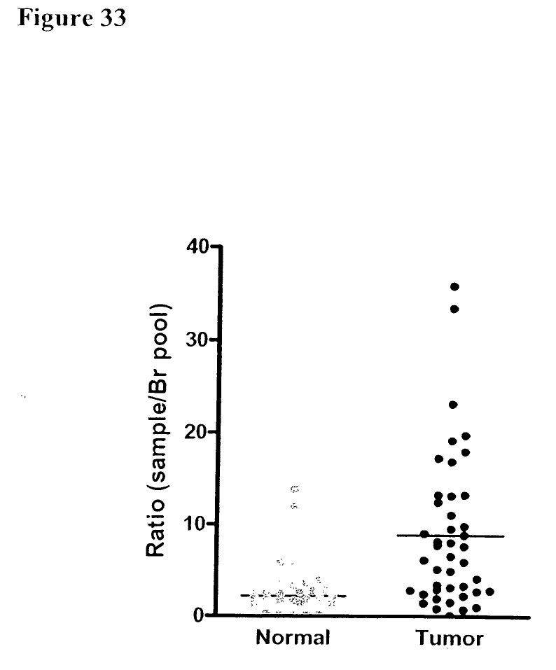

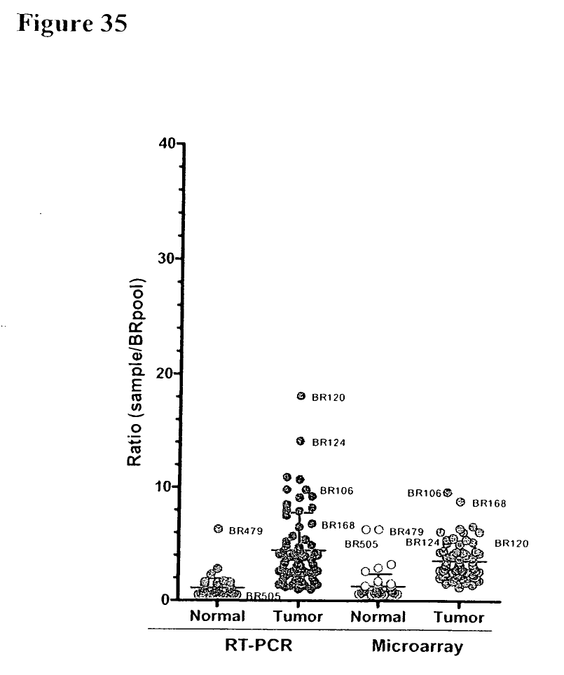

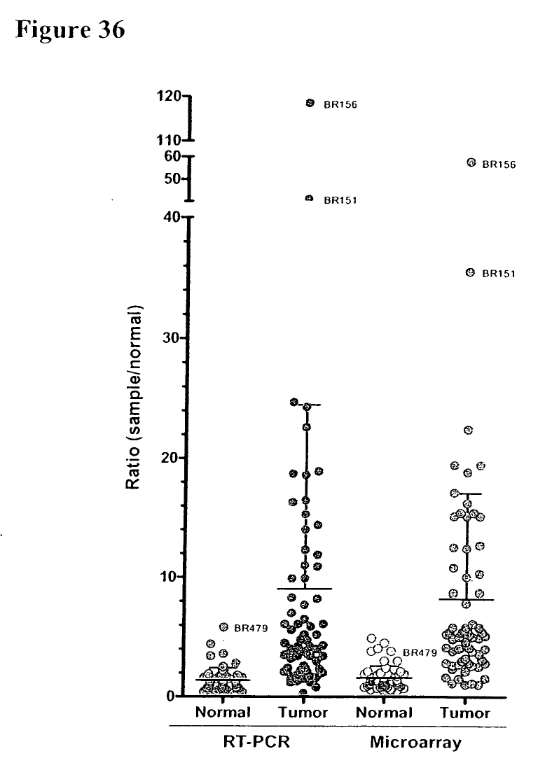

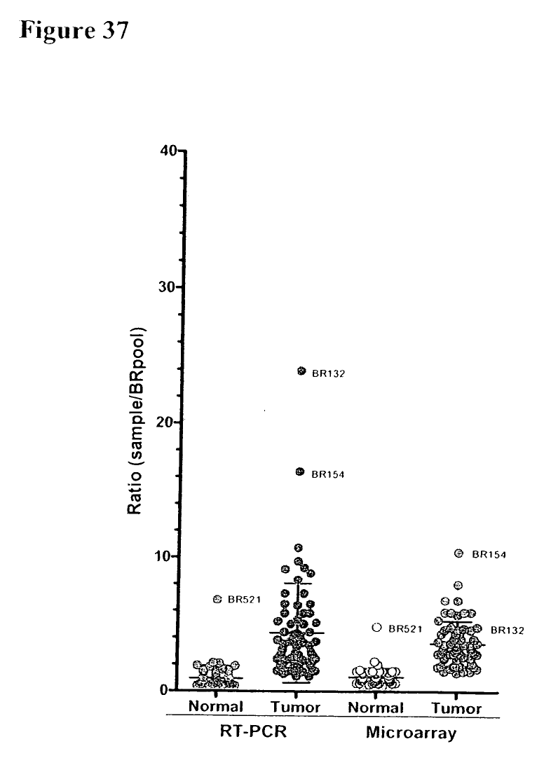

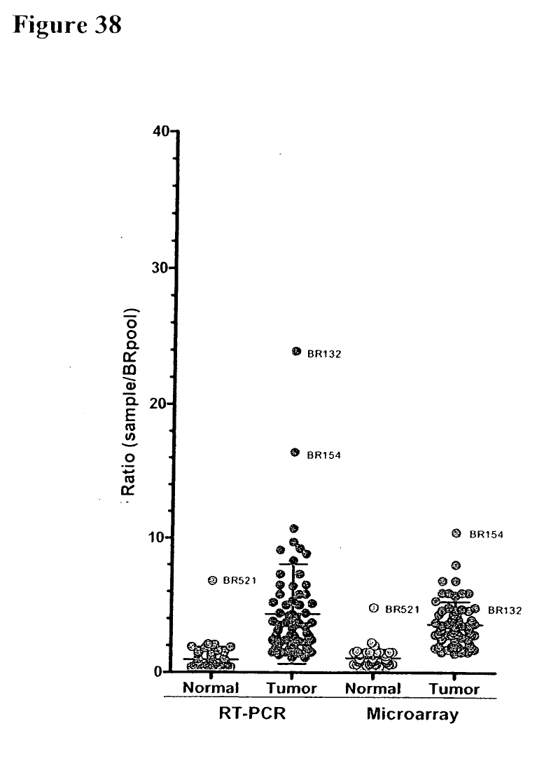

FIGS. 29-38 show the ratios of expression of marker genes keratin 19, s1c9a3r1, ezrin, cathepsin D, and ACRAB-PII, as determined by quantitative RT-PCR, for normal breast samples and tumor breast samples. Ratios were produced by comparing the level of expression of the marker genes in normal or tumor samples to the level of expression of the marker genes to normal breast samples pooled into a group, which was named the BRPool. FIGS. 29-33 show that tumor samples had much higher ratios as compared to normal breast samples. Furthermore, keratin 19, s1c9a3r1, and ACRAB-PII had the highest ratios of the marker genes (FIGS. 29, 32, and 33). The results are summarized in Table 8.

TABLE 8

| | Fold Increase in | Fold Increase in |
|---|---|---|
| Targets | Tumor Ratio (RT-PCR) | Tumor Ratio (microarray) |
| Keratin 19 | 5.4 | 4.5 |
| Ezrin | 4.6 | 2.5 |
| Slc9a3r1 | 5.7 | 5.0 |
| Cathepsin D | 6.4 | 3.8 |
| ACRAB-PII | 2.9 | 2.0 |

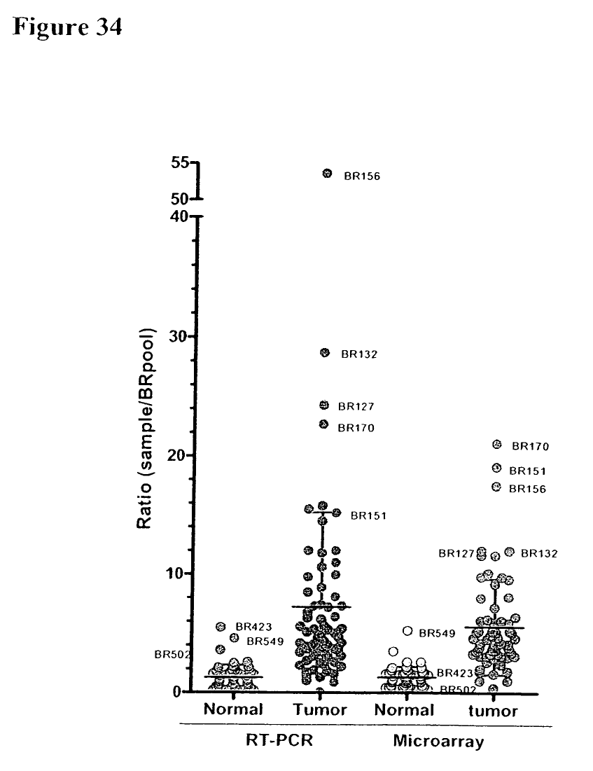

These results were compared to the results obtained for these marker genes using the focused microarray (FIGS. 34-38). The focused microaray and quantitative RT-PCR analyses yielded similar results for all marker genes (FIGS. 34-38). In particular, the results obtained using the focused microarray and quantitative RT-PCR were nearly indistinguishable for keratin 19, s1c9a3r1, cathepsin D, and ACRAB-PII (FIGS. 34 and 36-38). Therefore, the focused microarray results were confirmed by RT-PCR analysis.

### EXAMPLES

Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Classification of Cell Samples Isolated from Ovarian Cancer Patients and Normal Ovarian Sublects

1. Patient Samples and Normal Samples

Patient material was purchased from Asterand, Inc. (Detroit, Mich.), Clinomics Biosciences, Inc (Watervliet, N.Y.) and Biochain Institute, Inc. (Hayward, Calif.). For the ovarian cancer groups, only patients with greater than 70% tumor cell content in the tumor mass were included in the studies. Total RNA was extracted from snap frozen tissue samples with the Trizol Reagent kit (Gibco-BRL, Carlsbad, Calif.) using the recommended extraction procedures of Asterand, Inc., Clinomics Biosciences, Inc. or Biochain Institute, Inc. Total RNA was treated with RNA-free DNAse I (New England BioLabs, Beverly, Mass.) and purified with the RNEasy kit (Qiagen, Hilden, Germany). RNA samples were visualized on an Agilent 2100 BioAnalyzer (Agilent Technologies, Foster City, Calif.).

Each patient included in the study was screened against the same normal total RNA pool in order to compare them together. Ovarian normal total RNA pools were purchased from Biochain Institute, Inc. (Hayward, Calif.). Tables 1 and 2 show the clinical characteristics of the patients included in normal pools. The ovarian normal pool was composed of 20 cases. The normal ovarian subjects (79 cases) and the ovarian cancer patients (57 cases) were screened on the focused microarray in triplicate. A majority of serous adenocarcinoma patients were included in the study. Stage 3 represented the most common carcinoma stage included in this study, but it should be noted that all stages were represented. The majority of the women (40 cases) were lymph node negative.

2. Total RNA Controls Utilized in the Present Study

Total RNA received from the three suppliers described above was quantified with the NanoDrop® ND-1000 spectrophotometer (NanoDrop Technologies, Wilmington, Del.), and the ratio A260/A280 was calculated. Total RNA with an A₂₆₀/A₂₈₀ ratio between 1.9-2.3 in 10mM Tris, pH 7.5 was included in the study. Integrity of the ribosomal RNA was visualized by standard 1% agarose gel electrophoresis in TBE buffer (Tris 9 mM; 9 mM borate acid; 0.2 mM EDTA) containing 0.04% EtBr. RNA samples without apparent RNA degradation, which was indicated by appropriate rRNA 28S/18S ratios, were used for the first strand cDNA labeling reaction.

3. Preparation of Positive Control RNA From Arabidopsis thaliana Chlorophyll Synthase G4

A plasmid containing the Arabidopsis thaliana chlorophyll synthase G4 gene was obtained from the BRI Institute (Clear Water Bay, Kowloon Hong Kong, PRC). Briefly, five μg of a Midi preparation of the plasmid was prepared by the HiSpeed™ Plasmid Midi kit using the manufacturer's recommended procedures (Qiagen). Purified plasmid was digested with 40 U Sac I enzyme (New England BioLabs, Beverly, Mass.) for 2 hrs at 37° C., and the fragment was purified with the QIAquick PCR purification kit (Qiagen). The quality and identity of the fragment was verified by 1 % agarose electrophoresis in TBE buffer (Tris 9 mM; 9 mM borate acid; 0.2 mM EDTA) containing 0.04% EtBr.

In vitro transcription of 2 μg Sac I digestion was performed in 10× transcription buffer (400 mM Tris-HCl, pH 8.0; 60 mM MgC12; 100 mM DTT; 20 mM Spermidin) containing 2 μl of 10 mM NTP mix (Invitrogen Corp., Carlsbad, Calif.), 20 U RNAse OUT (Invitrogen Corp., Carlsbad, Calif.) and 50 U T7 RNA polymerase (New England BioLabs, Beverly, Mass.) for 2 to 30 hrs at 37° C. The reaction was then treated with 2 U DNAse I (Invitrogen Corp., Carlsbad, Calif.) in 10× DNAse buffer (200 mM Tris-HCl, pH 8.4; 20 mM MgC12; 500 mM KCl) for 15 min. at 37° C. The RNA was cleaned with the RNEasy kit (Qiagen) and quantified by an Ultrospec 2000 spectrophotometer (Pharmacia Biotech, Piscataway, N.J.). The positive control was aliquoted and conserved at −80° C.

4. First Strand cDNA Labeling of RNA

To obtain probes for hybridization to the focused microarray, first cDNA labeling reactions were performed on RNA pools and RNA isolated from ovarian cancer patients and normal ovarian subjects. The labeling reaction was done from 10 μg total RNA of the patient to be tested and same amount of starting material (10 μg) was done separately for the normal pool corresponding. In brief, total RNA was incubated with 2 ng control positive RNA of Arabidopsis thaliana, 3 μg Oligo (dT) 12-18 primer (Invitrogen Corp., Carlsbad, Calif.), 1 μg PdN6 random primer (Amersham, PLC, Buckinghamshire, UK) for 10 min. at 65° C., put on ice for 2 min. then diluted in the labeling reaction buffer, 5× First strand buffer (50 mM Tris-HCl, pH 8.3; 75 mM KCl; 3 mM MgCl₂); 20 mM DTT; 0.5 mM dATP; 0.5 mM dTTP; 0.5 mM dGTP; 0.05 mM dCTP; 26 μM Cy5-dCTP or 52 μM Cy3-dCTP) (Perkin Elmer, Wellesley, Mass.) and 400 U SuperScript III RNAse H— RT (Invitrogen Corp., Carlsbad, Calif.). After incubation of 5 min. at 25° C., the reaction was put at 42° C. for 90 min. then 400 U of SuperScript II RNAse H— RT was added and the reaction was continued for another 90 min.

Digestion of the labeled cDNA with 5 U RNAse H (New England BioLabs, Beverly, Mass.) and 40 U RNAse A (Amersham PLC, Buckinghamshire, UK) was done at 37° C. for 30 min. The labeling probe was purified with the QIAquick PCR purification kit (Qiagen, Hilden, Germany) protocol with some modifications. In brief, the reaction volume was completed to 50 μl with DEPC H₂O and 2.7 μl 2 M NaOAc, pH 5.2 was added. The reaction was diluted with 200 μl PB buffer, put on the purification column, spun 15 sec. at 10,000×g, followed by 3 washes of 500 μl PE buffer (15 sec. at 10,000×g) and eluted two times in 50 μl DEPC H₂O total (1 min. at 10,000×g). Frequency of incorporation and amount of cDNA labeled produced were evaluated for both labeled dCTPs by spectrophotometer (NanoDrop® ND-1000, USA) at 260 nm, 550 nm and 650 nm. The labeled material was dry by speed vacuum (Savant SC110A, USA) and resuspended in 3.75 μl H₂O total for both the Cy5-dCTP labeled samples and the Cy3-dCTP labeled samples. Finally, a dye swap reaction was performed for each patient on the same day to account for the potential differential incorporation of the Cye-dCTP dyes used in the first strand cDNA labeling reaction.

5. Focused Microarray Preparation

Oligonucleotides 68 nucleotides in length corresponding to targets-of-interest were designed by analyzing sequences showing less than 30% identity base to base with other coding sequences (cds) submitted to NCBI bank. The comparison was done using the Blast research program (Altschul et al. (1990) J. Mol. Biol. 215: 403-410). For the focused microarray, the basic melting point temperature (Tm; salt concentration of 50 mM Na+) of each oligonucleotide was calculated, and averaged 76.69° C.±3.92° C. The total GC content averaged 51.47%±9.75%. Two negative controls (68 bp of antisense cds of the ABpC1 and ABp6 targets) were also synthesized (ABpC1N and ABp6N). Oligonucleotides selection on the focused microarray was based on microarray results obtained on two previous versions of the microarray, and on comparison to 2D proteomic analysis. The complete group of oligonucleotides is shown above in the Detailed Description.

The oligonucleotides were synthesized by Integrated DNA Technologies (IDT, Coralville, Iowa). Quality control was done by MALDI-TOF Mass spectrometry (IDT, Coralville, Iowa). Moreover, an Electrospray MS analysis was done in order to confirm an oligonucleotide's position on the plate used for printing, and represented a second independent verification of oligonucleotide integrity. Electrospray MS analysis can be performed by commercial service providers such as West Coast Analytical Services (Santa Fe Springs, Calif.).

Different dilutions of positive control DNA from Arabidopsis thaliana Chlorophyll Synthetase G4 (from undiluted 200 ng/μl: 1:2, 1:4, 1:8, 1:16, 1:32, 1:64) were printed for each grid and used for the normalization process. Oligonucleotide concentration was normalized to 40 uM in 50% DMSO and printed on UltraGAPS™ (Corning, Corning, N.Y.) by the VersArray CHIP Writer Prosystems (BioRad Laboratories, Hercules, Calif.) with the Stealth Micro Spotting Pins (#SMP3) (Telechem International, Inc., Sunnyvale, Calif.). Each oligonucleotide was printed in triplicate on a grid. Each focused microarray contained three grids. Buffer (50% DMSO) and Salmon Testis DNA (0.2 μg/ml in 50% DMSO; Sigma D-7656, Sigma Corp, St. Louis, Mo.) were also printed for BioChip analysis step. Blank spots were also done and used for analysis steps. After the printing, the slides were dry by overnight incubation in the CHIP Writer chamber, treated by UV Stratalinker (Stratagene, La Jolla, Calif.) at 600 mJoules and baked in an oven for at least 6 to 8 hours.

Printing quality control was done with the PARAGON™ DNA Microarray Quality Control Stain kit (Molecular Probes, Invitrogen Corp., Carlsbad, Calif.) following the manufacturer's recommendations. The slides were scanned with the ScanArray™ Lite MicroArray Scanner (Perkin Elmer, Wellesley, Mass.). The quality control test was done on at least one slide per printing run.

6. Hybridization Conditions

The focused microarray slide was pre-washed before the prehybridization step. First, the microarray was preheated at 42° C. for 20 min in 2×SSC (300 mM NaCl; 30 mM sodium citrate)/0.2% SDS under agitation. The slide was subsequently incubated for 5 min. at room temperature in 0.2×SSC (30 mM NaCl, 3 mM sodium citrate) under agitation and then agitated for 5 min. at room temperature in DEPC H₂O. The slide was spun dry at 1000×g for 5 min. and prehybridized in DIG Easy Hyb Buffer (Roche Diagnostics Corp., Indianapolis, Ind.) containing 0.02% Bovine Serum Albumin (Roche, USA) at 42° C. in humid chamber for 3 hrs then washed two times in DEPC H2O, and once in Isopropanol (Sigma, USA, St. Louis, Mo.) and spun dry at 1000×g for 5 min.

To the mixed Cy3/Cy5 probe prepared as described above, 15 μg Baker tRNA (Roche Diagnostics Corp., Indianapolis, Ind.) and 1 μg Cot-1 DNA (Roche Diagnostics Corp., Indianapolis, Ind.) were added. The probe was incubated 5 min. at 95° C., put on ice for 1 min. and diluted with 14 μl DIG Easy Hyb buffer (Roche Diagnostics Corp., Indianapolis, Ind.). The probe was spun for two min. at 100×g and incubated at 42° C. for at least 5 min.

The three supergrids on the slide were separated by a Jet-Set Quick Dry TOP Coat 101 line (L'OREAL, Paris #FX268). Each probe was added to its respective supergrid and covered by a preheated (42° C.) coverslip (Mandel Scientific Company Ltd., Guelph, ON, CA). The slide was incubated at 42° C. in humid chamber for at least 15 hrs.

The coverslips were removed by dipping in 1×SSC (150 mM NaCl; 15 mM sodium citrate)/0.2% SDS solution preheated at 50° C. Slides were washed 3 times for 5 min./wash in 1×SSC (150 mM NaCl; 15 mM sodium citrate)/0.2% SDS solution preheated at 50° C. with agitation, 3 times in 0.1×SSC (15 mM NaCl; 1.5 mM sodium citrate)/0.2% SDS solution preheated at 37° C. with agitation and once in 0.1×SSC (15 mM NaCl; 1.5 mM sodium citrate) with agitation for 5 min. The slides were dipped several times in DEPC H2O. Slides were then spun dry at 1000×g for 5 min.

7. Statistical Analysis

Focused microarray slides were scanned with the ScanArray™ Lite MicroArray Scanner (Packard BioSciences, Perkin Elmer, Wellesley, Mass.) and the analysis was done with the QuantArray® Microarray Analysis software version 3.0 (Packard BioSciences, Perkin Elmer, Wellesley, Mass.) using the adaptative method.

The QuantArray® data results were analyzed by the spot background subtracted values for Cy5 and Cy3 channels. Spots with signal to noise ratios lower than 1.5 were discarded. The signal intensity was adjusted to a minimum of 100 and spots with signal value lower than 100 in both channels were discarded. Normalization of the ratios with the spike positive control (Arabidopsis thaliana) was done normalize the ratio to one for that control on each slide. The slides were discarded when the negative and/or positive controls failed, i.e., those with high background. The average of the ratios for each target was done between the direct and the reciprocal labeling reactions. Statistical analysis was done with the ArrayStat 1.0 (Imaging Research Inc., St. Catharines, Ontario, Calif.). A log transformation of the ratio data was followed by a Student T test for two independent conditions using a proportional model without offsets at a p<0.05 thresholds. A significant increase (ratio Cy5/Cy3 higher than 2.0) or decrease (ratio Cy5/Cy3 lower than 0.5) are considered to be significant if the p value is lower than 0.05.

8. Classification Algorithms

Class prediction analyses were performed using the BRB ArrayTools developed by Dr. Richard Simon (NIH/NCI) and Amy Peng. Briefly, class prediction analyses were done on the results obtained for each patient in the study. Patients were divided into two classes following their malignancy: normal class and tumor class. These classes became the training sets by which patients were compared for purposes of classification. The classification algorithms used the expression data from the training sets to make all patient classifications during the tests. Class determination was done based on the clinical data associated to each patient. FIGS. 10A, 10B, and 11-13 show the results of several class prediction analyses on individual marker genes. There were six different classification algorithms used in the studies: compound covariate predictor, diagonal linear discriminant analysis, nearest neighbor predictor (1-NN and 3-NN), nearest centroid predictor and support vector machine predictor. Those analyses permitted the development of a multi-gene classifier to predict the class for a new sample and estimate the misclassification rates. Cross-validation of the class prediction classifiers were done by the leave one-out study and permutation tests (n=2000) were conducted to address significance of the cross-validation test error rate.

### Example 2

### Classification of Cell Samples Isolated From Breast Cancer Patients and Normal Breast Subiects

1. Patient Samples and Normal Samples

Patient material was purchased from Asterand, Inc. (Detroit, Mich.), Clinomics Biosciences, Inc (Watervliet, N.Y.) and Biochain Institute, Inc. (Hayward, Calif.). For the breast cancer groups, only patients with greater than 70% tumor cell content in the tumor mass were included in the studies. Total RNA was extracted from snap frozen tissue samples with the Trizol Reagent kit (Gibco-BRL, Carlsbad, Calif.) using the recommended extraction procedures of Asterand, Inc., Clinomics Biosciences, Inc. or Biochain Institute, Inc. Total RNA was treated with RNA-free DNAse I (New England BioLabs, Beverly, Mass.) and purified with the RNEasy kit (Qiagen, Hilden, Germany). RNA samples were visualized on an Agilent 2100 BioAnalyzer (Agilent Technologies, Foster City, Calif.).

Each patient included in the study was screened against the same normal total RNA pool in order to compare them together. Breast normal total RNA pools were purchased from Biochain Institute, Inc. (Hayward, Calif.). Tables 1 and 2 show the clinical characteristics of the patients included in normal pools. The breast normal pool was composed of 20 cases. The normal breast subjects (79 cases) and the breast cancer patients (57 cases) were screened on the focused microarray in triplicate.

2. Total RNA Controls Utilized in the Present Study

Total RNA received from the three suppliers described above was quantified with the NanoDrop® ND-1000 spectrophotometer (NanoDrop Technologies, Wilmington, Del.), and the ratio A₂₆₀/A₂₈₀ was calculated. Total RNA with an A₂₆₀/A₂₈₀ ratio between 1.9-2.3 in 10 mM Tris, pH 7.5 was included in the study. Integrity of the ribosomal RNA was visualized by standard 1% agarose gel electrophoresis in TBE buffer (Tris 9 mM; 9 mM borate acid; 0.2 mM EDTA) containing 0.04% EtBr. RNA samples without apparent RNA degradation, which was indicated by appropriate rRNA 28S/18S ratios, were used for the first strand cDNA labeling reaction.

3. Preparation of Positive Control RNA From Arabidopsis thaliana Chlorophyll Synthase G4

A plasmid containing the Arabidopsis thaliana chlorophyll synthase G4 gene was obtained from the BRI Institute (Clear Water Bay, Kowloon Hong Kong, PRC). Briefly, five μg of a Midi preparation of the plasmid was prepared by the HiSpeed™ Plasmid Midi kit using the manufacturer's recommended procedures (Qiagen). Purified plasmid was digested with 40 U Sac I enzyme (New England BioLabs, Beverly, Mass.) for 2 hrs at 37° C., and the fragment was purified with the QIAquick PCR purification kit (Qiagen). The quality and identity of the fragment was verified by 1% agarose electrophoresis in TBE buffer (Tris 9 mM; 9 mM borate acid; 0.2 mM EDTA) containing 0.04% EtBr.

In vitro transcription of 2 μg Sac I digestion was performed in 10× transcription buffer (400 mM Tris-HCl, pH 8.0; 60 mM MgCl₂; 100 mM DTT; 20 mM Spermidin) containing 2 μl of 10 mM NTP mix (Invitrogen Corp., Carlsbad, Calif.), 20 U RNAse OUT (Invitrogen Corp., Carlsbad, Calif.) and 50 U T7 RNA polymerase (New England BioLabs, Beverly, Mass.) for 2 to 30 hrs at 37° C. The reaction was then treated with 2 U DNAse I (Invitrogen Corp., Carlsbad, Calif.) in 10× DNAse buffer (200 mM Tris-HCl, pH 8.4; 20 mM MgC12; 500 mM KCl) for 15 min. at 37° C. The RNA was cleaned with the RNEasy kit (Qiagen) and quantified by an Ultrospec 2000 spectrophotometer (Pharmacia Biotech, Piscataway, N.J.). The positive control was aliquoted and conserved at −80° C.

4. First Strand cDNA Labeling of RNA

To obtain probes for hybridization to the focused microarray, first cDNA labeling reactions were performed on RNA pools and RNA isolated from breast cancer patients and normal breast subjects. The labeling reaction was done from 10 μg total RNA of the patient to be tested and same amount of starting material (10 μg) was done separately for the corresponding normal pool. In brief, total RNA was incubated with 2 ng control positive RNA of Arabidopsis thaliana, 3 μg Oligo (dT) 12-18 primer (Invitrogen Corp., Carlsbad, Calif.), 1 μg PdN6 random primer (Amersham, PLC, Buckinghamshire, UK) for 10 min. at 65° C., put on ice for 2 min. then diluted in the labeling reaction buffer, 5× First strand buffer (50 mM Tris-HCl, pH 8.3; 75 mM KCl; 3 mM MgCl₂); 20 mM DTT; 0.5 mM dATP; 0.5 mM dTTP; 0.5 mM dGTP; 0.05 mM dCTP; 26 μM Cy5-dCTP or 52 μM Cy3-dCTP) (Perkin Elmer, Wellesley, Mass.) and 400 U SuperScript III RNAse H— RT (Invitrogen Corp., Carlsbad, Calif.). After incubation of 5 min. at 25° C., the reaction was put at 42° C. for 90 min. then 400 U of SuperScript II RNAse H— RT was added and the reaction was continued for another 90 min.

Digestion of the labeled cDNA with 5 U RNAse H (New England BioLabs, Beverly, Mass.) and 40 U RNAse A (Amersham PLC, Buckinghamshire, UK) was done at 37° C. for 30 min. The labeling probe was purified with the QIAquick PCR purification kit (Qiagen, Hilden, Germany) protocol with some modifications. In brief, the reaction volume was completed to 50 μl with DEPC H₂O and 2.7 μl 2 M NaOAc, pH 5.2 was added. The reaction was diluted with 200 μl PB buffer, put on the purification column, spun 15 sec. at 10,000×g, followed by 3 washes of 500 μl PE buffer (15 sec. at 10,000×g) and eluted two times in 50 μl DEPC H₂O total (1 min. at 10,000×g). Frequency of incorporation and amount of cDNA labeled produced were evaluated for both labeled dCTPs by spectrophotometer (NanoDrop® ND-1000, USA) at 260 nm, 550 nm and 650 nm. The labeled material was dry by speed vacuum (Savant SC110A, USA) and resuspended in 3.75 μl μH₂O total for both the Cy5-dCTP labeled samples and the Cy3-dCTP labeled samples. Finally, a dye swap reaction was performed for each patient on the same day to account for the potential differential incorporation of the Cye-dCTP dyes used in the first strand cDNA labeling reaction.

5. Focused Microarray Preparation

Oligonucleotides 68 nucleotides in length corresponding to targets-of-interest were designed by analyzing sequences showing less than 30% identity base to base with other coding sequences (cds) submitted to NCBI bank. The comparison was done using the Blast research program (Altschul et al. (1990) J. Mol. Biol. 215: 403-410). For the focused microarray, the basic melting point temperature (Tm; salt concentration of 50 mM Na+) of each oligonucleotide was calculated, and averaged 76.69° C.±3.92° C. The total GC content averaged 51.47%±9.75%. Two negative controls (68 bp of antisense cds of the ABpC1 and ABp6 targets) were also synthesized (ABpC1N and ABp6N). Oligonucleotides selection on the focused microarray was based on microarray results obtained on two previous versions of the microarray, and on comparison to 2D proteomic analysis. The complete group of oligonucleotides is shown above in the Detailed Description.

The oligonucleotides were synthesized by Integrated DNA Technologies (IDT, Coralville, Iowa). Quality control was done by MALDI-TOF Mass spectrometry (IDT, Coralville, Iowa). Moreover, an Electrospray MS analysis was done in order to confirm an oligonucleotide's position on the plate used for printing, and represented a second independent verification of oligonucleotide integrity. Electrospray MS analysis can be performed by commercial service providers such as West Coast Analytical Services (Santa Fe Springs, Calif.).

Different dilutions of positive control DNA from Arabidopsis thaliana Chlorophyll Synthetase G4 (from undiluted 200 ng/μl: 1:2, 1:4, 1:8, 1:16, 1:32, 1:64) were printed for each grid and used for the normalization process. Oligonucleotide concentration was normalized to 40 uM in 50% DMSO and printed on UltraGAPS™ (Corning, Corning, N.Y.) by the VersArray CHIP Writer Prosystems (BioRad Laboratories, Hercules, Calif.) with the Stealth Micro Spotting Pins (#SMP3) (Telechem International, Inc., Sunnyvale, Calif.). Each oligonucleotide was printed in triplicate on a grid. Each focused microarray contained three grids. Buffer (50% DMSO) and Salmon Testis DNA (0.2 μg/ml in 50% DMSO; Sigma D-7656, Sigma Corp, St. Louis, Mo.) were also printed for BioChip analysis step. Blank spots were also done and used for analysis steps. After the printing, the slides were dry by overnight incubation in the CHIP Writer chamber, treated by UV Stratalinker (Stratagene, La Jolla, Calif.) at 600 mJoules and baked in an oven for at least 6 to 8 hours.

Printing quality control was done with the PARAGON™ DNA Microarray Quality Control Stain kit (Molecular Probes, Invitrogen Corp., Carlsbad, Calif.) following the manufacturer's recommendations. The slides were scanned with the ScanArray™ Lite MicroArray Scanner (Perkin Elmer, Wellesley, Mass.). The quality control test was done on at least one slide per printing run.

6. Hybridization Conditions

The focused microarray slide was pre-washed before the prehybridization step. First, the microarray was preheated at 42° C. for 20 min in 2×SSC (300 mM NaCl; 30 mM sodium citrate)/0.2% SDS under agitation. The slide was subsequently incubated for 5 min. at room temperature in 0.2×SSC (30 mM NaCl, 3 mM sodium citrate) under agitation and then agitated for 5 min. at room temperature in DEPC H₂O. The slide was spun dry at 1000×g for 5 min. and prehybridized in DIG Easy Hyb Buffer (Roche Diagnostics Corp., Indianapolis, Ind.) containing 0.02% Bovine Serum Albumin (Roche, USA) at 42° C. in humid chamber for 3 hrs then washed two times in DEPC H2O, and once in Isopropanol (Sigma, USA, St. Louis, Mo.) and spun dry at 1000×g for 5 min.

To the mixed Cy3/Cy5 probe prepared as described above, 15 μg Baker tRNA (Roche Diagnostics Corp., Indianapolis, Ind.) and 1 μg Cot-1 DNA (Roche Diagnostics Corp., Indianapolis, Ind.) were added. The probe was incubated 5 min. at 95° C., put on ice for 1 min. and diluted with 14 μl DIG Easy Hyb buffer (Roche Diagnostics Corp., Indianapolis, Ind.). The probe was spun for two min. at 100×g and incubated at 42° C. for at least 5 min.

The three supergrids on the slide were separated by a Jet-Set Quick Dry TOP Coat 101 line (L'OREAL, Paris #FX268). Each probe was added to its respective supergrid and covered by a preheated (42° C.) coverslip (Mandel Scientific Company Ltd., Guelph, ON, CA). The slide was incubated at 42° C. in humid chamber for at least 15 hrs.

The coverslips were removed by dipping in 1×SSC (150 mM NaCl; 15 mM sodium citrate)/0.2% SDS solution preheated at 50° C. Slides were washed 3 times for 5 min./wash in 1×SSC (150 mM NaCl; 15 mM sodium citrate)/0.2% SDS solution preheated at 50° C. with agitation, 3 times in 0.1×SSC (15 mM NaCl; 1.5 mM sodium citrate)/0.2% SDS solution preheated at 37° C. with agitation and once in 0.1×SSC (15 mM NaCl; 1.5 mM sodium citrate) with agitation for 5 min. The slides were dipped several times in DEPC H2O. Slides were then spun dry at 1000×g for 5 min.

7. Statistical Analysis

Focused microarray slides were scanned with the ScanArray™ Lite MicroArray Scanner (Packard BioSciences, Perkin Elmer, Wellesley, Mass.) and the analysis was done with the QuantArray® Microarray Analysis software version 3.0 (Packard BioSciences, Perkin Elmer, Wellesley, Mass.) using the adaptative method.

The QuantArray® data results were analyzed by the spot background subtracted values for Cy5 and Cy3 channels. Spots with signal to noise ratios lower than 1.5 were discarded. The signal intensity was adjusted to a minimum of 100 and spots with signal value lower than 100 in both channels were discarded. Normalization of the ratios with the spike positive control (Arabidopsis thaliana) was done normalize the ratio to one for that control on each slide. The slides were discarded when the negative and/or positive controls failed, i.e., those with high background. The average of the ratios for each target was done between the direct and the reciprocal labeling reactions. Statistical analysis was done with the ArrayStat 1.0 (Imaging Research Inc., St. Catharines, Ontario, CA). A log transformation of the ratio data was followed by a Student T test for two independent conditions using a proportional model without offsets at a p<0.05 thresholds. A significant increase (ratio Cy5/Cy3 higher than 2.0) or decrease (ratio Cy5/Cy3 lower than 0.5) are considered to be significant if the p value is lower than 0.05.

8. Classification Algorithms

Class prediction analyses were performed using the BRB ArrayTools developed by Dr. Richard Simon (NIH/NCI) and Amy Peng. Briefly, class prediction analyses were done on the results obtained for each patient in the study. Patients were divided into two classes following their malignancy: normal class and tumor class. These classes became the training sets by which patients were compared for purposes of classification. The classification algorithms used the expression data from the training sets to make all patient classifications during the tests. Class determination was done based on the clinical data associated to each patient. There were six different classification algorithms used in the studies: compound covariate predictor, diagonal linear discriminant analysis, nearest neighbor predictor (1-NN and 3-NN), nearest centroid predictor and support vector machine predictor. Those analyses permitted the development of a multi-gene classifier to predict the class for a new sample and estimate the misclassification rates. Cross-validation of the class prediction classifiers were done by the leave one-out study and permutation tests (n=2000) were conducted to address significance of the cross-validation test error rate. FIGS. 14A and 14B show the levels of expression for cathepsin D in breast cancer patients. The data showed that cathepsin D could be utilized individually to identify cancer patients. FIG. 15A showed the use of all marker genes to classify tumors. FIG. 15B removed HER-2 from the analysis, improving the results of the test.

### Example 3

### Classification of Cell Samples Isolated From Breast Normal Subjects and Breast Cancer Patients Using Quantitative RT-PCR

1. Patient Samples and Normal Samples

Patient material was purchased from Asterand, Inc. (Detroit, Mich.), Clinomics Biosciences, Inc (Watervliet, N.Y.) and Biochain Institute, Inc. (Hayward, Calif.). Seventy-two patients were tested in Q-PCR. Of the 123 samples obtained from those patients, 56 were normal breast samples and 67 were breast tumors. Standard clinical and pathological reports were available for each cancer patient included in this study. The majority were post-menopausal patients. Most breast cancer samples were infiltrating ductal carcinoma of the breast. Different histological stages were represented but the majority of the patients were stage II, with lymph node negative status. Patients included in this study had tumor content greater than 70%.

Total RNA was extracted from snap frozen tissue samples of Asterand, Inc., Clinomics Biosciences, Inc. or Biochain Institute, Inc. with the Trizol Reagent kit (Gibco-BRL, Carlsbad, Calif.) using the recommended extraction procedures. Total RNA was treated with RNA-free DNAse I (New England BioLabs, Beverly, Mass.), and was purified with the RNEasy kit (Qiagen, Hilden, Germany). RNA samples were visualized on an Agilent 2100 BioAnalyzer (Agilent Technologies, Foster City, Calif.) for purity and integrity.

2. RNA Primer Design and Testing

The design of the primers used during for Quantitative RT-PCR required analysis to ensure proper annealing would occur so as to provide proper products. Briefly, several potential primer pairs were analyzed to determine potential hairpin formation and formation of dimer pairs using PrimerQuest® software according to manufacturer's instructions (Integrated DNA Technologies, Coralville, Iowa). It was determined that all primers had to provide amplification products of between 80-200 bp, and have melting temperatures between 57° C. and 61° C. in the case of sample primers and between 62° C. and 67° C. in the case of Taqman® primers.

Upon determination of the appropriate primer pairs to use for the Quantitative RT-PCR analysis, the optimal concentration for each primer pair was determined. Briefly, RT-PCR was performed as described below using all primer pairs at varying concentrations between 100 nM and 300 nM. Ct values were determined for each concentration as described below. Table 9 shows the primers used for the analysis.

TABLE 9

| | | | |
|---|---|---|---|
| Marker | Forward Primer | Reverse Primer | Quantity |

| | | | |
|---|---|---|---|
| Keratin 19 | 5′TGCTGGATGAGCTGACCCT3′ | 5′CGGCTGGTGAACCAIGCT3′ | 100 nM |
| Ezrin | 5′GCAGGACTATGAGGAGAAGACAAAGA3′ | 5′GCTCCTGGCTCTTTATCTGATCCA3′ | 100 nM |
| S1c9a3r1 | 5′AGGATCGCATTGTGGAGGTGAA3′ | 5′CCTGAGATGGGATCACTCTGCATT3′ | 100 nM |
| Cathepsin D | 5′AAGCTGGGAGGCAAAGGCTACAAG3′ | 5′CACCCTGTTGTTGTCACGGTCAAA3′ | 100 nM |
| ACRAB-PII | 5′TGGACCAGAGAACTGACCAACG3′ | 5′CTCACTCTCGAACGTAGACCCT | 100 nM |

3. Total RNA Reaction Mixtures Utilized in the Present Study

One hundred nanograms of total RNA, as measured by the NanoDrop® ND-1000 spectrophotometer (NanoDrop Technologies, Wilmington, Del.), was used for reverse transcription into cDNA. Briefly, tRNA was mixed with 500 ng oligodT₁₈, 250 ng pdN₆ random primers, and 10 pg Arabidopsis RNA (internal control) to a final volume of 10 μl. The mixture was heated 10 min. at 65° C. and quickly transferred to ice for two min. The cDNA synthesis solution (50 mM Tris-HCl, pH 8.3; 75 mM KCL; 3 mM MgCl₂; 10 mM DTT; 1 mM dNTP; 200 unit of SSIII enzyme) was added and incubated for 5 min. at 25° C. followed by 1 hr. and 30 min. at 50° C. Finally, the mixture was centrifuged, heated for 5 min. at 95° C., and centrifuged a second time. The samples were placed on ice until further use.

4. First Strand cDNA Reaction

Quantitative RT-PCR experiments were done on a Stratagene Mx3005 QPCR system (Straragene, La Jolla, Calif.) using SYBRGREEN (Stratagene, La Jolla, Calif.) binding dye. Specific primer sets were designed using the Primer Quest software (Integrated DNA Technologies, Coralville, Iowa) (see Table 7 for primers). For each transcript set, the optimum concentration was determined and standard curves were generated using six dilutions of a cDNA sample from a normal pool. To confirm that the amplification occurred on the targeted sequences, the dissociation curves were examined for the presence of a single sharp peak at the melting temperature of the amplicon. Typical standard curve included 0, ⅕, 1/10, 1/25, 1/50, 1/250>, and 1/500 dilutions of cDNA (Breast normal pool).

The cDNA( 1/25) was mixed with Brillant Quantitative RT-PCR MasterMix solution (Stratagene, La Jolla, Calif.), which included 100 nM of each primer (forward and reverse). The mixture was placed in 96-wells plate. PCR reactions consisted of 40 cycles of denaturation for 30 seconds at 95° C., 40 cycles of annealing for 1 min. at 60° C., and 40 cycles of extension for 30 seconds at 72° C., after an initial denaturation step at 95° C. for 10 min. Experiment were performed in duplicate for each data point and repeated twice. The Quantitative RT-PCR experiments were performed with the arabidopsis internal control to determine the efficiency of the PCR reaction. A standard curve of normal breast pool was also compared to the breast patient samples.

5. Standard Curves

The dilution sets of BRpool used for the standard curve were based on preliminary results were serial dilutions of cDNA of the BRpool were used with all the ABp targets in a Real-time PCR reaction. The dilution sets are <⅕>or < 1/10>, < 1/50>, < 1/250>and < 1/500>. The dilutions sets that, all together, were giving efficiency between 90 and 110% were chosen. Eighteen PCR runs (19 for Arabidopsis) were performed to screen all the patients twice. Table 4 presents an average of the reaction efficiency obtained for each target during these 18 PCR runs. On certain occasions, dilution sets or replicates had to be removed to obtain an acceptable curve. PCR efficiency for each primer pair was determined.

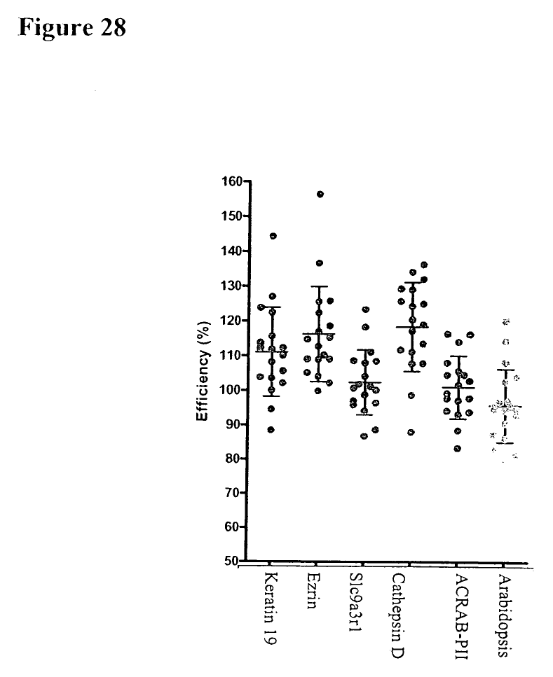

A primer pair was considered “perfect” if the efficiency is between 90 and 110%. A lower efficiency indicated un-optimized primers and higher efficiency, the presence of inhibitors or tRNA degradation. S1c9a3r1, ACRAB-PII, and Arabidopsis control primers had appropriate efficiency and only Arabidopsis had CV % higher than 10%. All these standard curves had R square values higher than 0.98 and less than 2.8% of the dilution sets were rejected. Keratin 19, ezrin, and cathepsin D primers had efficiencies between 110 and 120%. As the other primers have acceptable efficiency, the presence of inhibitors could not be associated to these higher percentages. The CVs of these primers are also higher than 10 but lower than 12%. FIG. 28 shows the efficiency results of each primer pair. The results indicate that the primers have efficiencies that are within the acceptable ranges.

6. Statistical Analysis

At the end of each run, the computer provided all PCR amplification curves. The Ct value was used for the analysis of the results. This value corresponded to the cycle number, in the linear phase of the amplification, where the fluorescence reaches a given threshold. The amount of target at this cycle was given by Equation 1:
Xₙ=X₀×2ⁿ n=number of cycles

The comparative evaluation, based on the cycle threshold (Ct) of the gene of interest normalize to the arabidopsis control, was used to evaluate the relative gene expression.

The ratio (sample/BRpool) was determined following the equation of Pfaffl that takes into consideration the efficiency of the PCR reaction (Paffl 2001):

$$Ratio = \frac{{( 1 + {E}_{target} )}^{dCttarget ( Control - Sample )}}{{( 1 + {E}_{ref} )}^{dCtref ( Control - Sample )}}$$

Ratio: sample/BRpool
E: efficiency of the PCR reaction for the given primer set (based on the slope of the standard curve)
Target: ABp target
Control: BRpool
Ref: Spiked Arabidopsis
dCt: Ct of the control (BRpool)—Ct of the target
   5. Results

The results of the validation of focused microarray data are described in FIGS. 19-25. The marker genes analyzed by quantitative RT-PCR showed increased ratios of expression in tumor samples as compared to the normal tissues of the BRPool (FIGS. 19-25). These results corresponded to the results obtained using the focused microarray (FIGS. 19-25). Of particular note, ACRAB-PII, s1c9a3r1, and cytokeratin 19 showed a large increase in the levels of expression in patient samples as compared to the levels of expression found in normal breast tissue samples (FIGS. 19, 21, and 23).

### Example 4

### Classification of Cell Samples Isolated From Breast Normal Subjects and Breast Cancer Patients Using RT-PCR

1. Experimental Procedures

All experiments were performed as described in Example 3. Seventy-two patient samples were obtained as described in Example 3. Of the 72 samples, 29 were normal breast samples and 43 were breast tumors. Standard clinical and pathological reports were available for each cancer patient included in this study. The mean age of the patients was 53.5 years, with a standard deviation of 12.3. The majority were post-menopausal patients. Most breast cancer samples (76%) were infiltrating ductal carcinoma of the breast. Different histological stages were represented but the majority of the patients were stage II, with lymph node negative status. Patients included in this study had tumor content greater than 70%.

2. Results

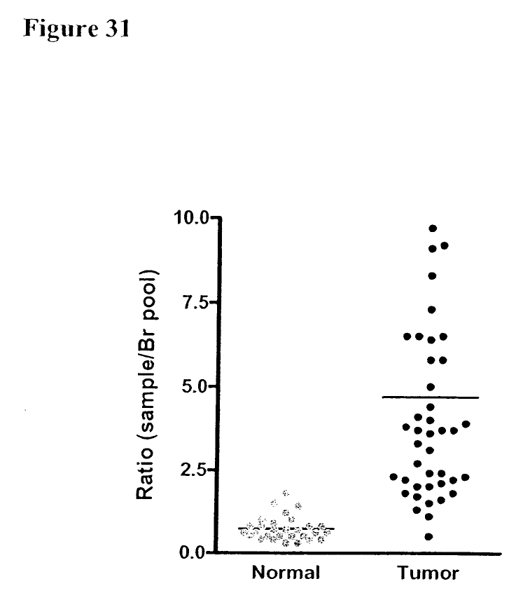

The results of the validation of focused microarray data are described in FIGS. 31-40. Quantitative RT-PCR experiments showed that tumor samples showed significant increases in the ratios of expression of marker genes cathepsin D, s1c9a3r1, ezrin, ACRAB-PII, and keratin 19 as compared to the normal tissue samples of BRPool (FIGS. 31-35). Normal tissues did not show an increase in the ratio of expression of the marker genes as compared to the normal tissues of the BRPool (FIGS. 31-35).

These results were similar to the microarray results (FIGS. 34-38). All marker genes displayed significant increased ratios of expression in tumor samples as compared to the BRPool, which was shown by the focused microarray results (Table 8). Fold increased ratios in tumor samples as compared to the normal tissues in the BRPool ranged from 2.9 to 6.4 fold, which corresponded for the five markers analyzed with the results found using the focused microarray (Table 8). Of particular note, cathepsin D, s1c9a3r1, and cytokeratin 19 showed a large increase in the levels of expression in patient samples as compared to the levels of expression found in normal breast tissue samples (Table 8).

### Example 5

### Classification of Cell Samples Isolated From Ovarian Normal Subjects and Ovarian Cancer Patients Using RT-PCR

1. Patient Samples and Normal Samples

Patient material is purchased from Asterand, Inc. (Detroit, Mich.), Clinomics Biosciences, Inc (Watervliet, N.Y.) and Biochain Institute, Inc. (Hayward, Calif.). For the ovarian cancer groups, only patients with greater than 70% tumor cell content in the tumor mass are included in the studies. Total RNA is extracted from snap frozen tissue samples with the Trizol Reagent kit (Gibco-BRL, Carlsbad, Calif.) using the recommended extraction procedures of Asterand, Inc., Clinomics Biosciences, Inc. or Biochain Institute, Inc. Total RNA is treated with RNA-free DNAse I (New England BioLabs, Beverly, Mass.) and is purified with the RNEasy kit (Qiagen, Hilden, Germany). RNA samples are visualized on an Agilent 2100 BioAnalyzer (Agilent Technologies, Foster City, Calif.).

Each patient included in the study is screened against the same normal total RNA pool in order to compare them together. Ovarian normal total RNA pools are purchased from Biochain Institute, Inc. (Hayward, Calif.). The normal ovarian subjects and the ovarian cancer patients are screened in triplicate.

2. Total RNA Controls Utilized in the Present Study

Total RNA received from the three suppliers described above is quantified with the NanoDrop® ND-1000 spectrophotometer (NanoDrop Technologies, Wilmington, Del.), and the ratio A₂₆₀/A₂₈₀ is calculated. Total RNA with an A₂₆₀/A₂₈₀ ratio between 1.9-2.3 in 10 mM Tris, pH 7.5 is included in the study. Integrity of the ribosomal RNA is visualized by standard 1 % agarose gel electrophoresis in TBE buffer (Tris 9 mM; 9 mM borate acid; 0.2 mM EDTA) containing 0.04% EtBr. RNA samples without apparent RNA degradation, which is indicated by appropriate rRNA 28S/18S ratios, are used for the RT-PCR reaction.

3. First Strand cDNA Reaction

The following procedure is based on Invitrogen's protocol. Briefly, 10 μl DEPC-H2O is added to the RNA pellet. 1 μl RNase inhibitor is then added and the pellet is resuspended with gentle tapping. Reverse Transcription is carried out with the SuperScript First-Strand Synthesis System for RT-PCR. The following RNA/primer mixture is prepared. Total RNA is added to a reaction tube so that the total RNA is 5 mg. To the total RNA is added a mixture of random hexamers (50 ng/ml). 3 ml of 10 mM dNTP mix is added and the solution is gently mixed by inversion. The total volume of the solution is then brought to 10 ml by the addition of DEPC-dH₂O.

The samples are incubated at 65° C. for 5 min., and then on ice for at least 1 min. The reaction is prepared for each reaction (2 ml of 10×RT buffer, 4 ml of 25 mM MgCl₂, 2 ml of 0.1 M DTT, 1 ml of RNAaseOUT (Invitrogen Corp., Carlsbad, Calif.). The reaction mixture is added to the RNA/primer mixture, which is mixed briefly by inversion. The mixture is placed at room temperature for 2 min. Subsequently, 1 ml (50 units) of SuperScript II RT (Invitrogen Corp., Carlsbad, Calif.) is added to each tube. Tubes are mixed and incubated at 25° C. for 10 min. The tubes are then incubated at 42° C. for 50 min, and the heat inactivated at 70° C. for 15 min. Tubes are chilled on ice for at least 5 min. To each tube is added 1 ml RNase H. Tubes are incubated at 37° C. for 20 min. The first strand reaction is stored at −20° C. until use for real-time PCR.

4. Real-time PCR

Primer concentrations are normalized using spectrophotometry to determine the A₂₆₀ counts for the DNA. The gene-specific forward and reverse primer pair are mixed for each marker gene of interest using sequence specific information that is determined by the BLAST sequence alignment protocol (Altschul et al. (1990) J. Mol. Biol. 215: 403-410). Each primer (forward or reverse) concentration in the mixture is 5 pmol/ml. The PCR program is run on an ABI Prism SDS 7000. The following protocol is used: 1) 50° C. 2 min, 1 cycle, 2) 95° C. 10 min 1 cycle, 3) 95° C. 15 s→60° C. 30 s→72° C. 30 s, 40 cycles, and 4) 72° C. 10 min, 1 cycle. The real-time PCR reaction mixture is 50 ml and is prepared in optical tubes for each marker gene. In each real time PCR reaction tube is added 25 ml of SYBR Green Mix (2×), 0.5 ml of liver cDNA, 2 ml of the primer pair mix (5 pmol/ml each primer), 22.5 ml of ddH₂O. After PCR is finished, the tubes are removed from the machine. The PCR specificity is examined by 3% agarose gel using 5 ml from each reaction. The tubes are placed back in the PCR machine and the dissociation curve analysis is performed. The real-time PCR result is analyzed with the SDS 7000 software using manufacturer's protocol.

7. Statistical Analysis

Statistical analysis of the levels of expression of marker genes is performed using the Student T test for two independent experiments at a p<0.05 threshold. The results are used to determine the levels of expression for the marker genes in a ovarian cancer patient population and a normal ovarian population.

8. Classification Algorithms

Class prediction analyses were performed using the BRB ArrayTools developed by Dr. Richard Simon (NIH/NCI) and Amy Peng. Briefly, class prediction analyses were done on the results obtained for each patient in the study. Patients were divided into two classes following their malignancy: normal class and tumor class. These classes became the training sets by which patients were compared for purposes of classification. The classification algorithms used the expression data from the training sets to make all patient classifications during the tests. Class determination was done based on the clinical data associated to each patient. There were six different classification algorithms used in the studies: compound covariate predictor, diagonal linear discriminant analysis, nearest neighbor predictor (1-NN and 3-NN), nearest centroid predictor and support vector machine predictor. Those analyses permitted the development of a multi-gene classifier to predict the class for a new sample and estimate the misclassification rates. Cross-validation of the class prediction classifiers were done by the leave one-out study and permutation tests (n=2000) were conducted to address significance of the cross-validation test error rate. Ovarian cancer patients are appropriately classified using the levels of expression for cathepsin D. Ovarian cancer patients are also appropriately classified using the levels of expression cathepsin D in combination with other classifiers identified in Tables 5 and 6 above.

### EQUIVALENTS

Those skilled in the art will recognize, or be able to ascertain, using no more than routine experimentation, numerous equivalents to the specific compositions and procedures described herein. Such equivalents are considered to be within the scope of this invention, and are covered by the following claims.

1. A method of diagnosing cancer in a subject, comprising:
(a) providing a focused microarray, the microarray having a plurality of nucleic acid capture probes, each capture probe being complementary to a marker gene selected from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, ATP synthase δ, PDI/ER-60 precursor, ATP synthase β, prostasin, cathepsin β, FAS, HSCP60, topoisomerase IIα, PCNA, ezrin, PDI, cathepsin D, A-CRABP II, HSC70, rad 23 homolog β, ETF3 subunit 2β, proteosome B1 subunit proprotein, β-tubulin, s1c9a3r1, prosolin, HSP60, HER-2, L-plastin, estrogen receptor α, HSP27, thioredoxine peroxidase I, calumenin, 14-3-3 eta chain, Ki 67, MRP1, “similar to stratifin,” UCHL-1, mammaglobin 2, cellular RNA binding protein, p53, and annexin I;
(b) detecting a level of expression in a cell sample of the selected marker genes by contacting the nucleic acid capture probes with nucleic acids from the cell sample so as to allow for the hybridization of the nucleic acid capture probes with the nucleic acids from the cell sample; and
(c) comparing the level of expression of the selected marker genes in the cell sample to the level of expression of the same marker genes in a normal cell sample of the same tissue type,
wherein the presence of a cancer cell is indicated if the level of expression of five or more of the selected marker genes in the cell sample is greater than the level of expression of the same marker genes in the normal cell sample of the same tissue type.

2. The method of claim 1, wherein the microarray has a plurality of nucleic acid capture probes that are complementary to marker genes selected from the group consisting of cytokeratin 18, cytokeratin 7, keratin 19, α-enolase, s1c9a3r1, TPI, and HER-2.

3. The method of claim 2, wherein the plurality of nucleic acid capture probes is at least six.

4. The method of claim 2, wherein the plurality of nucleic acid capture probes is at least seven.

5. The method of claim 1, wherein the presence of a cancer cell is indicated if the level of expression of six or more of the selected marker genes in the cell sample is greater than the level of expression of the same marker genes in the normal cell sample of the same tissue type.

6. The method of claim 1, wherein the presence of a cancer cell is detected if the level of expression of seven or more of the selected marker genes in the cell sample is greater than the level of expression of the same marker genes in the normal cell sample of the same tissue type.

7. The method of claim 1, wherein the plurality of marker genes selected is at least six, and an increased level of expression of at least five marker genes in the cell sample compared to the level of expression in the normal cell sample of the same tissue type indicates that the cell sample is neoplastic.

8. The method of claim 1, wherein the plurality of marker genes selected is at least seven, and an increased level of expression of at least five marker genes in the cell sample compared to the level of expression in the normal cell sample of the same tissue type indicates that the cell sample is neoplastic.

9. The method of claim 1, wherein the plurality of marker genes selected is at least eight, and an increased level of expression of at least six marker genes in the cell sample compared to the level of expression in the normal cell sample of the same tissue type indicates that the cell sample is neoplastic.

10. The method of claim 1, wherein the cell sample is from breast tissue.

11. The method of claim 10, wherein the plurality of marker genes is keratin 19, s1c9a3r1, FABP 4, and HER-2.

12. The method of claim 1, wherein the cell sample is from ovarian tissue and the plurality is at least three marker genes selected from the group consisting of cytokeratin 18, cytokeratin 7, α-enolase, and TPI.

13. The method of claim 12, wherein the plurality is cytokeratin 18, cytokeratin 7, α-enolase, and TPI.

14. The method of claim 1, wherein the step of comparing the level of expression of the selected marker genes further comprises using a class prediction algorithm to differentiate the level of expression of the selected marker genes in the cell sample from the level of expression of the same marker genes in the normal cell sample of the same tissue type.

15. The method of claim 14, wherein the one or more class prediction algorithms is selected from the group consisting of compound covariate predictor, diagonal linear discriminant analysis, nearest neighbor predictor, nearest centroid predictor, and support vector machine predictor.

16. The method of claim 1, wherein the presence of a cancer cell is indicated if the level of expression in the cell sample of at least one of the selected marker genes is at least two times the level of expression of the same marker gene in the normal cell sample of the same tissue type.

17. The method of claim 1, wherein the presence of a cancer cell is indicated if the level of expression in the cell sample of at least one of the selected marker genes is at least three times the level of expression of the same marker gene in the normal cell sample of the same tissue type.

18. The method of claim 1, wherein the presence of a cancer cell is indicated if the level of expression in the cell sample of at least one of the selected marker genes is at least four times the level of expression of the same marker gene in the normal cell sample of the same tissue type.

19. The method of claim 1, wherein the presence of a cancer cell is indicated if the level of expression in the cell sample of at least one of the selected marker genes is at least five times the level of expression of the same marker gene in the normal cell sample of the same tissue type.

20. The method of claim 1, wherein the presence of a cancer cell is indicated if the level of expression in the cell sample of at least one of the selected marker genes is at least six times the level of expression of the same marker gene in the normal cell sample of the same tissue type.

21. Any of the methods of claims 16-20, wherein the step of comparing the level of expression of the selected marker gene(s) comprises using one or more class prediction algorithms selected from the group consisting of compound covariate predictor, diagonal linear discriminant analysis, nearest neighbor predictor, nearest centroid predictor, and support vector machine predictor.

22. The method of claim 1, wherein the subject is a human.

23. The method of claim 1, wherein the presence of cancer being detected is selected from the group consisting of ovarian carcinoma, serous adenocarcinoma, clear cell adenocarcinoma, endometrioid carcinoma, mucinous adenocarcinoma, breast adenocarcinoma, and infiltrating ductal carcinoma.

24. A method of diagnosing breast cancer in a subject, comprising:
(a) selecting at least six marker genes from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, PDI/ER-60 precursor, ATP synthase β, prostasin, cathepsin β, FAS, HSCP60, topoisomerase IIα, PCNA, ezrin, PDI, cathepsin D, A-CRABP II, HSC70, β-tubulin, s1c9a3r1, prosolin, HSP60, HER-2, L-plastin, estrogen receptor α, HSP27, thioredoxine peroxidase I, calumenin, GAPDH, 14-3-3 eta chain, and annexin I;
(b) detecting a level of expression of the selected marker genes in a breast cell sample by contacting probes capable of binding or hybridizing with the marker genes isolated from the cell sample; and
(c) comparing the level of expression of the selected marker genes in the breast cell sample to a level of expression of the same marker genes detected in a normal breast cell sample,
wherein the presence of breast cancer is indicated if the level of expression of three or more marker genes in the breast cell sample is greater than the level of expression for in the normal breast cell sample.

25. The method of claim 24, wherein at least six marker genes are selected, and an increased level of expression of a plurality of at least five marker genes in the breast cell sample compared to the level of expression in the normal breast cell sample indicates the presence of breast cancer.

26. The method of claim 24, wherein at least seven marker genes are selected, and an increased level of expression of at least six marker genes in the breast cell sample compared to the level of expression in the normal breast cell sample indicates the presence of breast cancer.

27. The method of claim 24, wherein the plurality of marker genes comprises keratin 19, s1c9a3r1, and HER-2.

28. The method of claim 24, wherein the level of expression of marker genes is detected by nucleic acid capture probes attached to a solid support.

29. The method of claim 24, wherein the breast cancer is a breast adenocarcinoma or an infiltrating ductal carcinoma.

30. The method of claim 24, wherein the subject is a human.

31. The method of claim 24, wherein the presence of cancer is indicated if the level of expression in the breast cell sample of at least one of the selected marker genes is at least two times the level of expression of the same marker gene in the normal breast cell sample.

32. The method of claim 24, wherein the marker genes are selected from the group consisting of cathepsin D, ezrin, keratin 19, s1c9a3r1, A-CRABP II, HER-2, and estrogen receptor α.

33. The method of claim 32, wherein the presence of cancer is indicated if the level of expression in the breast cell sample of at least one of the selected marker genes is at least three times the level of expression of the same marker genes in the normal breast cell sample.

34. The method of claim 33, wherein the average level of expression is determined in a breast cell sample and a normal breast cell sample for at least three marker genes selected from the group consisting of cathepsin D, ezrin, keratin 19, s1c9a3r1, A-CRABP II, HER-2, and estrogen receptor α.

35. The method of claim 34, wherein the level of expression of the marker genes in the breast cell sample is compared to the level of expression of the same marker genes in the normal breast cell sample using one or more class prediction algorithms selected from the group consisting of compound covariate predictor, diagonal linear discriminant analysis, nearest neighbor predictor, nearest centroid predictor, and support vector machine predictor.

36. The method of claim 34, wherein the marker genes are keratin 19, s1c9a3r1, and HER-2.

37. The method of claim 36, wherein the presence of cancer is indicated if the level of expression in the breast cell sample of at least one of the plurality of selected marker genes is at least four times the level of expression of the same marker genes in the normal breast cell sample.

38. The method of claim 24, wherein the marker genes are selected from the group consisting of keratin 19, s1c9a3r1, HER-2, and FABP 4.

39. The method of claim 38, wherein the presence of cancer is indicated if the level of expression of keratin 19, s1c9a3r1, and HER-2 is at least four times greater in the breast cell sample than the level of expression for the same marker genes in the normal breast cell sample and the level of expression of FABP 4 is decreased in the breast cell sample as compared to the normal breast cell sample.

40. The method of claim 24, wherein comparing the level of expression of the selected marker genes further comprises using a class prediction algorithm to differentiate the level of expression of the selected marker genes in the cell sample from the level of expression of the same marker genes in the normal cell sample of the same tissue type.

41. The method of claim 40, wherein comparing the level of expression of the selected marker genes further comprises differentiating the level of expression of the selected marker genes in the cell sample from the level of expression of the same marker genes in the normal cell sample of the same tissue type using one or more class prediction algorithms selected from the group consisting of compound covariate predictor, diagonal linear discriminant analysis, nearest neighbor predictor, nearest centroid predictor, and support vector machine predictor.

42. The method of claim 24, wherein the level of expression of marker genes is determined using RT-PCR, PCR, nucleic acid blotting, dot blotting, or microarray.

43. A method of diagnosing ovarian cancer in a subject, comprising:
(a) selecting a plurality of marker genes from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, PDI/ER-60 precursor, ATP synthase β, prostasin, cathepsin β, FAS, HSCP60, topoisomerase IIα, PCNA, ezrin, PDI, cathepsin D, A-CRABP II, and annexin I;
(b) detecting a level of expression of the selected marker genes in an ovarian cell sample by contacting probes capable of binding or hybridizing with the marker genes isolated from the cell sample; and
(c) comparing the level of expression of the selected marker genes in the ovarian cell sample to a level of expression of the same marker genes detected in a normal ovarian cell sample,
wherein the presence of ovarian cancer is indicated if the level of expression of two or more marker genes in the ovarian cell sample is greater than the level of expression of the same marker genes in the normal ovarian cell sample.

44. The method of claim 43, wherein the plurality of marker genes selected is at least six, and an increased level of expression of a plurality of at least four marker genes in the ovarian cell sample compared to the level of expression in the normal ovarian cell sample indicates the presence of ovarian cancer.

45. The method of claim 43, wherein the plurality of marker genes selected is at least seven, and an increased level of expression of a plurality of at least five marker genes in the ovarian cell sample compared to the level of expression in the normal ovarian cell sample indicates the presence of ovarian cancer.

46. The method of claim 43, wherein at least three marker genes selected from the group consisting of cytokeratin 18, cytokeratin 7, TPI, and α-enolase.

47. The method of claim 43, wherein the marker genes are cytokeratin 18, cytokeratin 7, TPI, and α-enolase.

48. The method of claim 43, wherein the step of comparing the level of expression of marker genes in the ovarian cell sample to the level of expression of the marker genes in the normal ovarian cell sample comprises using one or more class prediction algorithms selected from the group consisting of compound covariate predictor, diagonal linear discriminant analysis, nearest neighbor predictor, nearest centroid predictor, and support vector machine predictor.

49. The method of claim 43, wherein the level of expression of marker genes is detected by nucleic acid capture probes attached to a solid support.

50. The method of claim 43, wherein the ovarian cancer cell is an ovarian carcinoma, a serous adenocarcinoma, a clear cell adenocarcinoma, an endometrioid carcinoma, or a mucinous adenocarcinoma.

51. The method of claim 43, wherein the subject is a human.

52. The method of claim 43, wherein the presence of cancer is indicated if the level of expression in the ovarian cell sample of at least one of the selected marker genes is at least three times the level of expression of the same marker gene in the normal ovarian cell sample.

53. The method of claim 43, wherein the marker genes are selected from the group consisting of HDGF, cytokeratin 18, α-enolase, GAPDH, GST-π, TPI, keratin 19, cytokeratin 7, pyrophosphatase inorganic, ATP synthase β, prostasin, cathepsin β, cathepsin D, and A-CRABP II.

54. The method of claim 54, wherein the presence of cancer is indicated if the level of expression in the ovarian cell sample of at least one of the selected marker genes is at least four times the level of expression of the same marker genes in the normal ovarian cell sample.

55. The method of claim 54, wherein the presence of cancer is indicated if a level of expression in the ovarian cell sample of at least two marker genes is at least four times the level of expression for the same marker genes in the normal ovarian cell sample.

56. The method of claim 43, wherein at least five marker genes are selected from the group consisting of HDGF, cytokeratin 18, α-enolase, GAPDH, GST-π, TPI, keratin 19, cytokeratin 7, and A-CRABP II.

57. The method of claim 56, wherein the presence of cancer is indicated if the level of expression in the ovarian cell sample of at least one of the plurality of marker genes is at least five times the level of expression of the same marker genes in the normal ovarian cell sample.

58. The method of claim 56, wherein the presence of cancer is indicated if levels of expression are determined for all marker genes in the ovarian cell sample and the normal ovarian cell sample, and the levels of expression for all marker genes in the ovarian cell sample are at least five times the levels of expression for the same marker genes in the normal ovarian cell sample.

59. The method of claim 58, wherein the step of comparing the levels of expression of marker genes in the ovarian cell sample to the levels of expression of the marker genes in the normal ovarian cell sample comprises using a class prediction algorithm.

60. The method of claim 43, wherein the marker genes are selected from the group consisting of cytokeratin 18, α-enolase, TPI, and cytokeratin 7.

61. The method of claim 60, wherein the presence of cancer is indicated if the level of expression in the ovarian cell sample of at least one of the plurality of selected marker genes is at least six times the level of expression of the same marker genes in the normal ovarian cell sample.

62. The method of claim 60, wherein the presence of cancer is indicated if levels of expression are determined for all marker genes in the ovarian cell sample and the normal ovarian cell sample, and the levels of expression for all marker genes in the ovarian cell sample are at least five times the levels of expression for the same marker genes in the normal ovarian cell sample.

63. The method of claim 62, wherein the step of comparing the levels of expression of marker genes in the ovarian cell sample to the levels of expression of the marker genes in the normal ovarian cell sample comprises using a class prediction algorithm.

64. The method of claim 43, wherein comparing the level of expression of the selected marker genes further comprises using a class prediction algorithm to differentiate the level of expression of the selected marker genes in the ovarian cell sample from the level of expression of the same marker genes in the normal ovarian cell sample.

65. The method of claim 57, wherein comparing the level of expression of the selected marker genes further comprises differentiating the level of expression of the selected marker genes in the ovarian cell sample from the level of expression of the same marker genes in the normal ovarian cell sample using one or more class prediction algorithms selected from the group consisting of compound covariate predictor, diagonal linear discriminant analysis, nearest neighbor predictor, nearest centroid predictor, and support vector machine predictor.

66. The method of claim 43, wherein the level of expression of marker genes is determined using RT-PCR, PCR, nucleic acid blotting, dot blotting, or microarray.

67. A focused microarray for diagnosing a neoplasm, comprising:
a) a first set of nucleic acid capture probes comprising a plurality of nucleic acid capture probes, each capture probe being complementary to a marker gene selected from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, ATP synthase δ, PDI/ER-60 precursor, ATP synthase β, prostasin, cathepsin β, FAS, HSCP60, topoisomerase IIα, PCNA, ezrin, PDI, cathepsin D, A-CRABP II, HSC70, rad 23 homolog β, ETF3 subunit 2β, proteosome BI subunit proprotein, β-tubulin, s1c9a3r1, prosolin, HSP60, HER-2, L-plastin, estrogen receptor α, HSP27, thioredoxine peroxidase I, calumenin, 14-3-3 eta chain, Ki 67, MRP1, “similar to stratifin,” UCHL-1, mammaglobin 2, cellular RNA binding protein, p53, and annexin I;
b) a second set of nucleic acid capture probes comprising a plurality of nucleic acid capture probes, each capture probe being complementary to a marker gene encoding an endogenous housekeeping gene;
c) a solid support to which the first and second set of nucleic acid capture probes are attached at predetermined positions.

68. The focused microarray of claim 67, wherein the first set of capture probes is complementary to at least three of the marker genes selected from the group consisting of cytokeratin 7, cytokeratin 18, TPI, α-enolase, keratin 19, s1c9a3r1, and HER-2.

69. The focused microarray of claim 67, wherein the first set of capture probes is complementary to at least four of the marker genes selected from the group consisting of cytokeratin 7, cytokeratin 18, TPI, α-enolase, keratin 19, s1c9a3r1, and HER-2.

70. The focused microarray of claim 67, wherein the first set of capture probes is complementary to at least five marker genes selected from the group consisting of cytokeratin 7, cytokeratin 18, TPI, α-enolase, keratin 19, s1c9a3r1, and HER-2.

71. The focused microarray of claim 67, wherein the first set of capture probes is complementary to at least six of the marker genes selected from the group consisting of cytokeratin 7, cytokeratin 18, TPI, α-enolase, keratin 19, s1c9a3r1, and HER-2.

72. The focused microarray of claim 67, wherein at least one capture probe of the second set is complementary to a marker gene selected from the group consisting of EF-2 and EIF-4B.

73. The focused microarray of claim 67, wherein the second set of capture probes consists of the marker genes EF-2 and EIF-4B.

74. The focused microarray of claim 67, wherein the first set of capture probes is complementary to marker genes selected from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, PDI/ER-60 precursor, ATP synthase β, prostasin, cathepsin β, FAS, HSCP60, topoisomerase IIα, PCNA, ezrin, PDI, cathepsin D, A-CRABP II, HSC70, β-tubulin, s1c9a3r1, prosolin, HSP60, HER-2, L-plastin, estrogen receptor α, HSP27, thioredoxine peroxidase I, calumenin, GAPDH, 14-3-3 eta chain, and annexin I.

75. The focused microarray of claim 67, wherein the first set of capture probes is complementary to marker genes selected from the group consisting of keratin 19, s1c9a3r1, and HER-2.

76. The focused microarray of claim 67, wherein the first set of capture probes is complementary to marker genes selected from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, PDI/ER-60 precursor, ATP synthase β, prostasin, cathepsin β, FAS, HSCP60, topoisomerase IIα, PCNA, ezrin, PDI, cathepsin D, A-CRABP II, and annexin I.

77. The focused microarray of claim 67, wherein the first set of capture probes is complementary to marker genes selected from the group consisting of cytokeratin 18, cytokeratin 7, TPI, and cytokeratin 7.

78. A kit for diagnosing cancer in a subject, comprising:
a) a first set of probes for the detection of one or more marker genes selected from the group consisting of HDGF, cytokeratin 18, α-enolase, GAPDH, GST-π, TPI, 5C5-2, prohibitin, keratin 19, cytokeratin 7, HnRNP, pyrophosphatase inorganic, BIP, CBX3, ATP synthase δ, PDI/ER-60 precursor, ATP synthase β, prostasin, cathepsin β, FAS, HSCP60, topoisomerase IIα, PCNA, ezrin, PDI, cathepsin D, A-CRABP II, HSC70, rad 23 homolog β, ETF3 subunit 2β, proteosome B1 subunit proprotein, β-tubulin, s1c9a3r1, prosolin, HSP60, HER-2, L-plastin, estrogen receptor α, HSP27, thioredoxine peroxidase I, calumenin, 14-3-3 eta chain, Ki 67, MRP1, “similar to stratifin,” UCHL-1, mammaglobin 2, cellular RNA binding protein, p53, and annexin I;
b) a second set of probes for the detection of one or more marker genes selected from endogenous housekeeping genes; and
c) a detection means for identifying a probe hybridizing to a target marker gene.

79. The kit of claim 78, wherein the first set of probes is a plurality of nucleic acids complementary to mRNA encoding the selected marker genes.

80. The kit of claim 79, wherein the nucleic acid is selected from the group consisting of single-stranded RNA, double-stranded RNA, double-stranded DNA, single-stranded DNA, and RNA-DNA hybrids.

81. The kit of claim 78, wherein the second set of probes is a plurality of nucleic acids complementary to an mRNA encoding marker genes that do not vary statistically significantly in level of expression between cancer cell samples and normal cell samples.

82. The kit of claim 78, wherein the second set of probes is a plurality of nucleic acids complementary to an mRNA encoding EF-2 and EIF-4B.

83. The kit of claim 82, wherein the nucleic acid is selected from the group consisting of single-stranded RNA, double-stranded RNA, double-stranded DNA, single-stranded DNA, RNA-DNA hybrids, and siRNA.

84. The kit of claim 78, wherein the detection means is selected from the group consisting of fluorophores, chemical dyes, radiolabels, chemiluminescent compounds, colorimetric enzymatic reactions, chemiluminescent enzymatic reactions, magnetic compounds, and paramagnetic compounds.

85. The kit of claim 78, wherein the first set and second set of probes are attached to a solid support at predetermined positions.

86. The kit of claim 78, wherein the cancer being detected is selected from the group consisting of breast adenocarcinoma, infiltrating ductal carcinoma, ovarian carcinoma, serous adenocarcinoma, clear cell adenocarcinoma, endometrioid carcinoma, and mucinous adenocarcinoma.

87. The kit of claim 78, wherein a training is provided that comprises a pamphlet supplying information on the levels of expression of the marker genes in a normal cell sample and a neoplastic cell sample detected by the first set of probes.
